(19) United States
(12) Patent Application Publication
(10) Pub. No.: US 2023/0125329 A1
(43) Pub. Date: Apr. 27, 2023

(54) COMPOUND, ORGANIC ELECTROLUMINESCENCE MATERIAL, ORGANIC ELECTROLUMINESCENCE ELEMENT AND ELECTRONIC DEVICE

(71) Applicant: Ningbo Lumilan Advanced Materials Co., Ltd., Ningbo City (CN)

(72) Inventors: Xiangzhi LI, Ningbo City (CN); Ye CAI, Ningbo City (CN); Ting-Wei WEI, Ningbo City (CN); Zhi-Kuan CHEN, Ningbo City (CN)

(21) Appl. No.: 17/901,082

(22) Filed: Sep. 1, 2022

(30) Foreign Application Priority Data
Sep. 6, 2021 (CN) 202111038545.8

(51) Int. Cl. H01L 51/00; C07D 403/04; C07D 403/10; C07D 405/14; C07D 249/20; C07D 403/14

(52) U.S. Cl. CPC H01L 51/0067; C07D 403/04; H01L 51/0072; C07D 403/10; C07D 405/14; H01L 51/0073; C07D 249/20; C07D 403/14; H01L 51/0061; H01L 51/5012

(57) ABSTRACT

The present invention provides a compound, an organic electroluminescence material, an organic electroluminescence element and an electronic device. When the compound is used as the material of the organic functional layers, it makes the element have a lower driving voltage, a higher current efficiency and a longer service life.

### CROSS-REFERENCE TO RELATED APPLICATION

Pursuant to 35 U.S.C. § 119(a), this application claims the benefit of the priority to Chinese Patent Application No. 202111038545.8, filed on Sep. 6, 2021. The content of the prior application is incorporated herein by its entirety.

### BACKGROUND OF THE INVENTION

### 1. Field of the Invention

The present invention belongs to the field of organic electroluminescence material, which relates to a compound, an organic electroluminescence material, and an organic electroluminescence element and an electron electronic device comprising the same.

### 2. Description of the Prior Arts

In organic electroluminescence devices such as organic light-emitting diodes (OLEDs), which usually comprise an anode, a cathode and organic layers formed between the two electrodes, electricity is applied to convert electrical energy into light. An organic electroluminescence (EL) device may comprise organic layers such as a hole injection layer, a hole transport layer, a hole auxiliary layer, an emitting auxiliary layer, an electron blocking layer, an emitting layer (comprising a host material and a dopant material), an electron buffer layer, a hole blocking layer, an electron transport layer, an electron injection layer, and the like. The materials used in the organic layers can be categorized by their functions into a hole injection material, a hole transport material, a hole auxiliary material, an emitting auxiliary material, an electron blocking material, an emitting material, an electron buffer material, a hole blocking material, an electron transport material, an electron injection material, and the like. In an organic EL device, a voltage is applied to inject holes of the anode and electrons from the cathode into the emitting layer, and the recombination of the holes and electrons produces high energy excitons. The organic light-emitting compounds turn into the excited state by energy, and light is emitted when the organic light-emitting compounds at the excited state return to their base state.

Nowadays, the functional organic materials have properties such as insufficient stability and unbalanced charge carrier mobility, and these properties result in low light-emitting efficiency and short service life of organic electroluminescence devices, thereby greatly limiting the applications of organic electroluminescence devices.

Thus, in the field of the present invention, it is important to develop a novel high-performance functional organic material.

### SUMMARY OF THE INVENTION

To overcome the shortcomings of the existing technology, the objective of the present invention is to provide a compound, an organic electroluminescence material, and an organic electroluminescence element and an electronic device comprising the same.

To achieve the above objective, the present invention uses the following technical approaches:

In one aspect, the present invention provides a compound, and the compound has a structure represented by Formula (1):

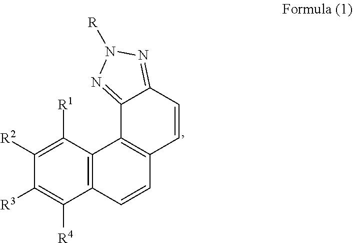

wherein, R is selected from hydrogen, deuterium, halogen, a cyano group, a substituted or unsubstituted C1-C30 alkyl group, a substituted or unsubstituted C3-C30 cycloalkyl group, a substituted or unsubstituted C6-C30 aryl group, and a substituted or unsubstituted C3-C30 heteroaryl group;

R¹ is -L¹Ar¹; R² is -L²Ar²; R³ is -L³Ar³; R⁴ is -L⁴Ar⁴;

L¹ to L⁴ are each independently selected from a bond, a substituted or unsubstituted C6-C30 arylene group, and a substituted or unsubstituted C3-C30 heteroarylene group; and

Ar¹ to Ar⁴ are each independently selected from hydrogen, deuterium, halogen, a cyano group, a substituted or unsubstituted C6-C60 arylamino group, a substituted or unsubstituted C3-C60 a heteroarylamino group, a substituted or unsubstituted C6-C60 aryl group, and a substituted or unsubstituted C3-C60 heteroaryl group.

In the present invention, the compound has an appropriate HOMO and LUMO energy level, which is aligned with the energy levels of adjacent layers, and this is advantageous for charge carrier transfer, so as to increase the emitting efficiency of the element. In addition, the compound of the present invention has a higher stability, and the element prepared by the compound has a longer service life.

Preferably, at least one of Ar¹ to Ar⁴ is selected from a group represented by Formula a:

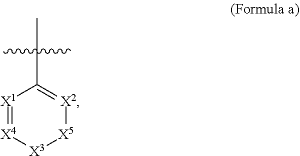

X¹ is selected from N and CR^{X1}; X² is selected from N and CR^{X2}; X³ is selected from N and CR^{X3}; X⁴ is selected from N and CR^{X4}; X⁵ is selected from N and CR^{X5};

R^{X1} to R^{X5} are each independently selected from hydrogen, deuterium, halogen, a cyano group, a substituted or unsubstituted C1-C30 alkyl group, a substituted or unsubstituted C3-C30 cycloalkyl group, a substituted or unsubstituted C6-C30 aryl group, a substituted or unsubstituted C3-C30 heteroaryl group; R^{X1} to R^{X5} are present individually without forming a ring, or any adjacent two of R^{X1} to R^{X5} joined to form a ring A, and the ring A is a substituted or unsubstituted C6-C30 aromatic ring, or a substituted or unsubstituted C3-C30 heteroaromatic ring.

Preferably, the ring A is a benzene ring, a naphthalene ring, an anthracene ring, a phenanthrene ring, a benzothiophene ring, a benzofuran ring, or an indene ring.

Preferably, X¹ is N; X² is N; X³ is CR^{X3}; X⁴ is CR^{X4}; and X⁵ is CR^{X5}.

Preferably, X¹ is N; X³ is N; X² is CR^{X2}; X⁴ is CR^{X4}; and X⁵ is CR^{X5}.

Preferably, X¹ is N; X² is N; X³ is N; X⁴ is CR^{X4}; and X⁵ is CR^{X5}.

Preferably, the Formula a is selected from

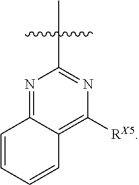

Preferably, the Formula a is selected from

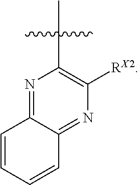

Preferably, R^{X1} to R^{X5} are each independently selected from hydrogen, deuterium, halogen, and a group selected from a phenyl group, a biphenylyl group, a terphenylyl group, a naphthyl group, a phenanthryl group, an anthryl group, a phenylnaphthyl group, a naphthylphenyl group, a pyridyl group, a bipyridyl group, a dibenzofuryl group, a dibenzothiophenyl group, a carbazolyl group, a carbazolylphenyl group, a phenylcarbazolyl group, a dimethylfluorenyl group, a diphenylfluorenyl group, a spiro-bifluorenyl group, a dibenzofurylphenyl group, a dibenzothiophenylphenyl group, a dimethylfluorenylphenyl group, a benzocarbazolyl group, a benzonaphthofuryl group and a benzonaphthothiophenyl group, each of which is substituted or unsubstituted.

Preferably, Ar¹ to Ar⁴ are each independently selected from hydrogen, deuterium, halogen, and a group selected from a phenyl group, a naphthyl group, a biphenylyl group, a phenanthryl group, a fluoranthenyl group, a benzophenanthryl group, a terphenylyl group, a triphenylenylene group, a dimethylfluorenyl group, a diphenylfluorenyl group, a spiro-bifluorenyl group, a benzodimethylfluorenyl group, a benzodiphenylfluorenyl group, a benzo-spiro-bifluorenyl group, a dibenzofuryl group, a benzonaphthofuryl group, a benzonaphthothiophenyl group, and a dibenzothiophenyl group, each of which is substituted or unsubstituted.

Preferably, at least one of Ar¹ to Ar⁴ is selected from a group represented by Formula b:

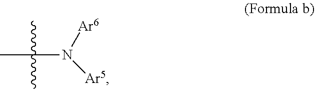

Ar⁵ to Ar⁶ are each independently selected from a substituted or unsubstituted C6-C30 aryl group, and a substituted or unsubstituted C3-C30 heteroaryl group.

Preferably, Ar⁵ to Ar⁶ are each independently selected from a phenyl group, a biphenylyl group, a terphenylyl group, a naphthyl group, a phenanthryl group, an anthryl group, a triphenylenylene group, a phenylnaphthyl group, a naphthylphenyl group, a pyridyl group, a bipyridyl group, a dibenzofuryl group, a dibenzothiophenyl group, a benzonaphthofuryl group, a benzonaphthothiophenyl group, a dinaphthofuryl group, a dinaphthothiophenyl group, a dibenzofurylphenyl group, a dibenzothiophenylphenyl group, a dimethylfluorenyl group, a benzodimethylfluorenyl group, a diphenylfluorenyl group, a spiro-bifluorenyl group, and a dimethylfluorenylphenyl group, each of which is substituted or unsubstituted.

Preferably, at least one of Ar¹ to Ar⁴ is selected from a group represented by Formula c:

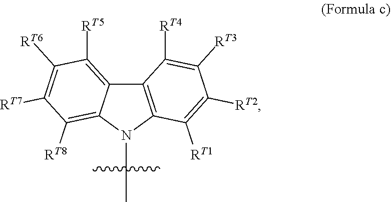

R^{T1} to R^{T8} are each independently selected from hydrogen, deuterium, halogen, a cyano group, a substituted or unsubstituted C1-C30 alkyl group, a C1-C30 alkyl group in which one or more methylene groups are independently substituted by —O— and/or —S— in a manner that O atom and/or S atom are not adjacent to each other, a substituted or unsubstituted C7-C30 arylalkyl group, a substituted or unsubstituted C6-C30 aryl group, a substituted or unsubstituted C3-C30 heteroaryl group, a substituted or unsubstituted C4-C30 heteroarylalkyl group, a substituted or unsubstituted C3-C30 cycloalkyl group, a substituted or unsubstituted C3-C30 heterocycloalkyl group, a substituted or unsubstituted C3-C30 cycloalkenyl group, a substituted or unsubstituted C1-C30 alkoxy group, and a substituted or unsubstituted C6-C30 aryloxy group;

R^{T1} to R^{T8} are present individually without forming a ring, or any adjacent two of R^{T1} to R^{T8} joined to form a ring B, and the ring B is a substituted or unsubstituted C6-C30 aromatic ring, or a substituted or unsubstituted C6-C30 heteroaromatic ring.

Preferably, the ring B is a benzene ring or a naphthalene ring.

Preferably, the Formula c is selected from any of the c-1, c-2, c-3, c-4, c-5 and c-6:

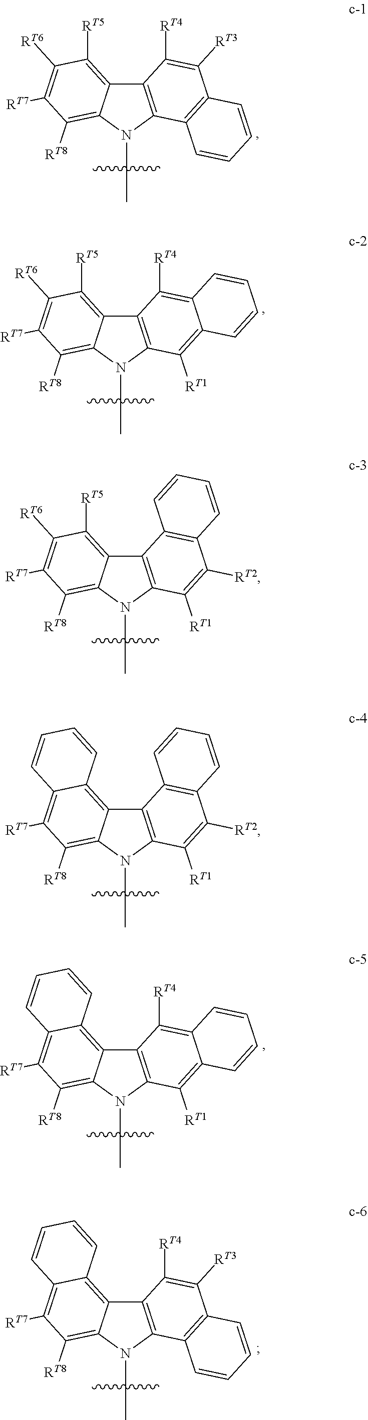

Preferably, R^{T1} to R^{T8} are each independently selected from hydrogen, deuterium, and a group selected from a methyl group, an ethyl group, a tert-butyl group, an adamantyl group, a phenyl group, a biphenylyl group, a terphenylyl group, a naphthyl group, a phenanthryl group, an anthryl group, a triphenylenylene group, a phenylnaphthyl group, a naphthylphenyl group, a pyridyl group, a bipyridyl group, a dibenzofuryl group, a dibenzothiophenyl group, a benzonaphthofuryl group, a benzonaphthothiophenyl group, a dinaphthofuryl group, a dinaphthothiophenyl group, a dibenzofurylphenyl group, a dibenzothiophenylphenyl group, a dimethylfluorenyl group, a benzodimethylfluorenyl group, a diphenylfluorenyl group, a spiro-bifluorenyl group and a dimethylfluorenylphenyl group, each of which is substituted or unsubstituted.

Preferably, L¹ to L⁴ are each independently selected from a bond, a substituted or unsubstituted phenylene group, a substituted or unsubstituted biphenylene group, a substituted or unsubstituted terphenylene group, and a substituted or unsubstituted naphthylene group.

Preferably, the R is selected from a phenyl group and a biphenylyl group, each of which is substituted or unsubstituted.

Preferably, when the above-mentioned group has one or more substituents, the substituents are each independently selected from deuterium, halogen, a cyano group, a nitro group, an unsubstituted or R′-substituted C1-C4 straight or branched alkyl group, an unsubstituted or R′-substituted C6-C20 aryl group, an unsubstituted or R′-substituted C3-C20 heteroaryl group, and an unsubstituted or R′-substituted C6-C20 arylamino group; R′ is selected from deuterium, halogen, a cyano group and a nitro group.

Preferably, the aryl group is selected from a phenyl group, a biphenylyl group, a terphenylyl group, a naphthyl group, an anthryl group, a phenanthryl group, a benzophenanthryl group, a naphthylphenyl group, a dimethylfluorenyl group, a diphenylfluorenyl group or a spiro-bifluorenyl group.

Preferably, the heteroaryl group is selected from a pyridyl group, a dibenzofuryl group, a dibenzothiophenyl group, a carbazolyl group, a phenylcarbazolyl group, a pyridylcarbazolyl group, a naphthylcarbazolyl group, a biphenylylcarbazolyl group, a dibenzofurylphenyl group, a dibenzothiophenylphenyl group, a benzonaphthofuryl group, a benzonaphthothiophenyl group, a benzocarbazolyl group and a dibenzocarbazolyl group.

Preferably, the alkyl group is selected from a methyl group, an ethyl group, a propyl group, a tert-butyl group, a cyclohexyl group and adamantyl.

Preferably, at least one of the R¹, R², R³ and R⁴ is selected from hydrogen.

Preferably, at least two of the R¹, R², R³ and R⁴ are selected from hydrogen.

Preferably, at least three of the R¹, R², R³ and R⁴ are selected from hydrogen.

Preferably, the R² is selected from -L²Ar²; and R¹, R³ and R⁴ are all selected from hydrogen.

Preferably, the R³ is selected from -L³Ar³; and R¹, R² and R⁴ are all selected from hydrogen.

Preferably, the compound is a compound M having electron transport properties.

Preferably, the compound is any one of compounds M1 to M206:

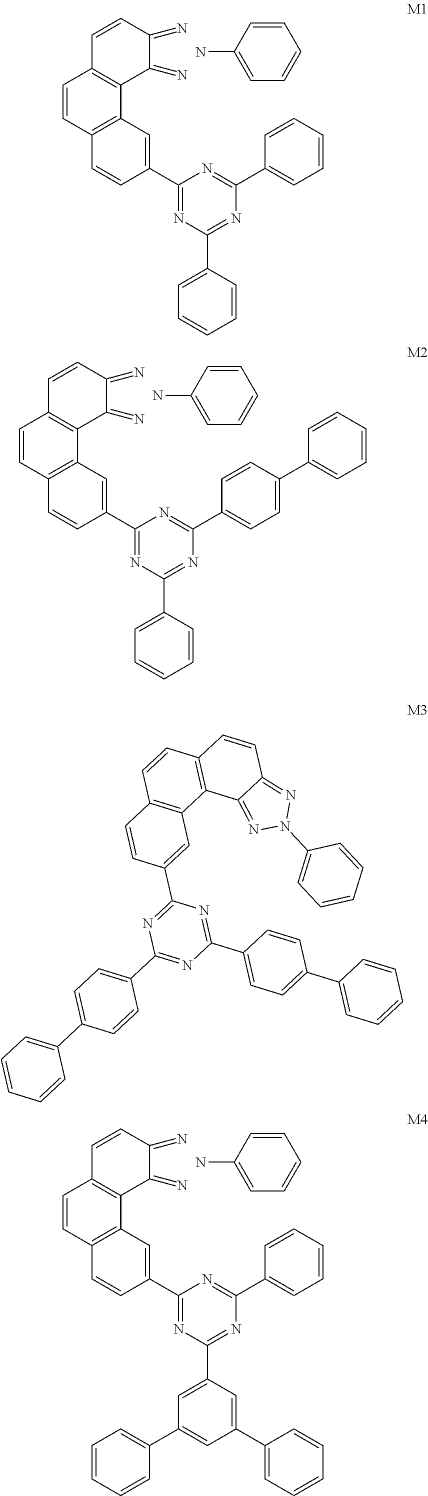

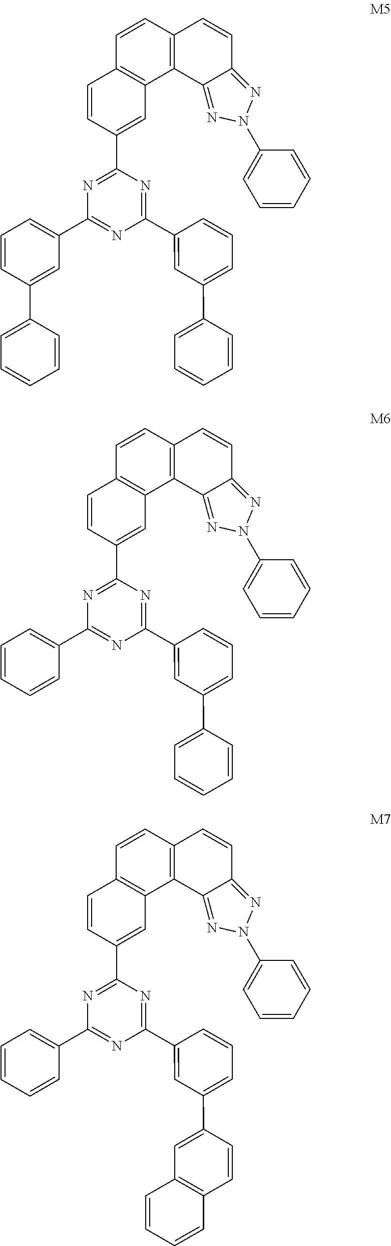

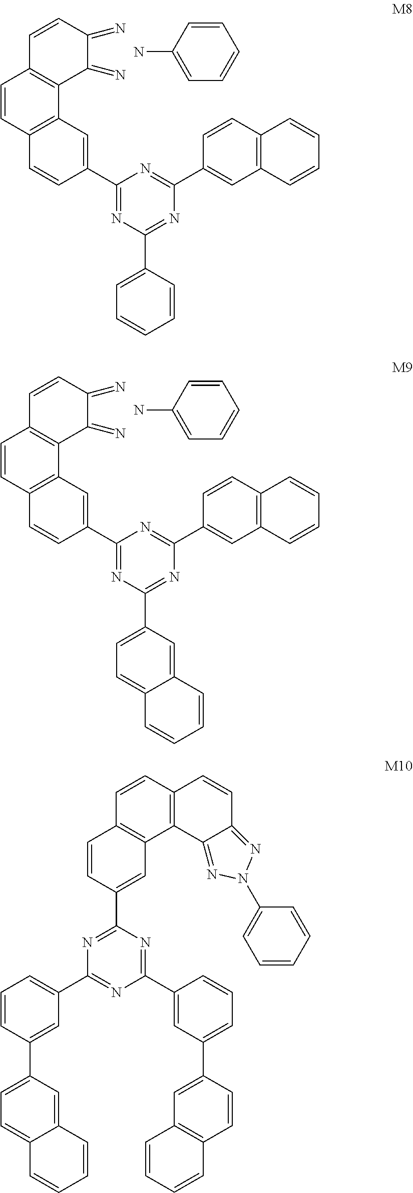

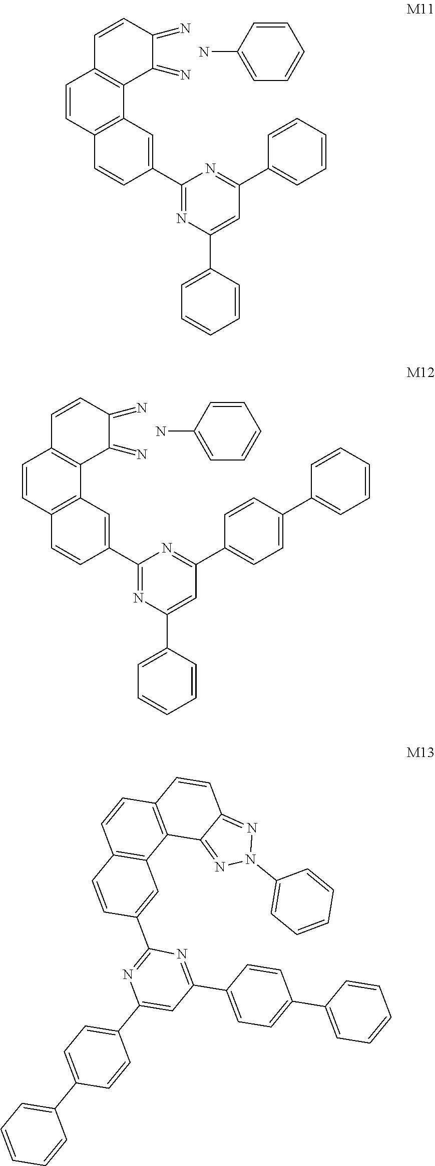

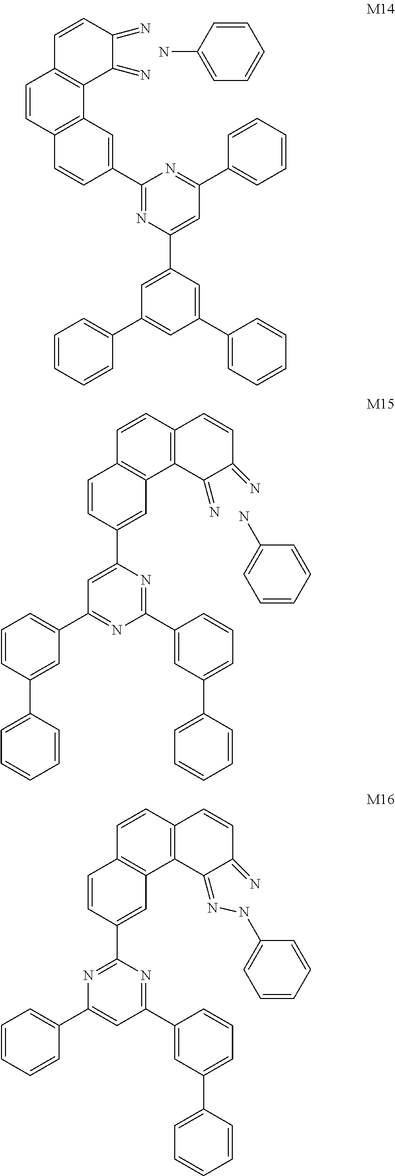

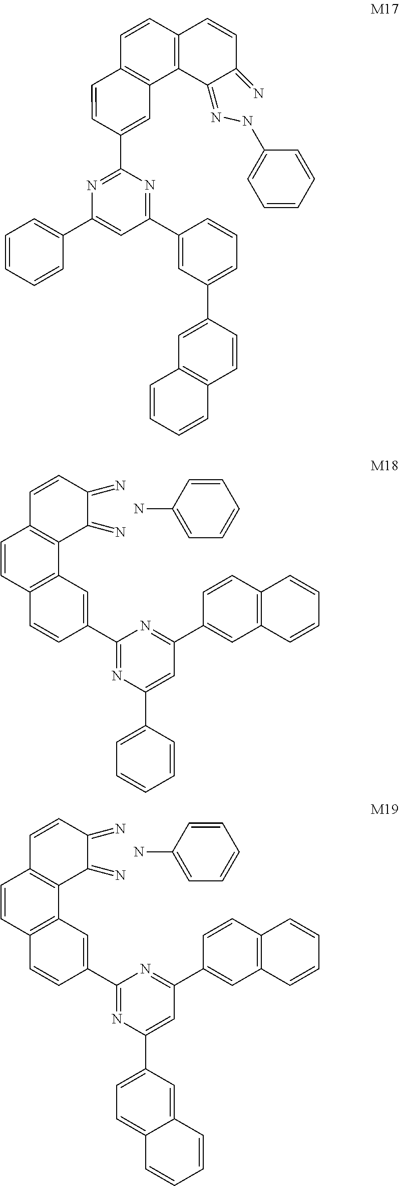

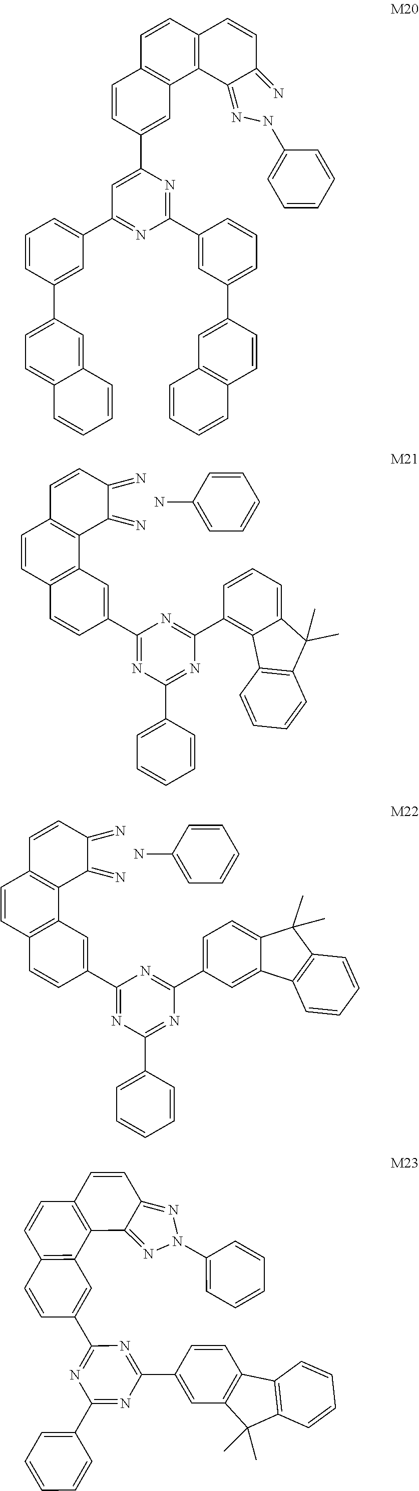

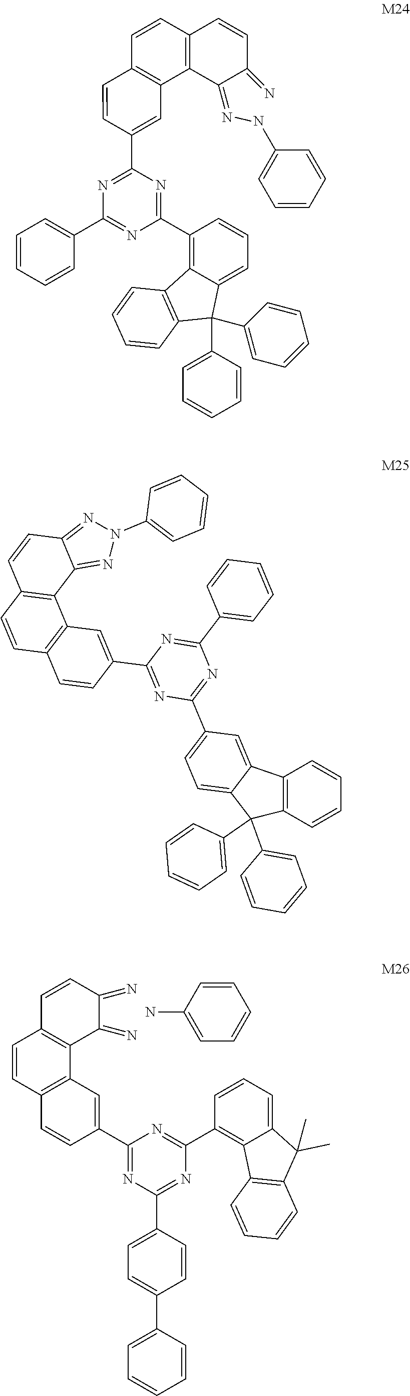

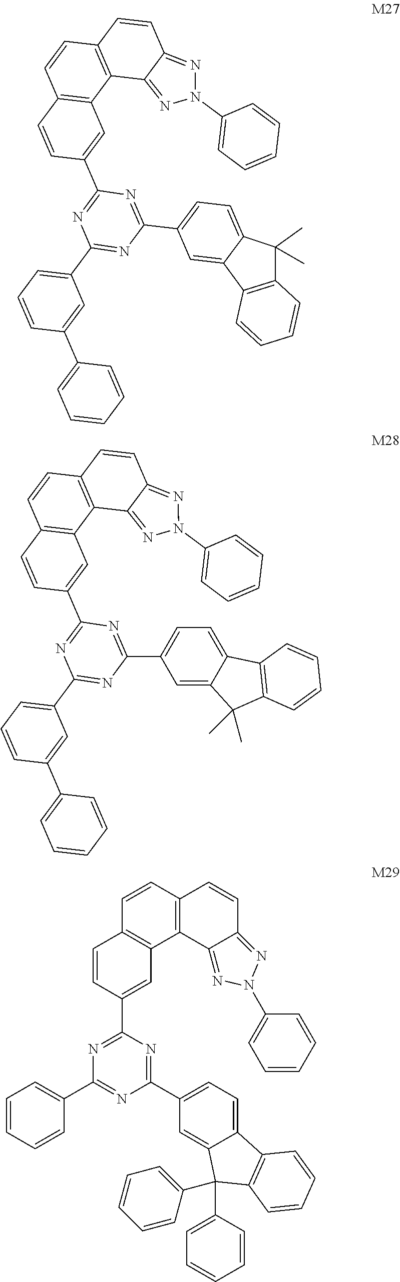

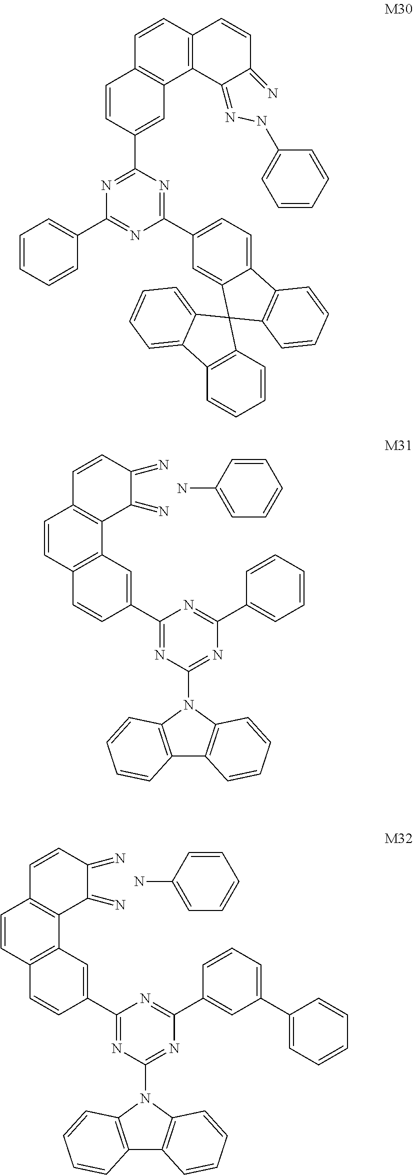

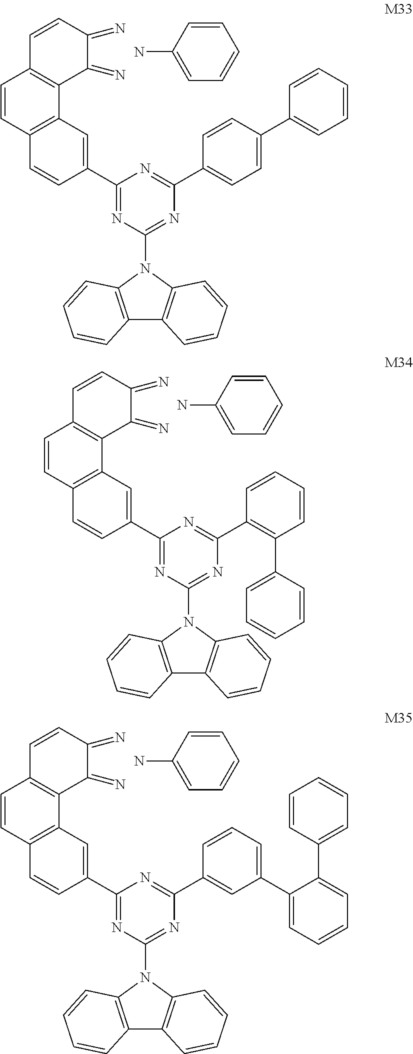

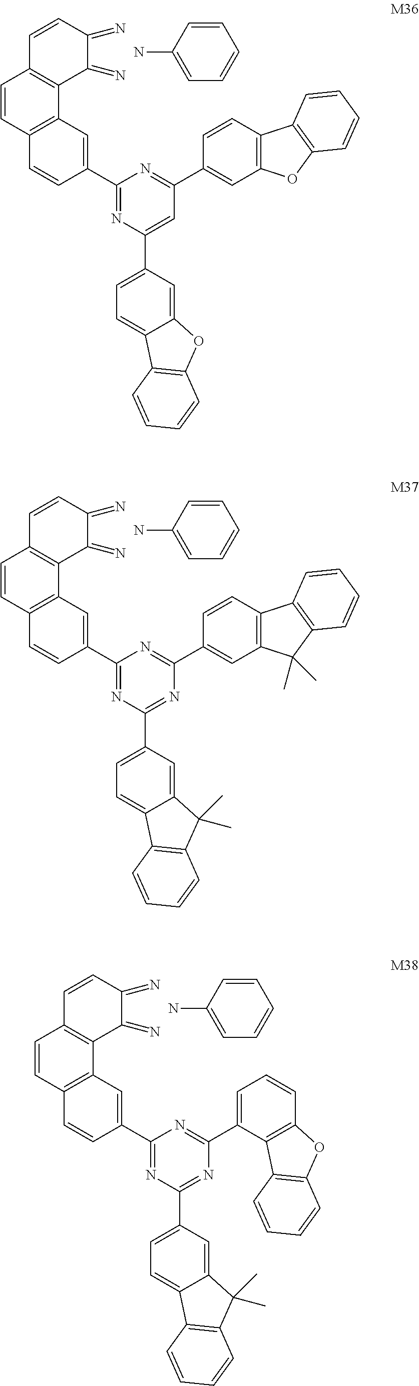

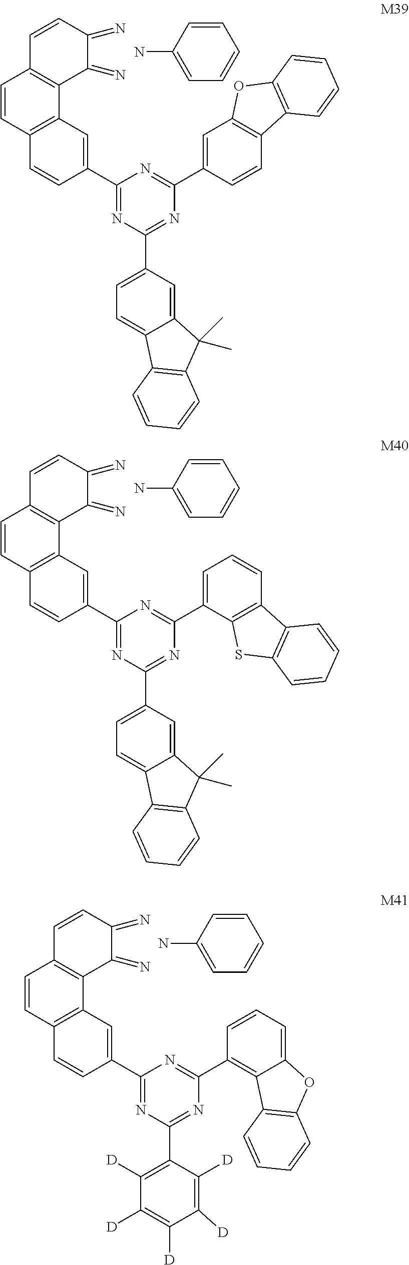

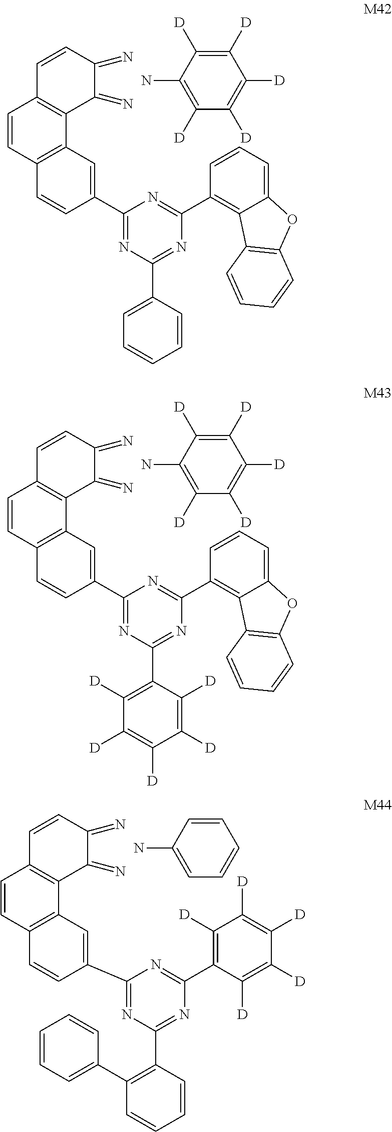

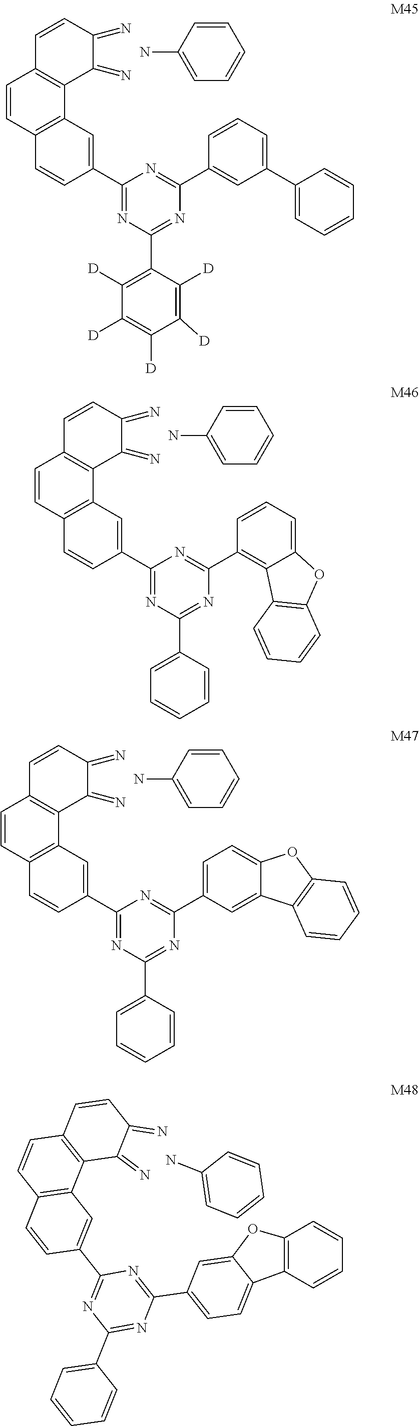

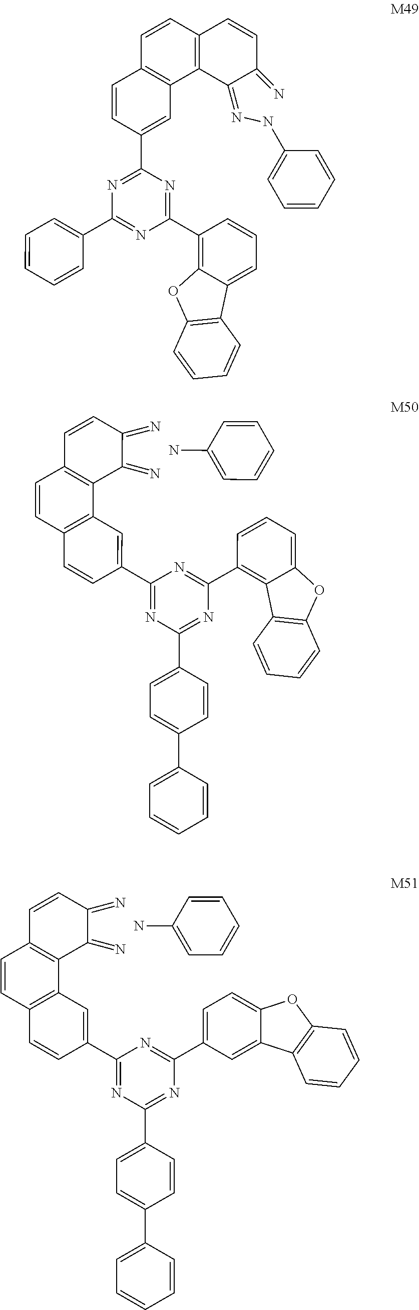

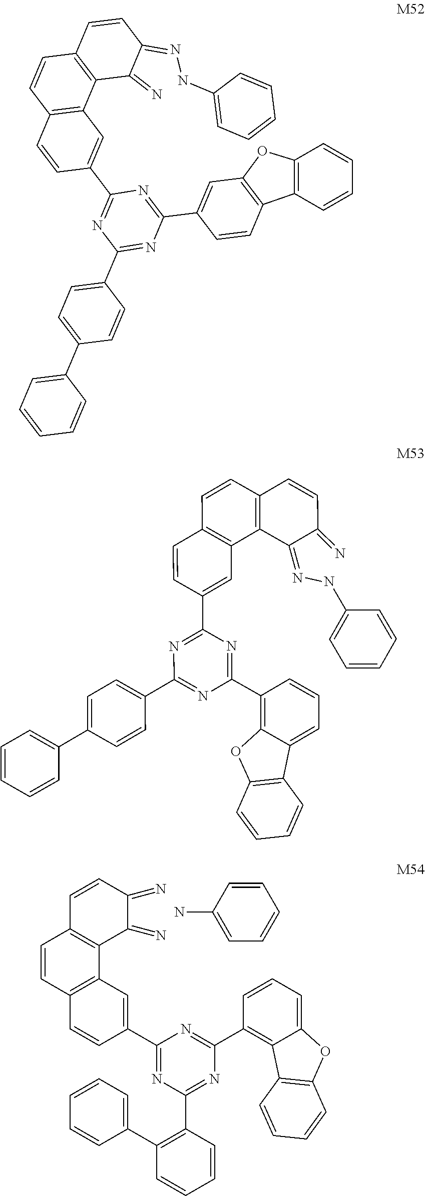

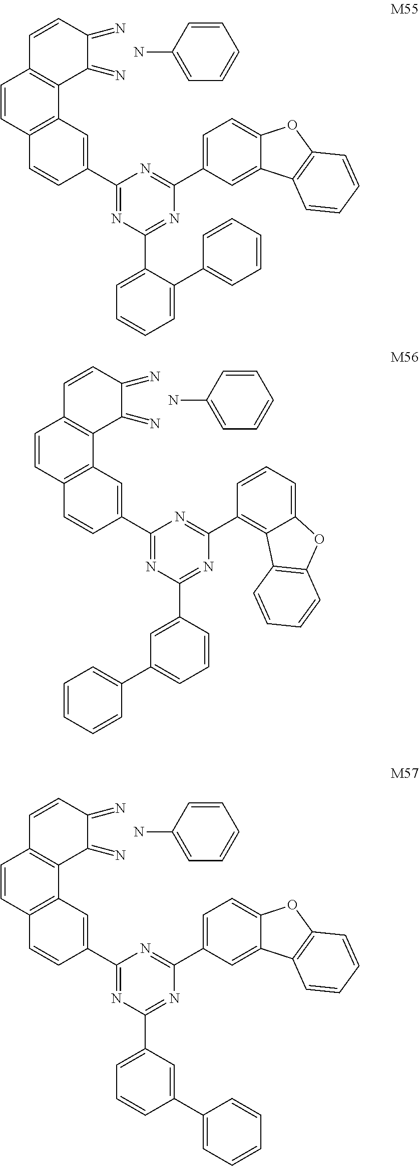

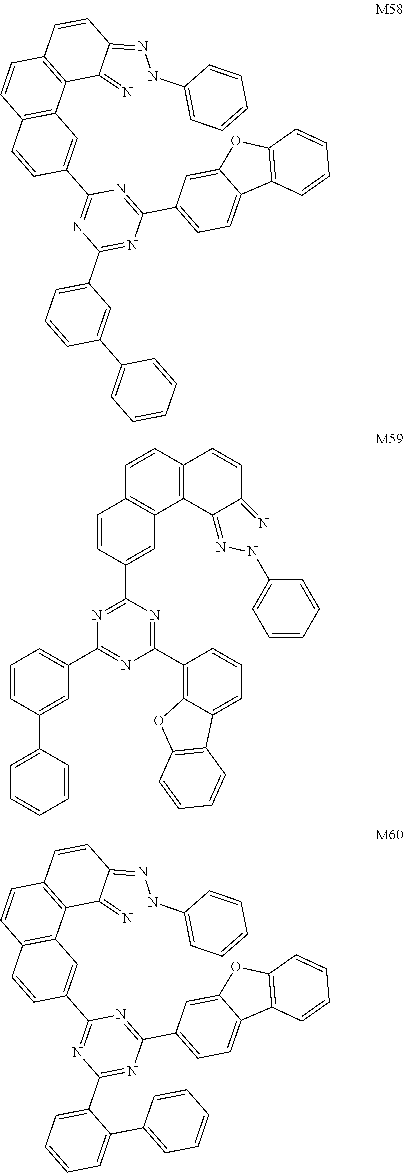

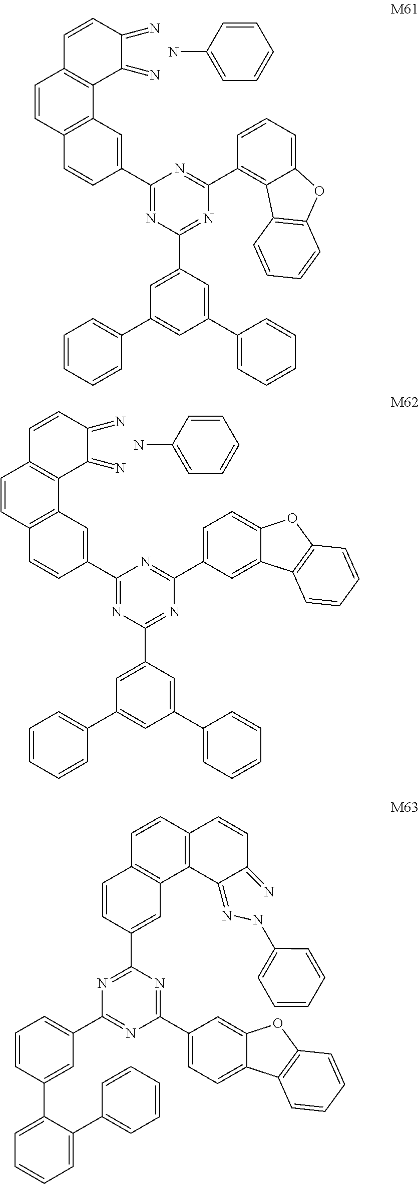

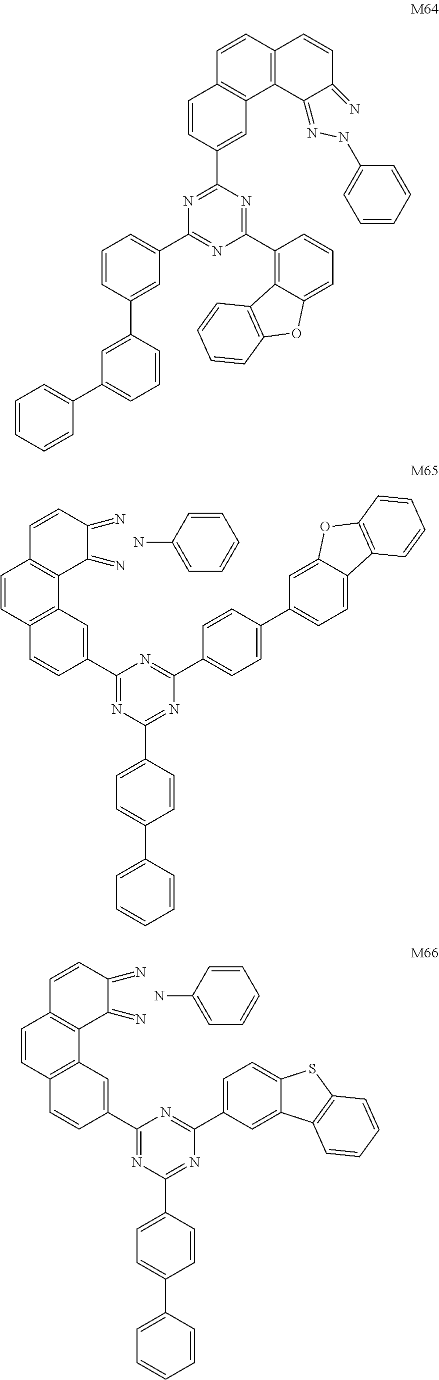

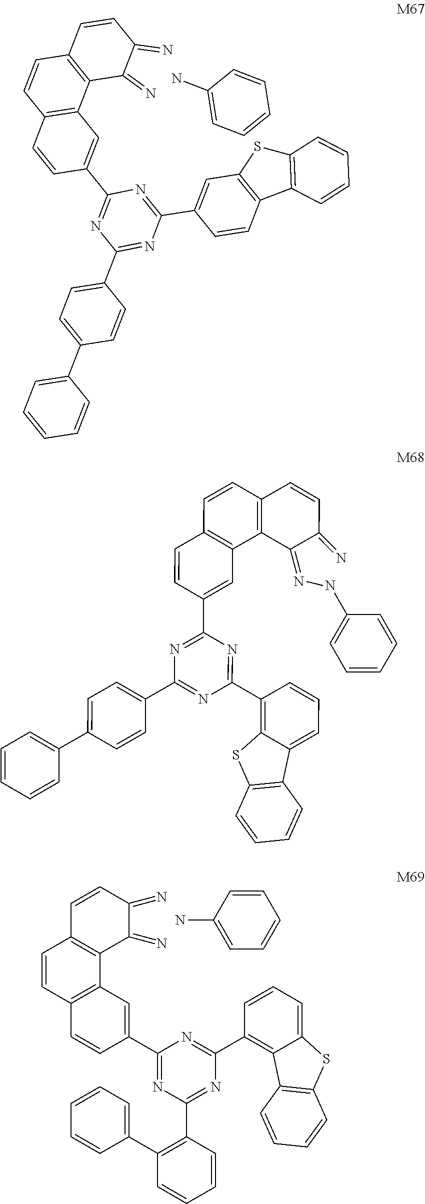

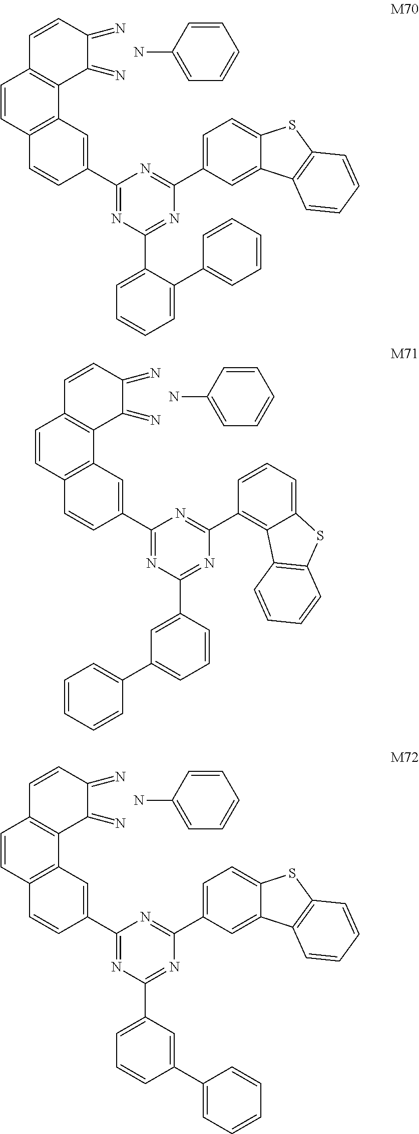

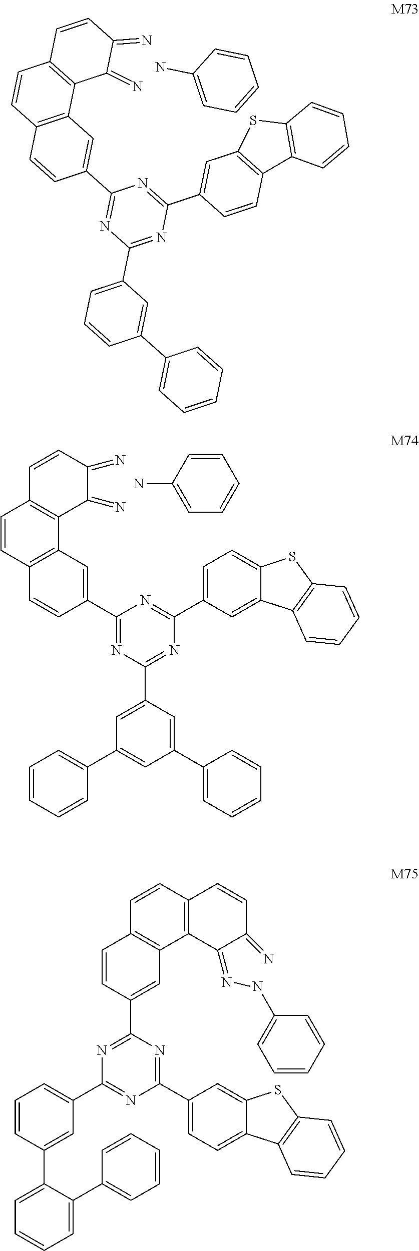

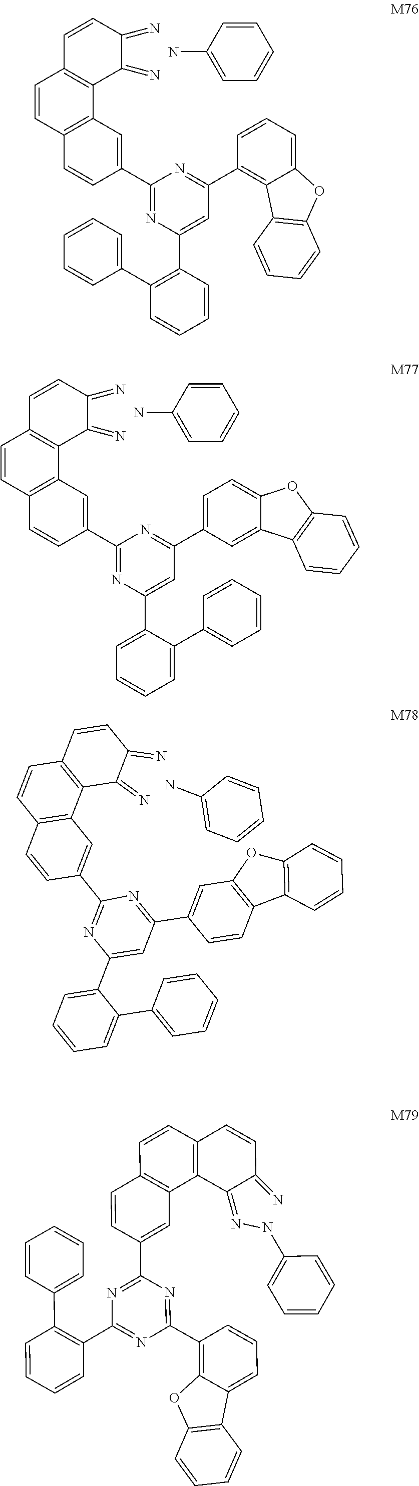

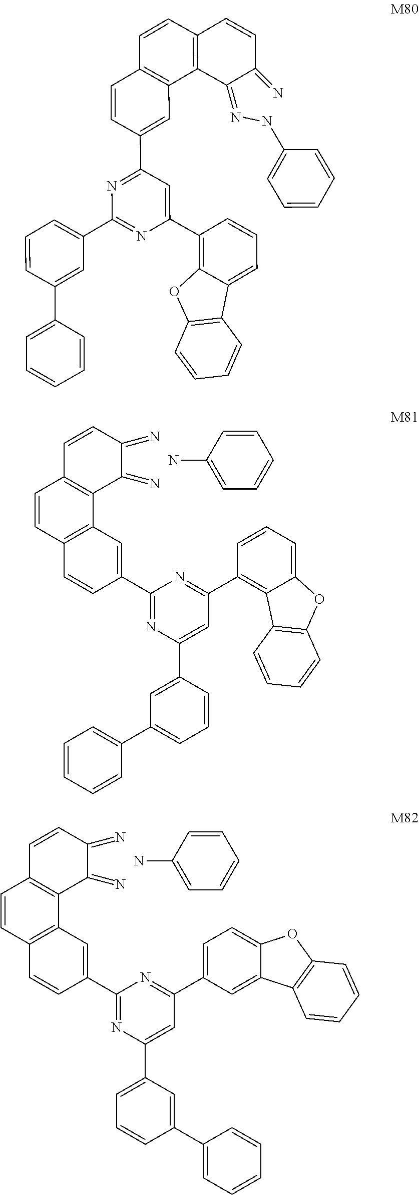

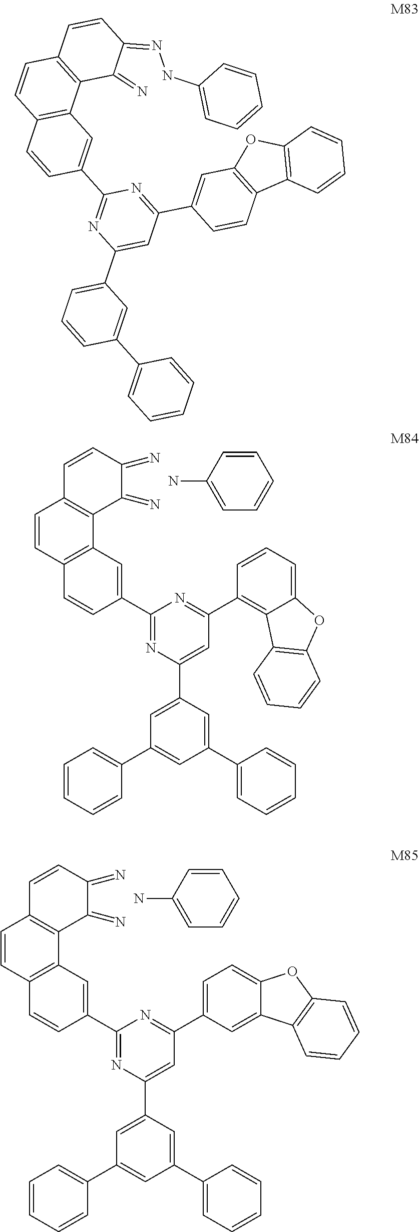

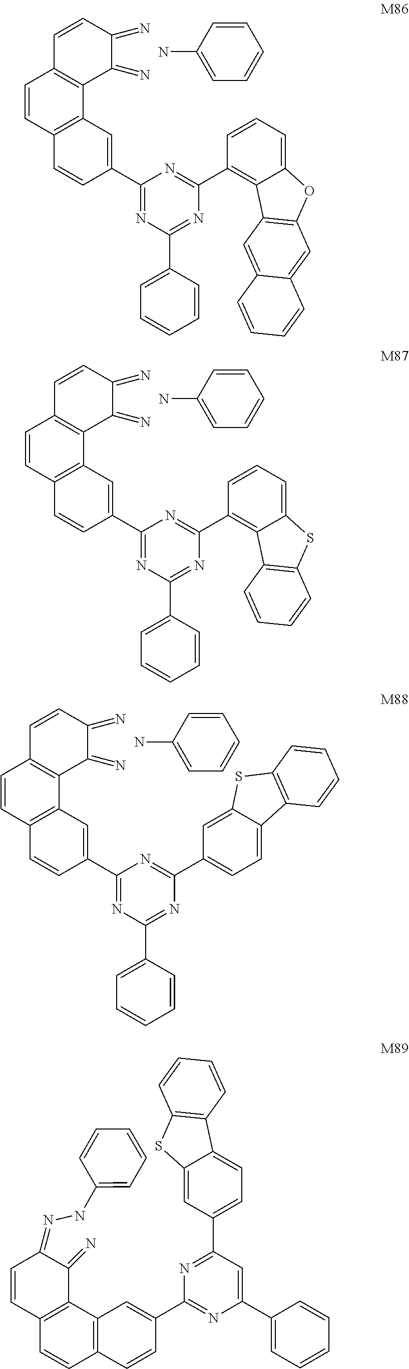

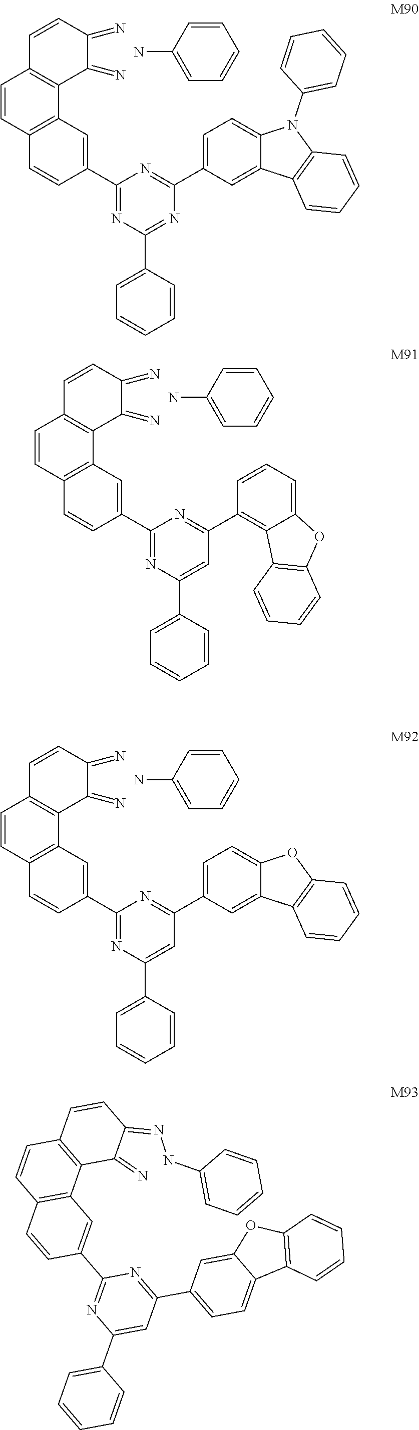

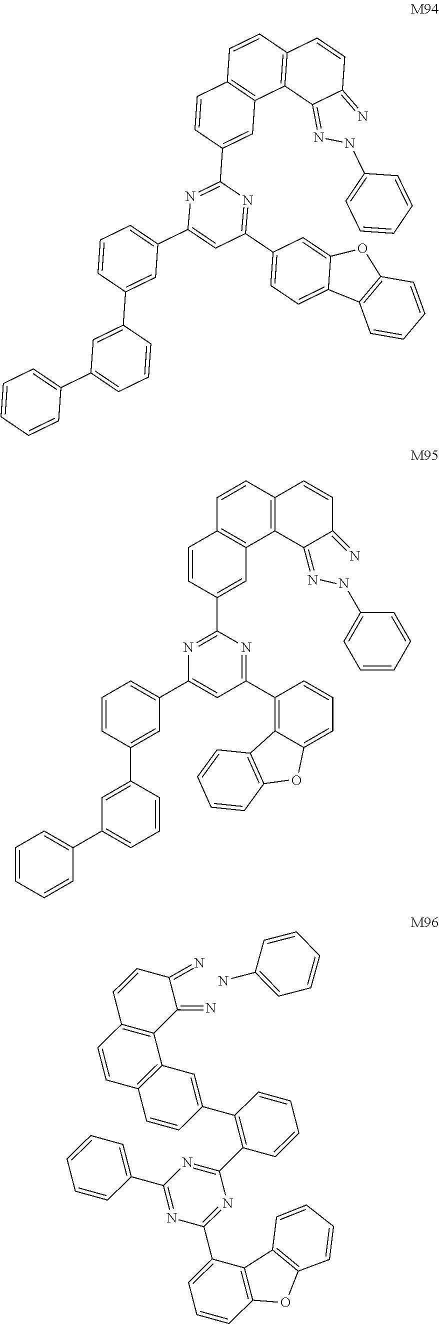

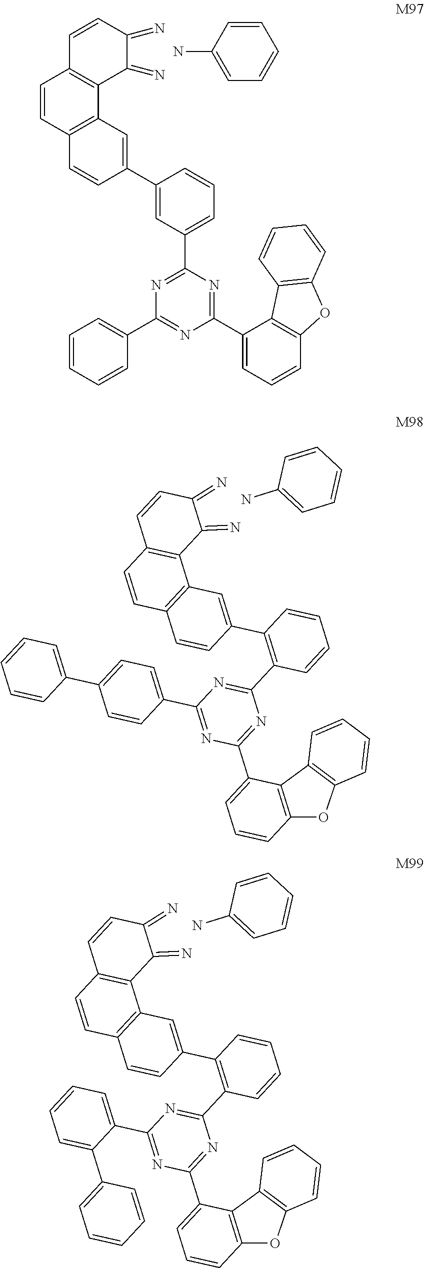

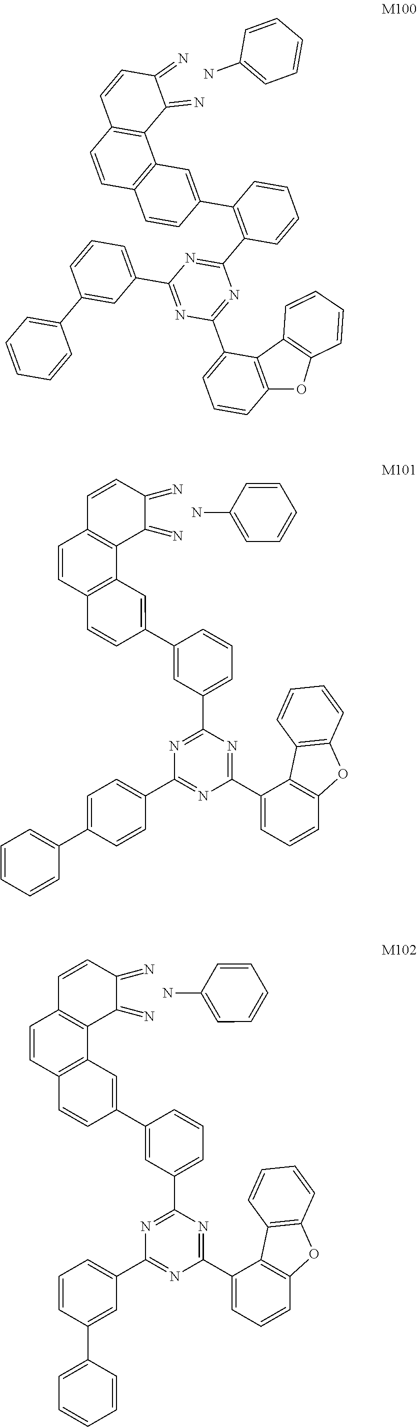

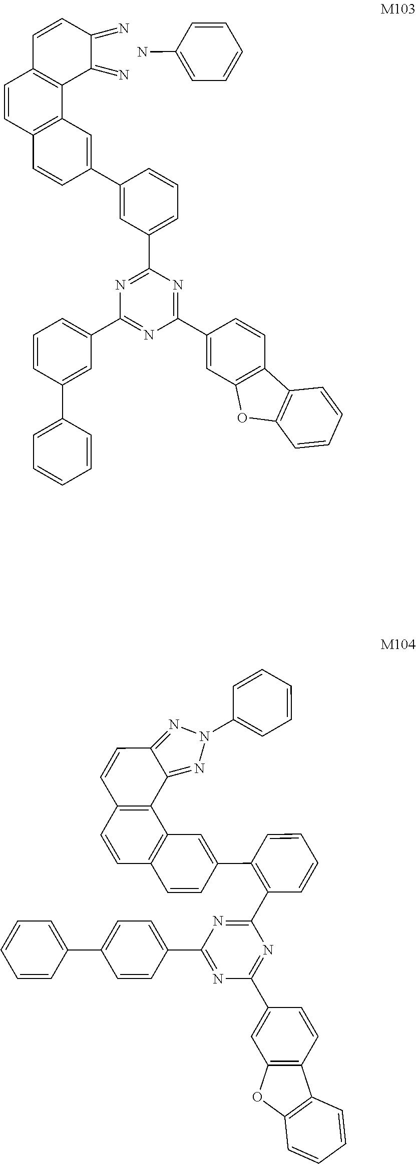

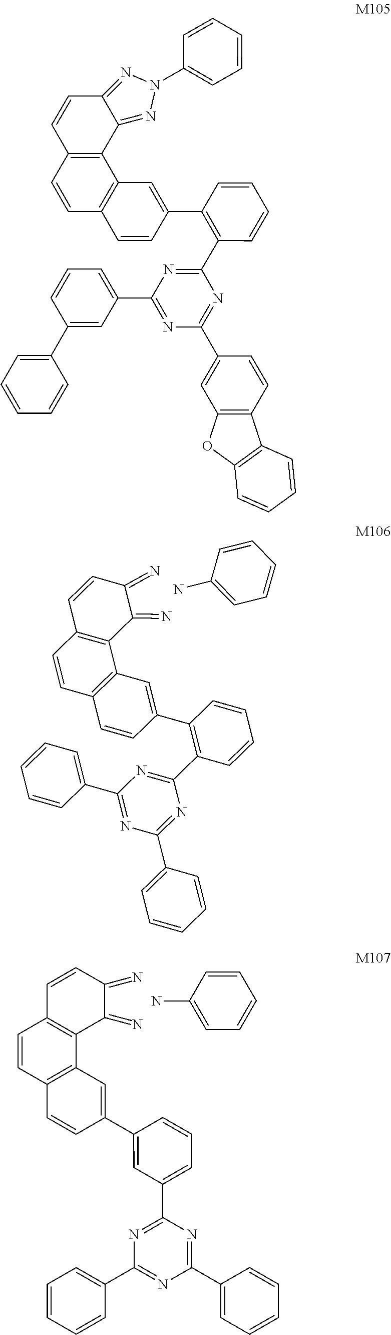

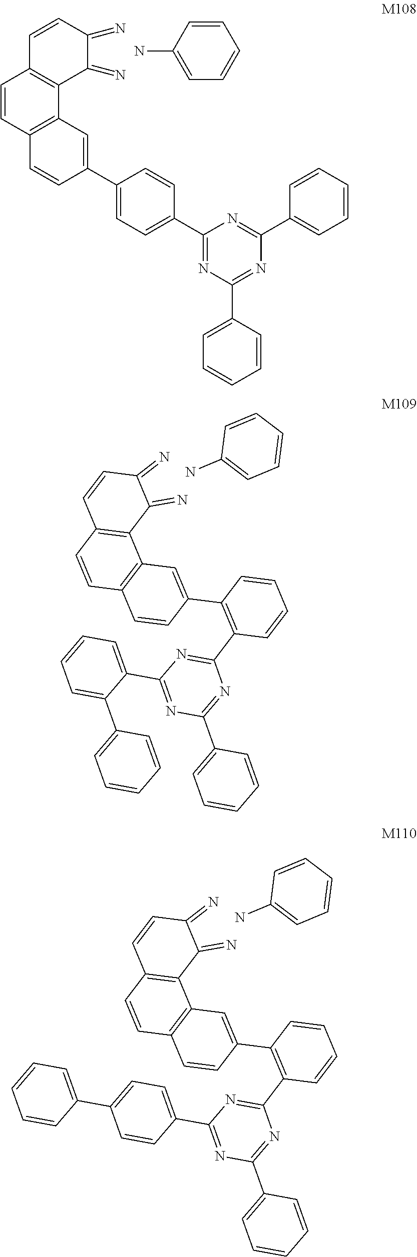

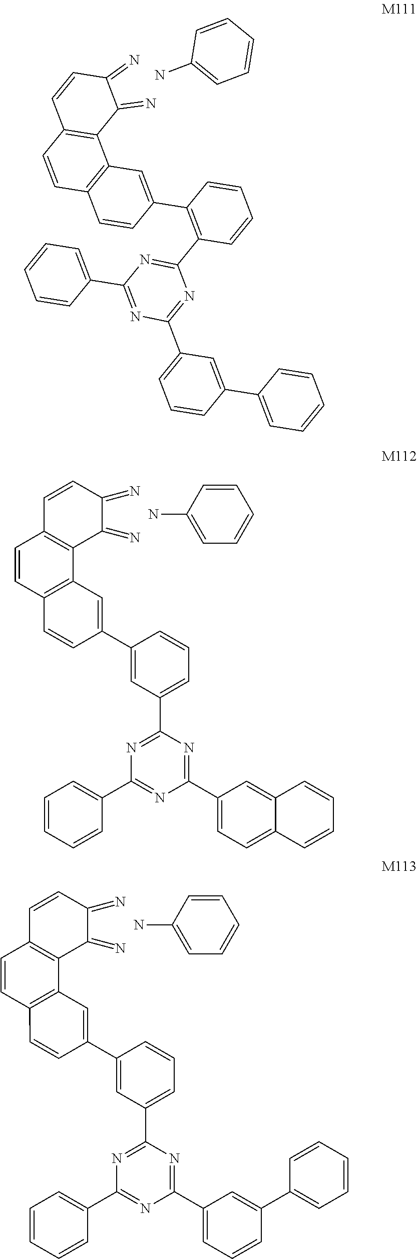

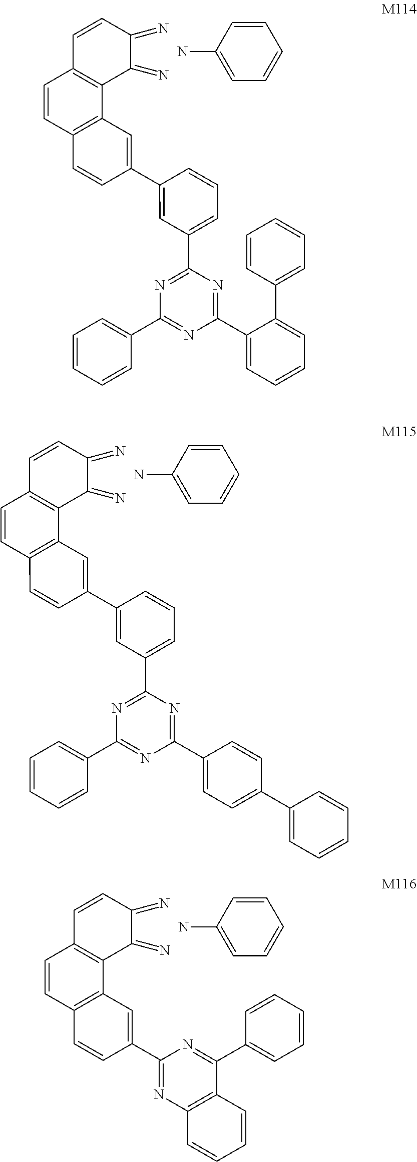

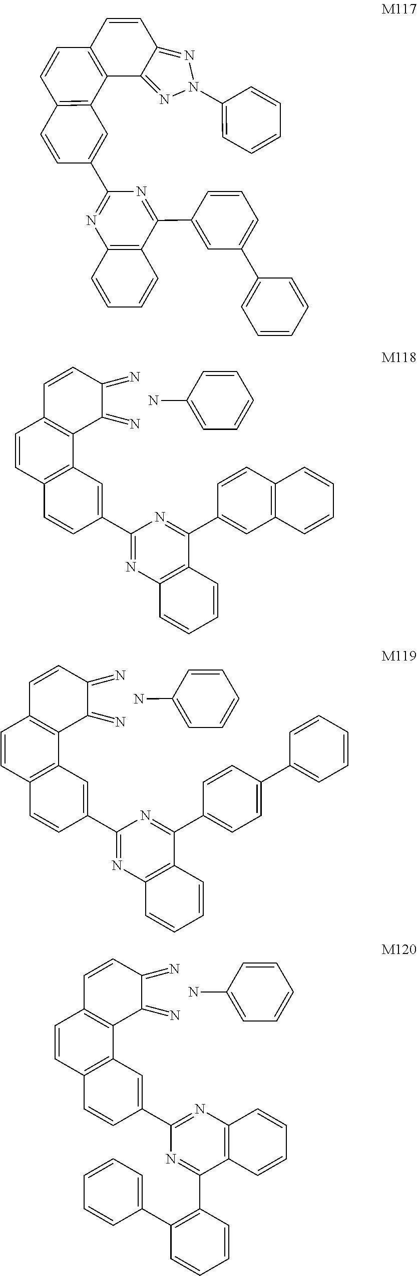

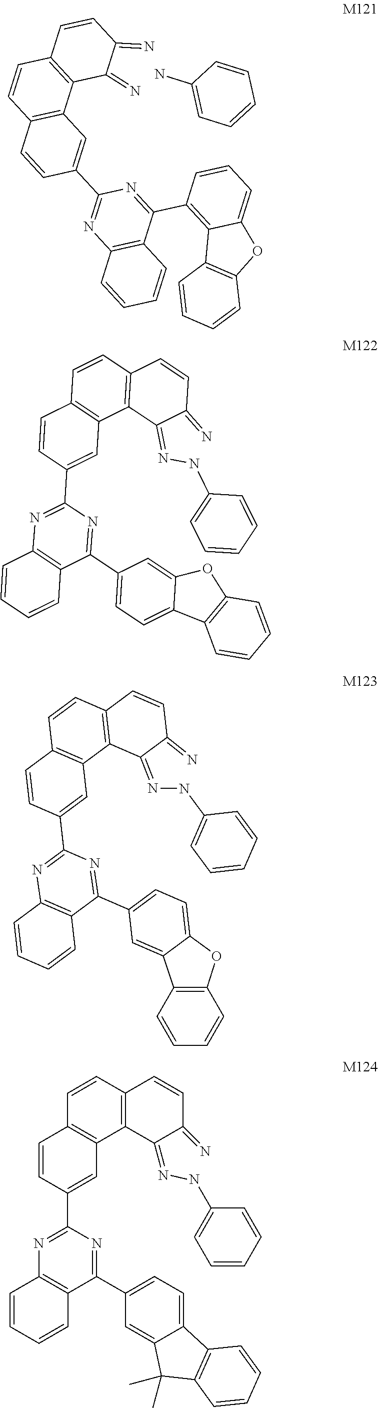

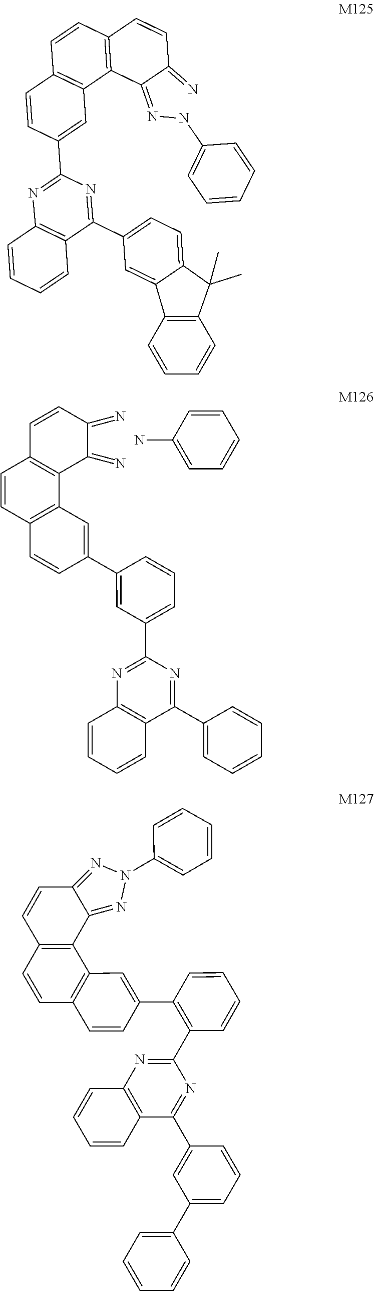

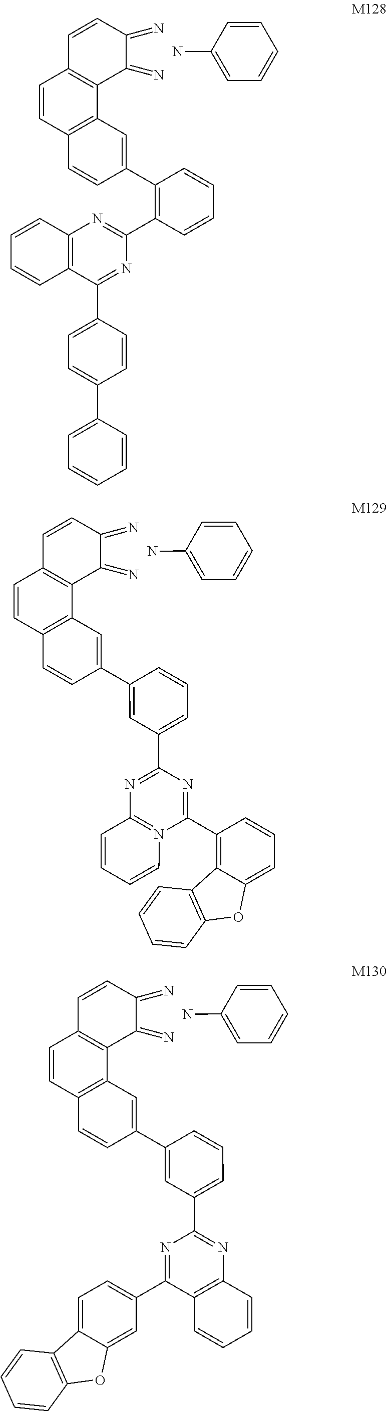

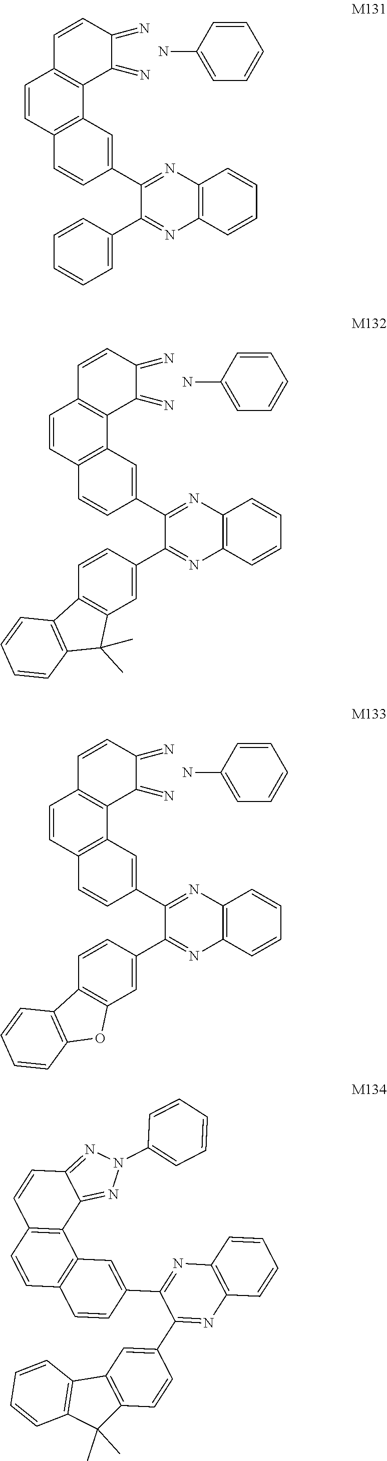

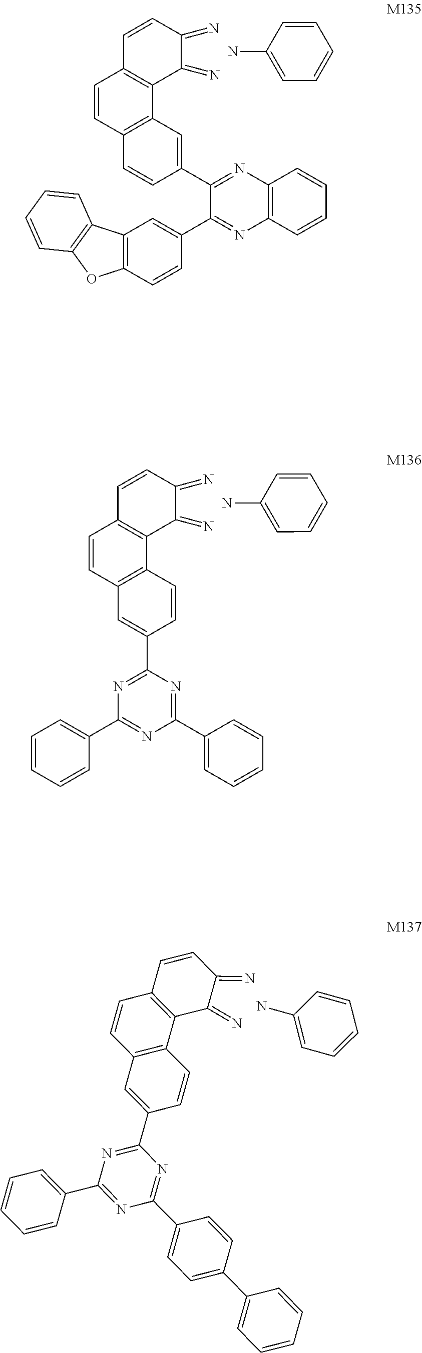

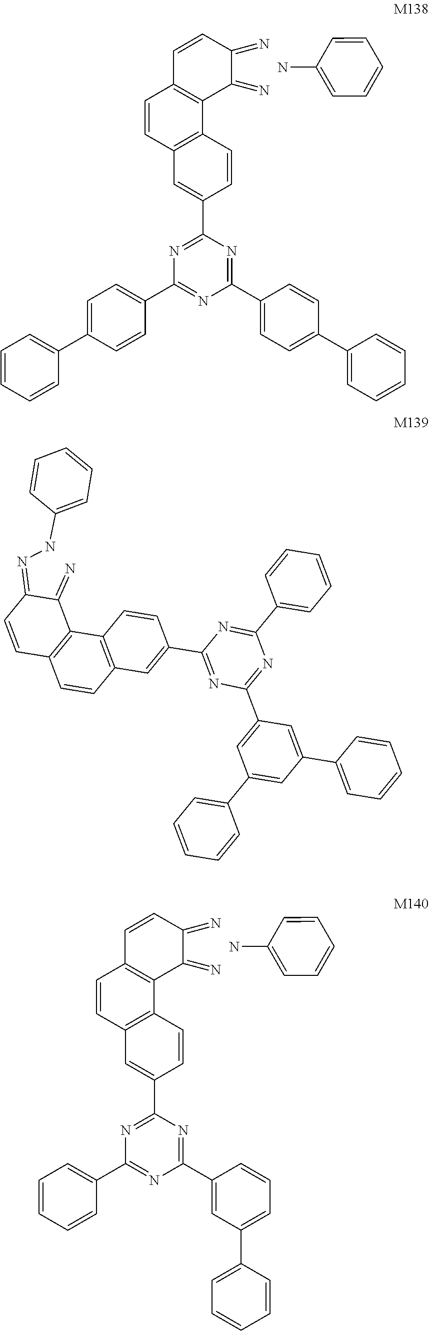

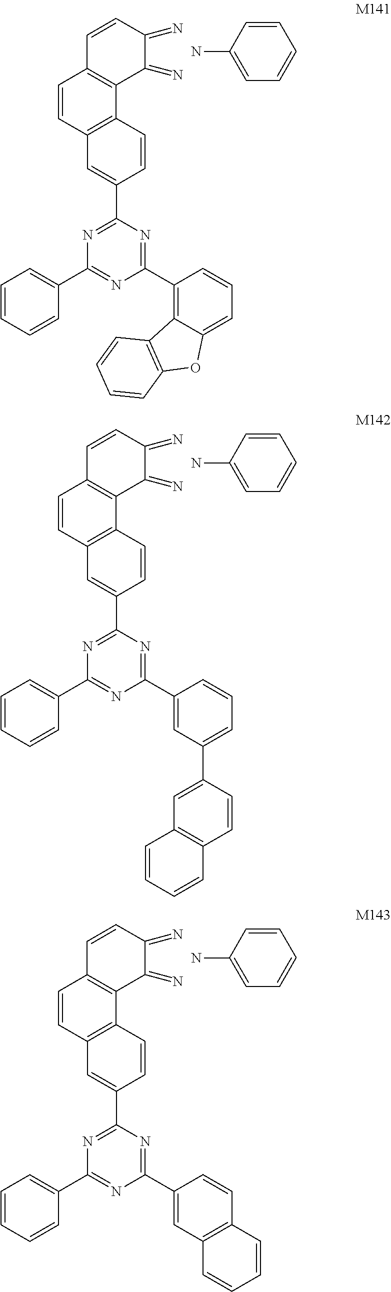

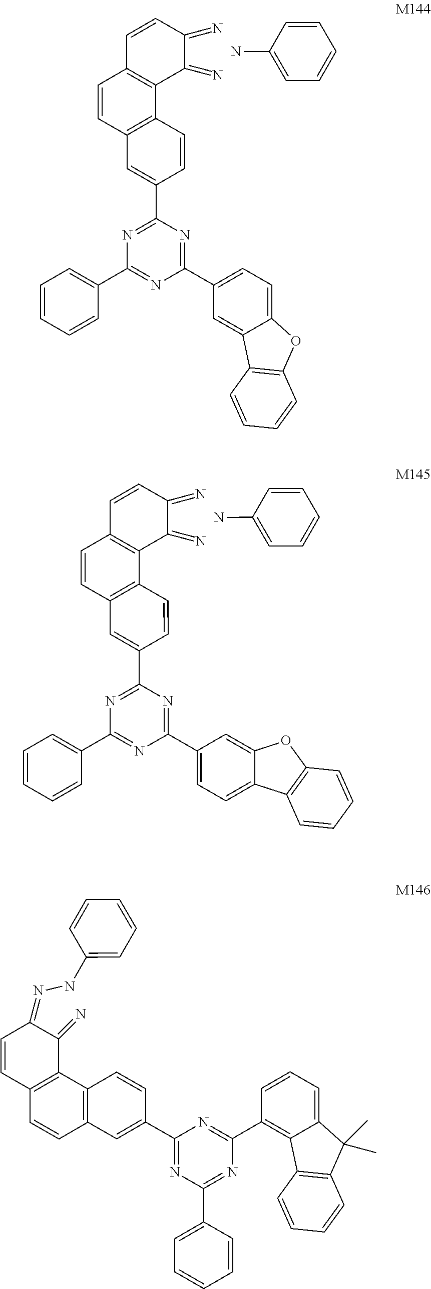

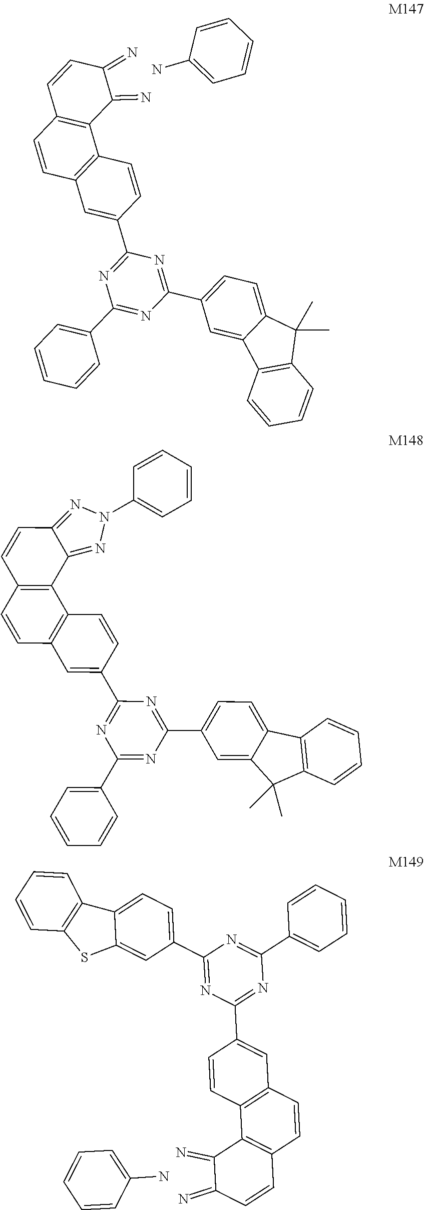

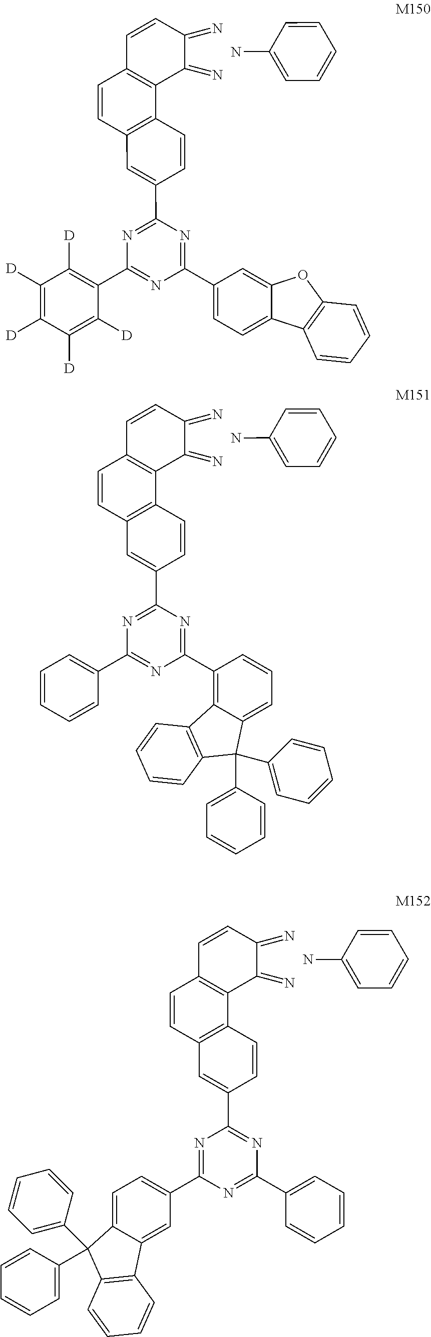

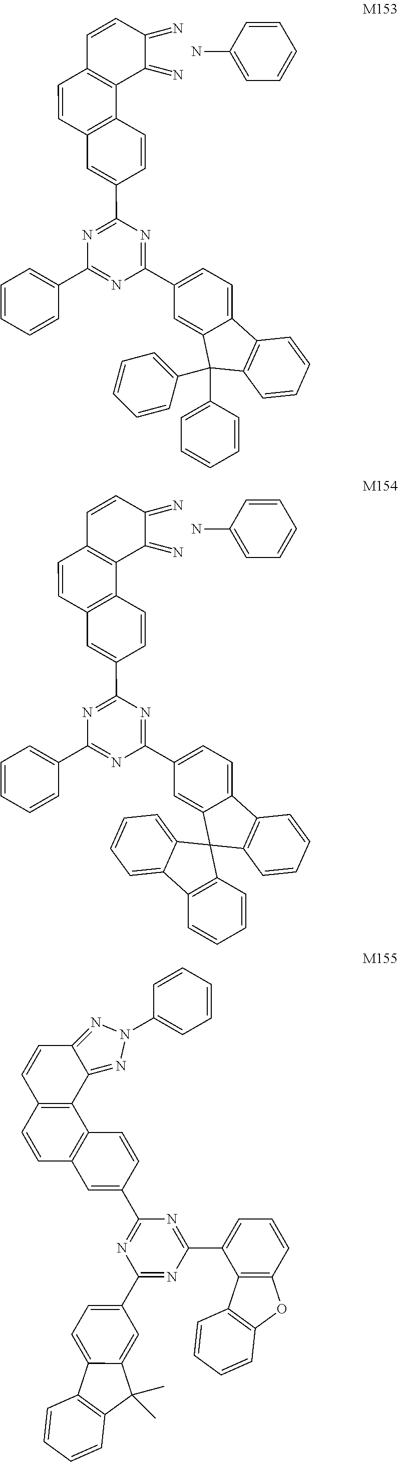

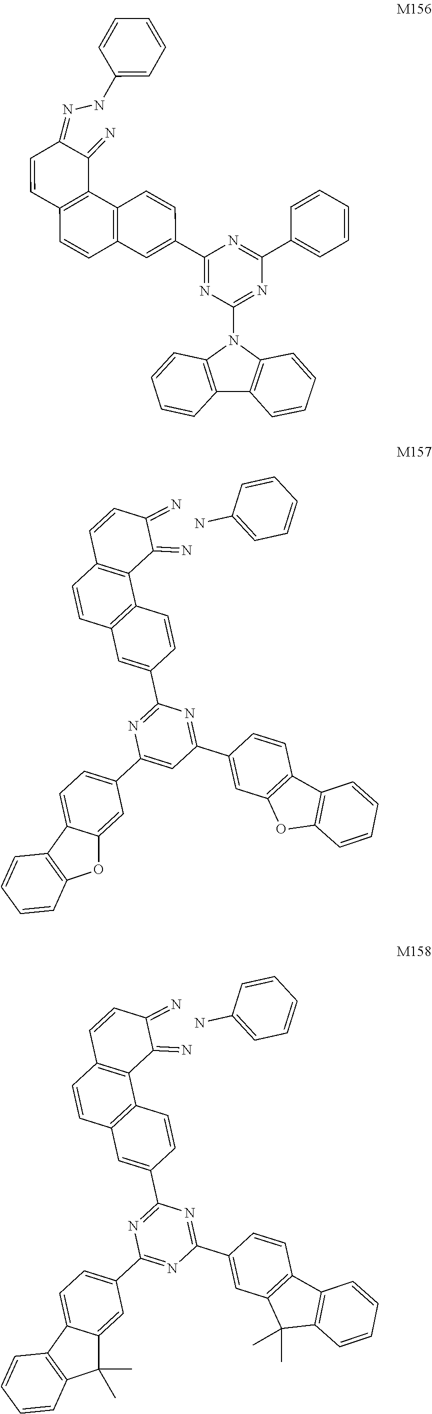

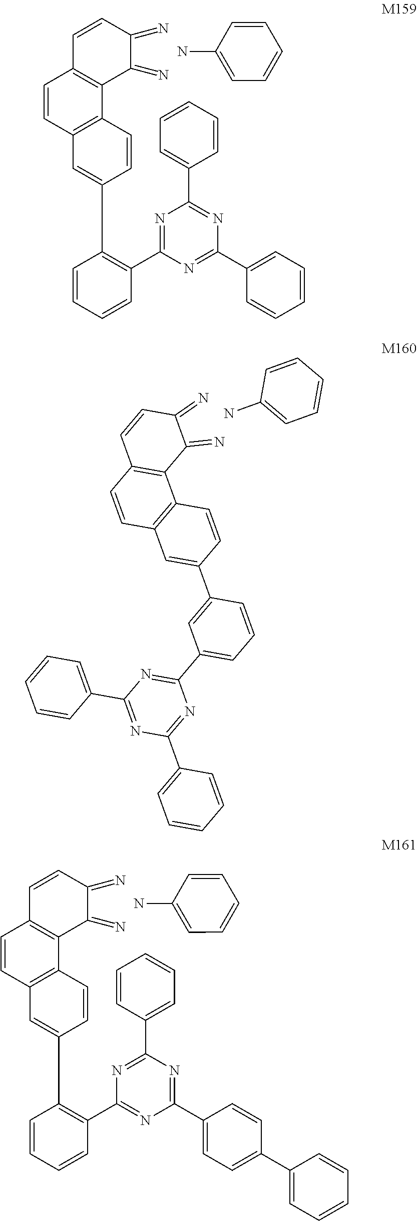

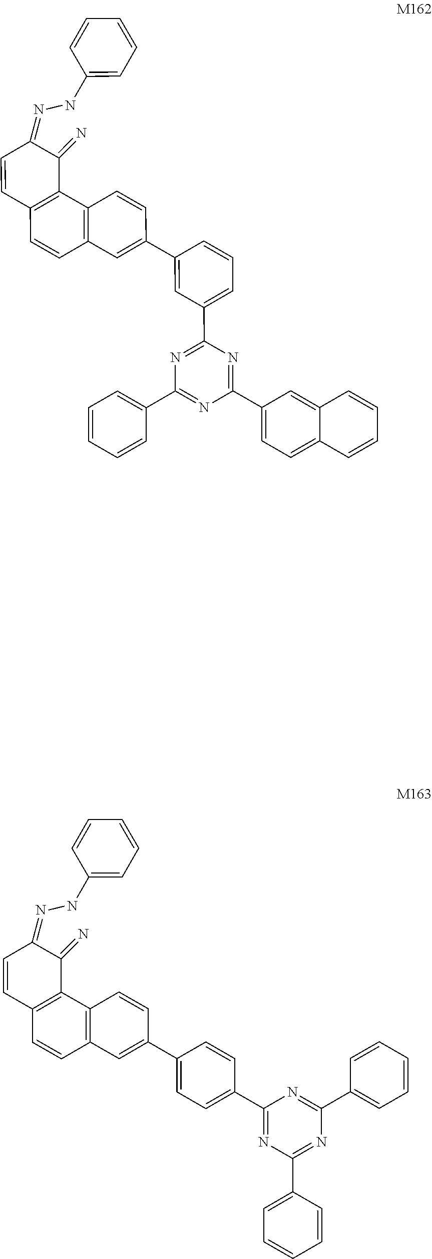 [SMILES: N=C1/C(=N\Nc2ccccc2)C=Cc2ccc3cc(-c4cccc(-c5nc(-c6ccccc6)nc(-c6ccc7ccccc7c6)n5)c4)ccc3c21 ; N=C1/C(=N\Nc2ccccc2)C=Cc2ccc3cc(-c4ccc(-c5nc(-c6ccccc6)nc(-c6ccccc6)n5)cc4)ccc3c21]

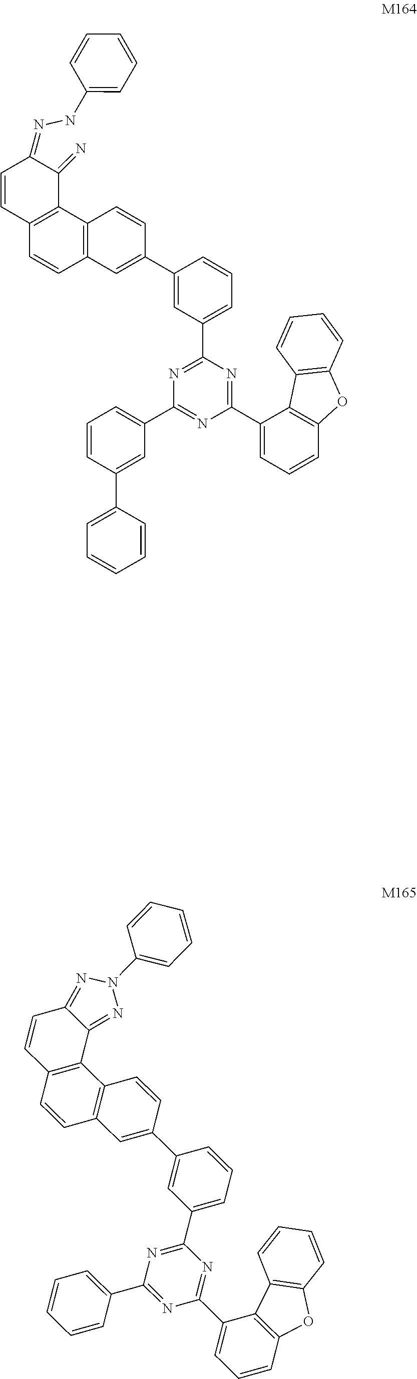 [SMILES: N=C1/C(=N\Nc2ccccc2)C=Cc2ccc3cc(-c4cccc(-c5nc(-c6cccc(-c7ccccc7)c6)nc(-c6cccc7oc8ccccc8c67)n5)c4)ccc3c21 ; c1ccc(-c2nc(-c3cccc(-c4ccc5c(ccc6ccc7nn(-c8ccccc8)nc7c65)c4)c3)nc(-c3cccc4oc5ccccc5c34)n2)cc1]

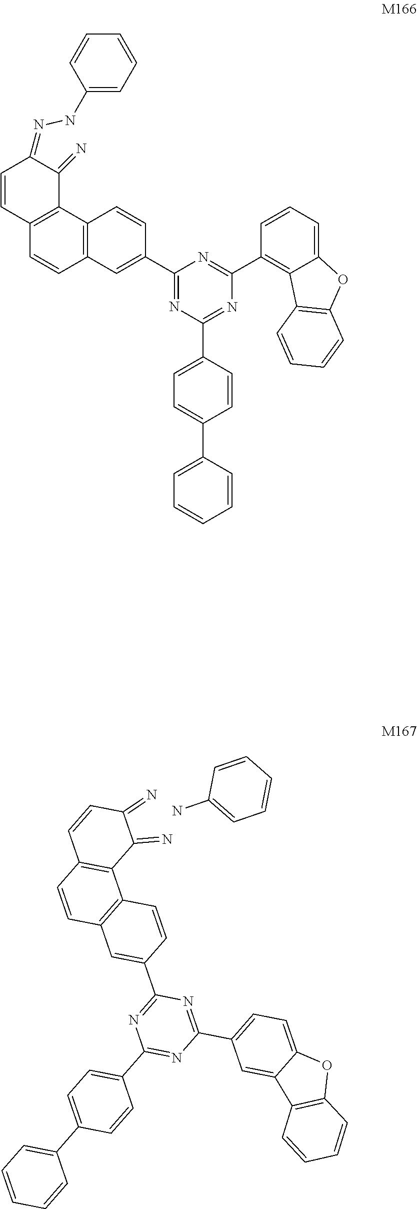

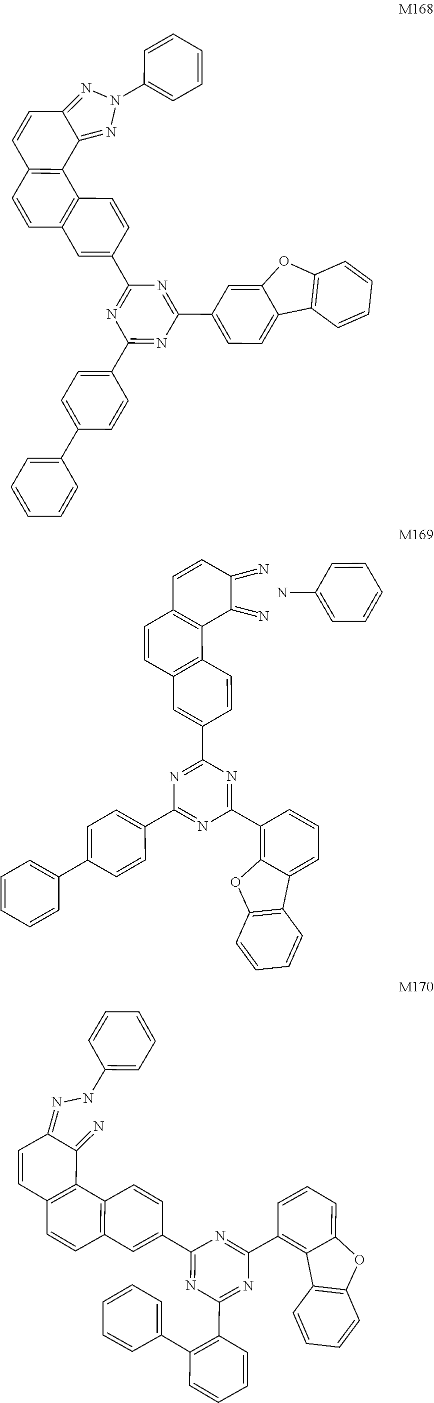

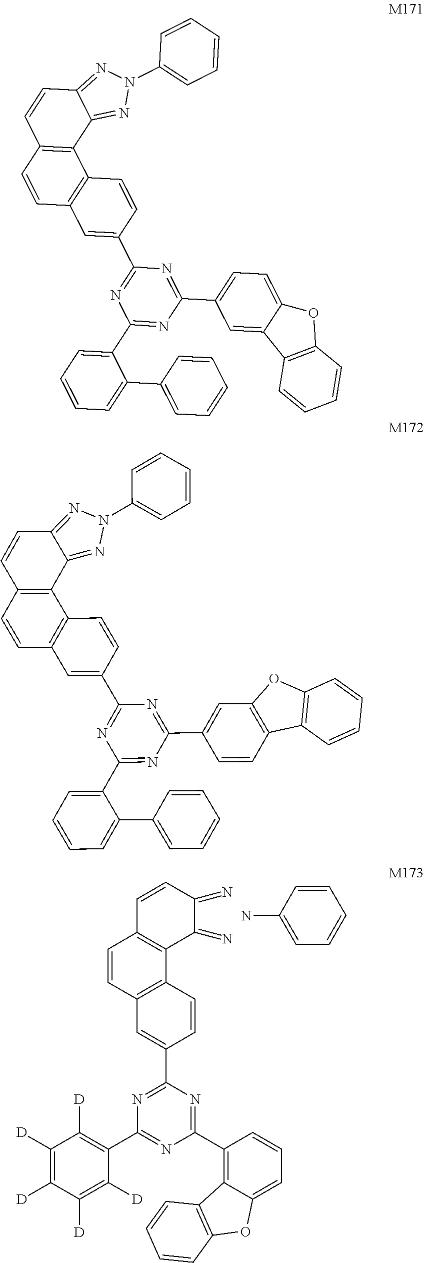

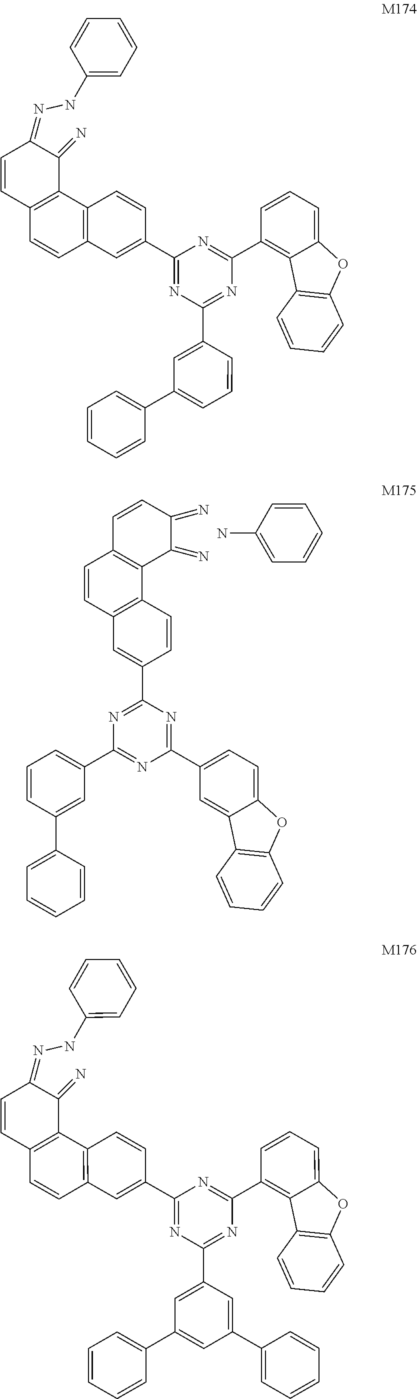

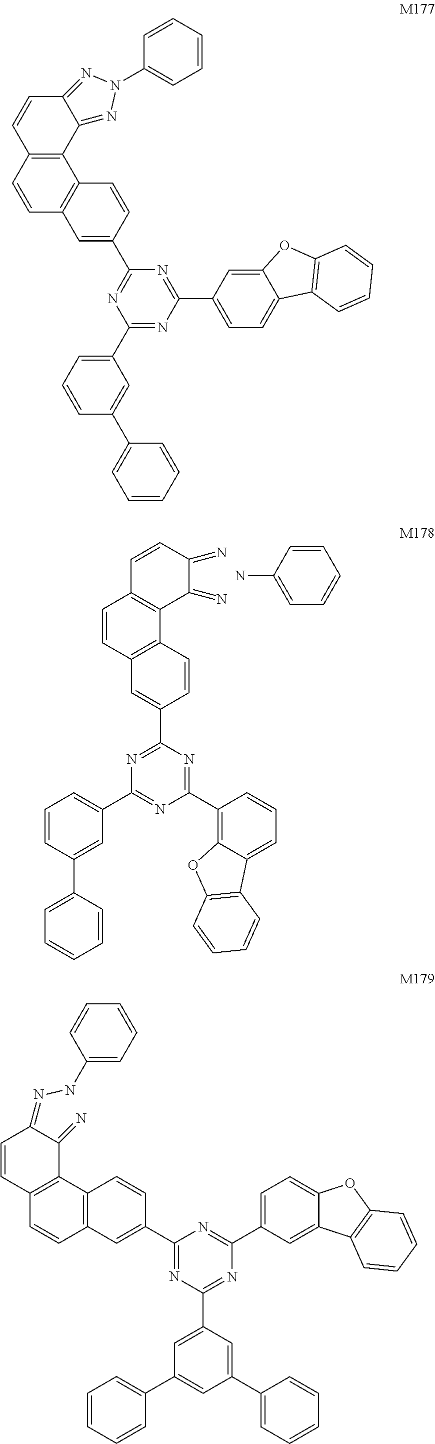

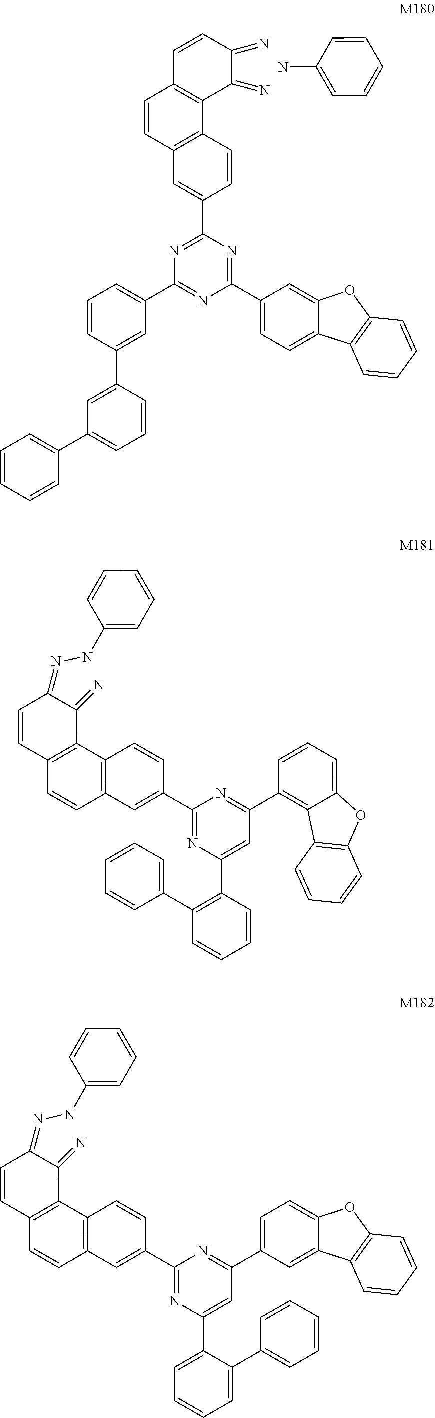

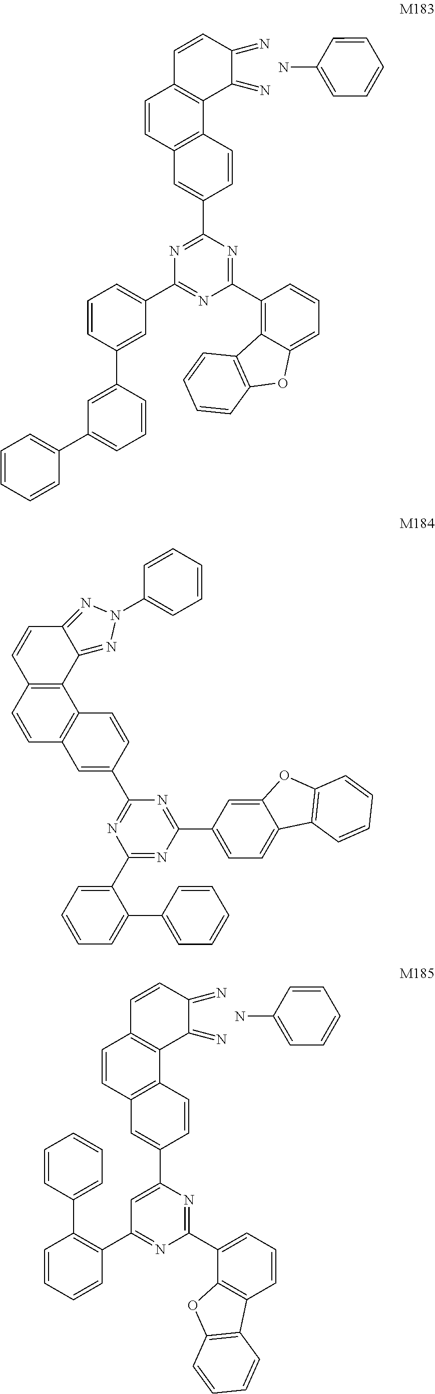

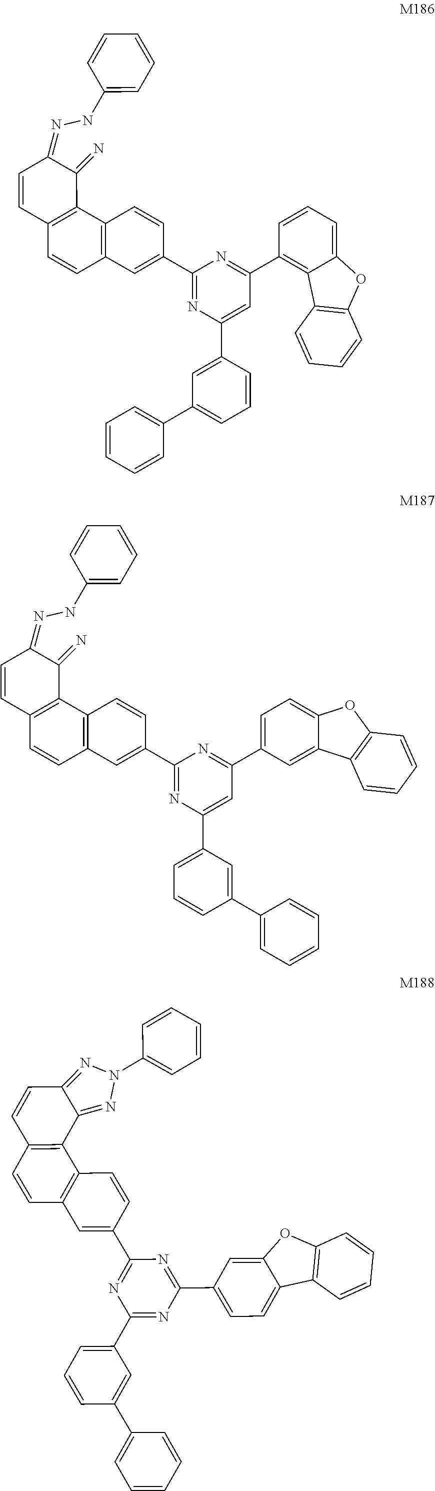 [SMILES: N=C1/C(=N\Nc2ccccc2)C=Cc2ccc3cc(-c4nc(-c5cccc(-c6ccccc6)c5)cc(-c5cccc6oc7ccccc7c56)n4)ccc3c21 ; N=C1/C(=N\Nc2ccccc2)C=Cc2ccc3cc(-c4nc(-c5cccc(-c6ccccc6)c5)cc(-c5ccc6oc7ccccc7c6c5)n4)ccc3c21 ; c1ccc(-c2cccc(-c3nc(-c4ccc5c(ccc6ccc7nn(-c8ccccc8)nc7c65)c4)nc(-c4ccc5c(c4)oc4ccccc45)n3)c2)cc1]

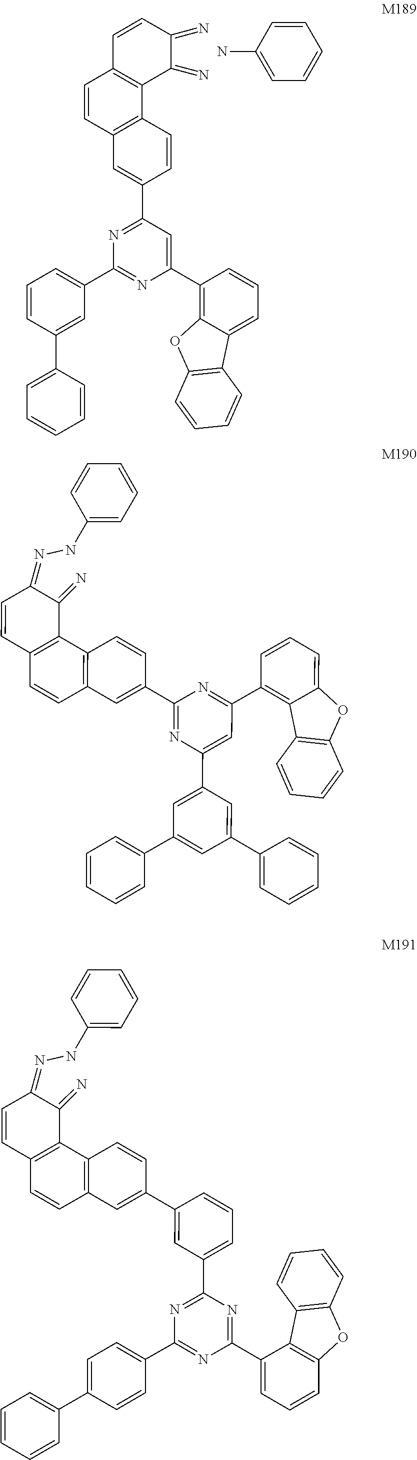

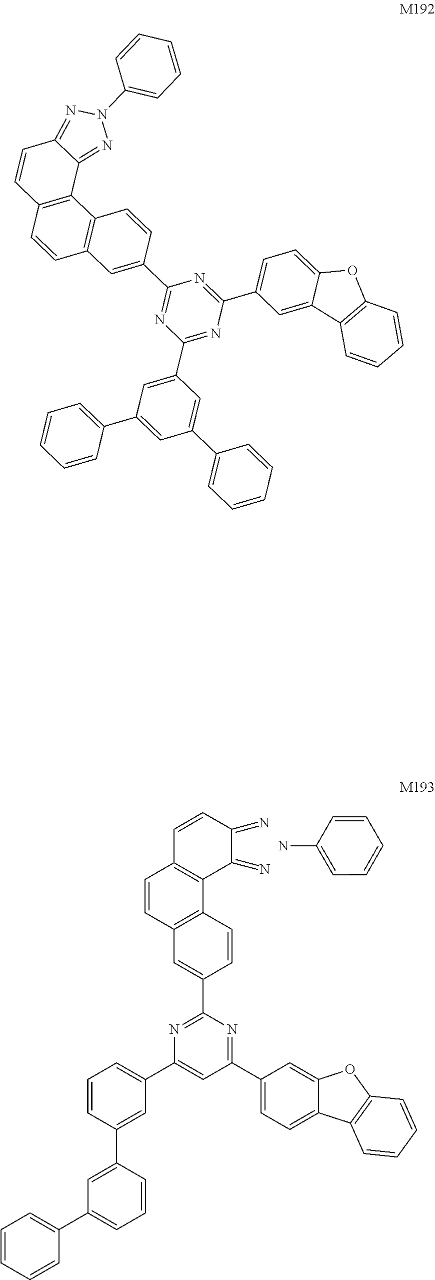

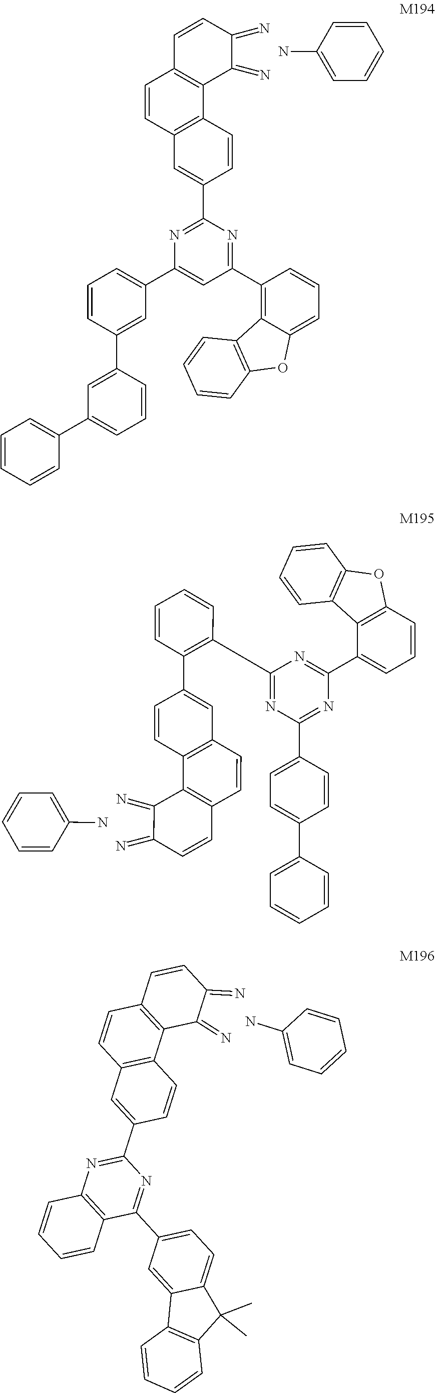

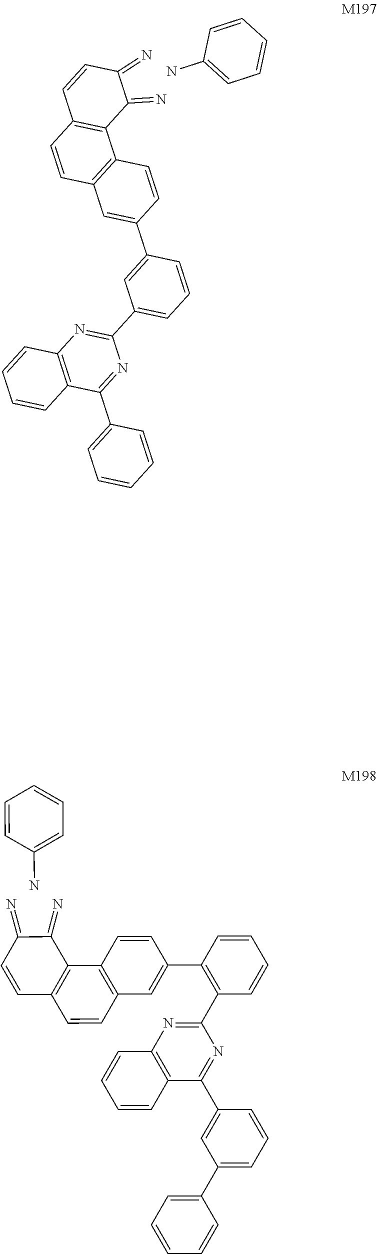

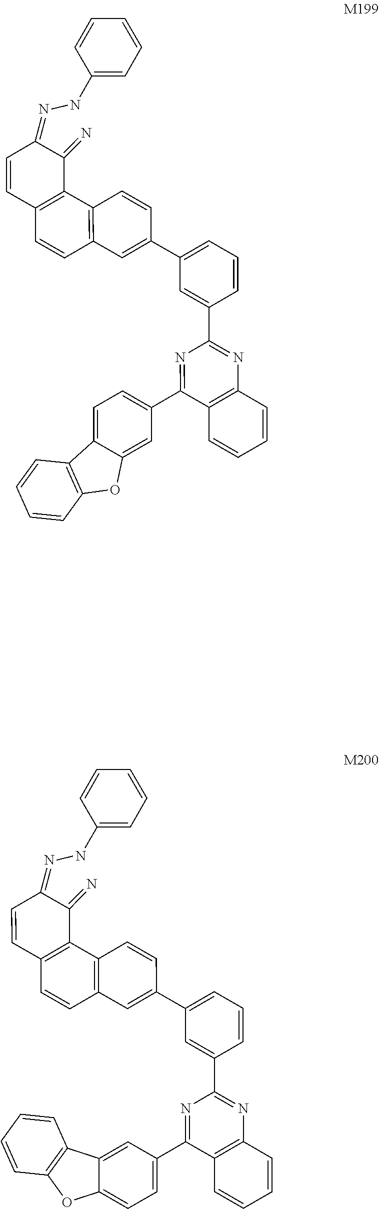 [SMILES: N=C1/C(=N\Nc2ccccc2)C=Cc2ccc3cc(-c4cccc(-c5nc(-c6ccc7c(c6)oc6ccccc67)c6ccccc6n5)c4)ccc3c21 ; N=C1/C(=N\Nc2ccccc2)C=Cc2ccc3cc(-c4cccc(-c5nc(-c6ccc7oc8ccccc8c7c6)c6ccccc6n5)c4)ccc3c21]

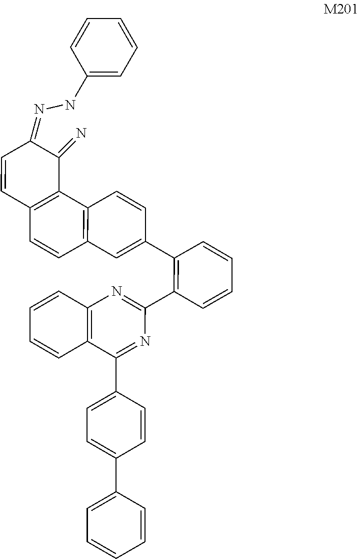 [SMILES: N=C1/C(=N\Nc2ccccc2)C=Cc2ccc3cc(-c4ccccc4-c4nc(-c5ccc(-c6ccccc6)cc5)c5ccccc5n4)ccc3c21]

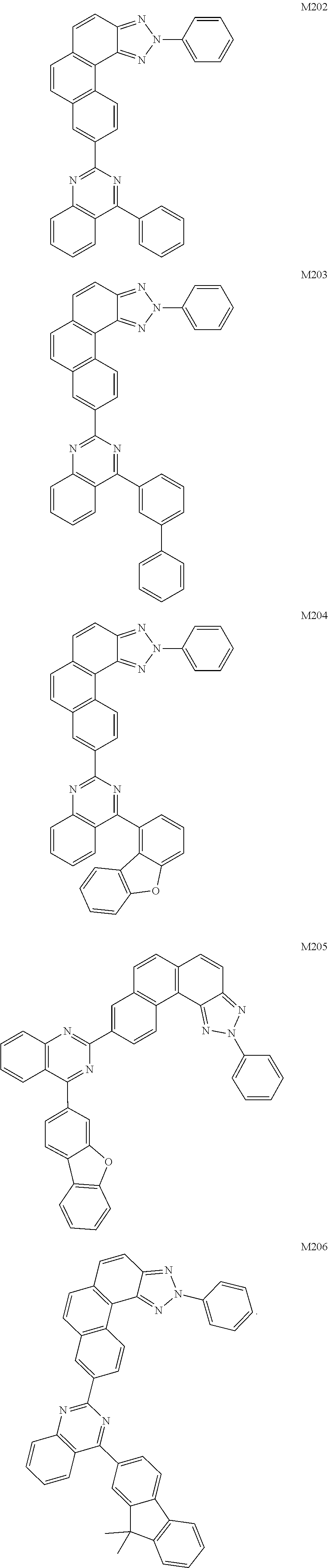

Preferably, the compound is a compound N having hole transport properties.

Preferably, the compound is any-one of compounds N1 to N115:

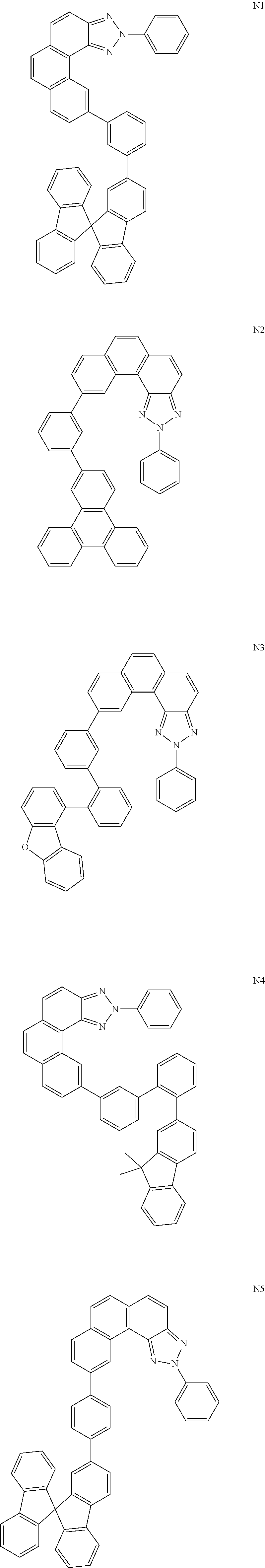

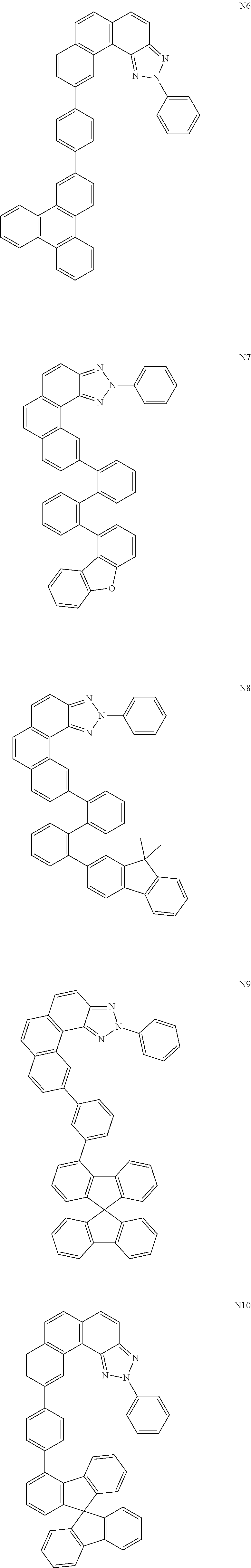

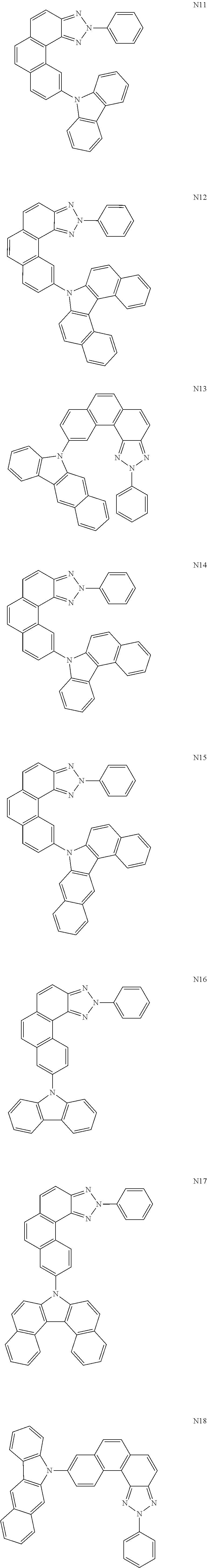

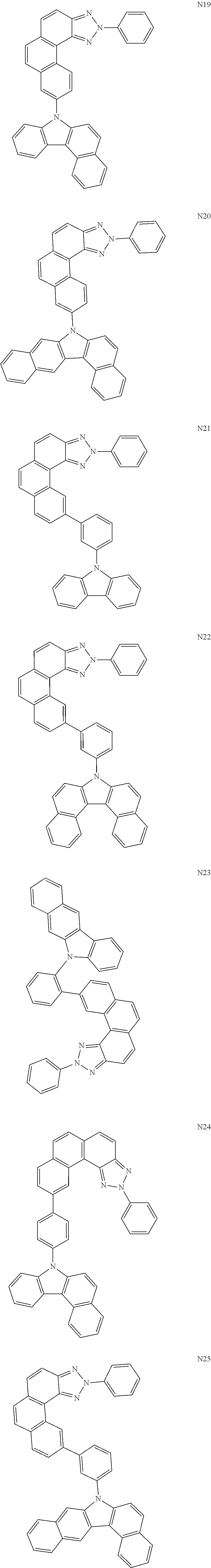

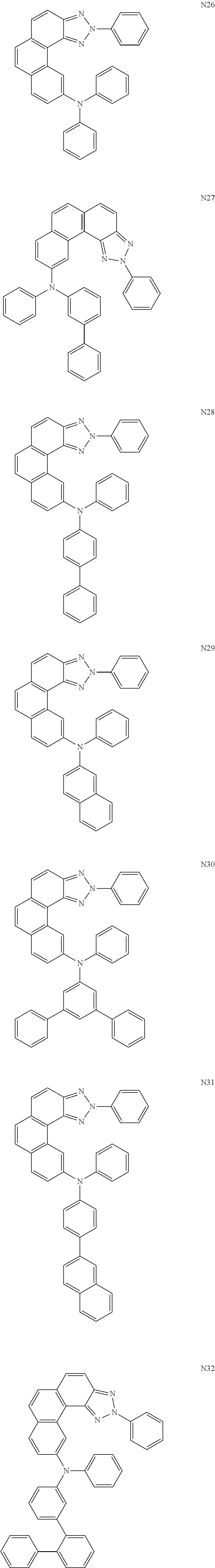

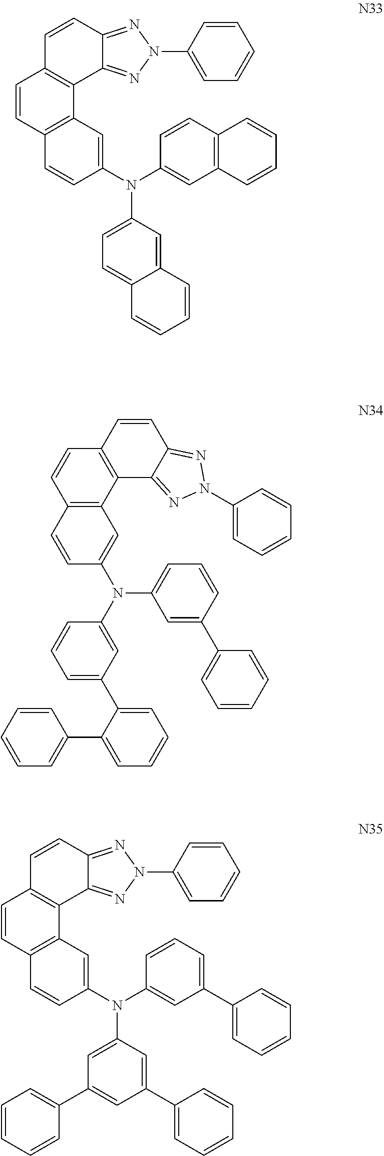

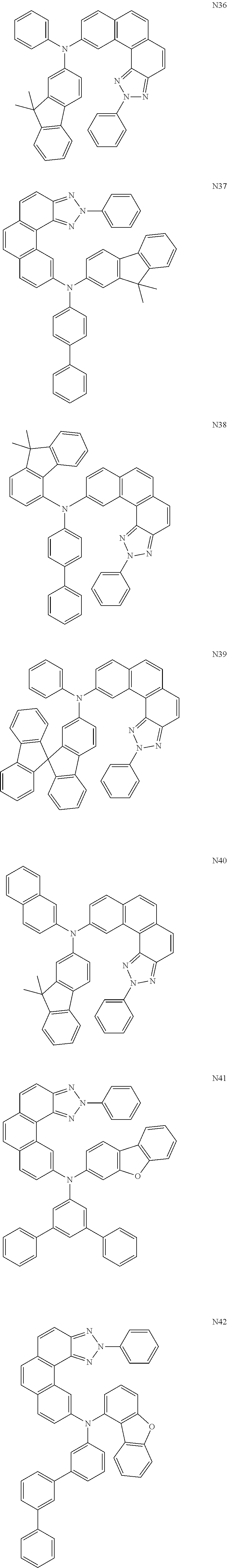

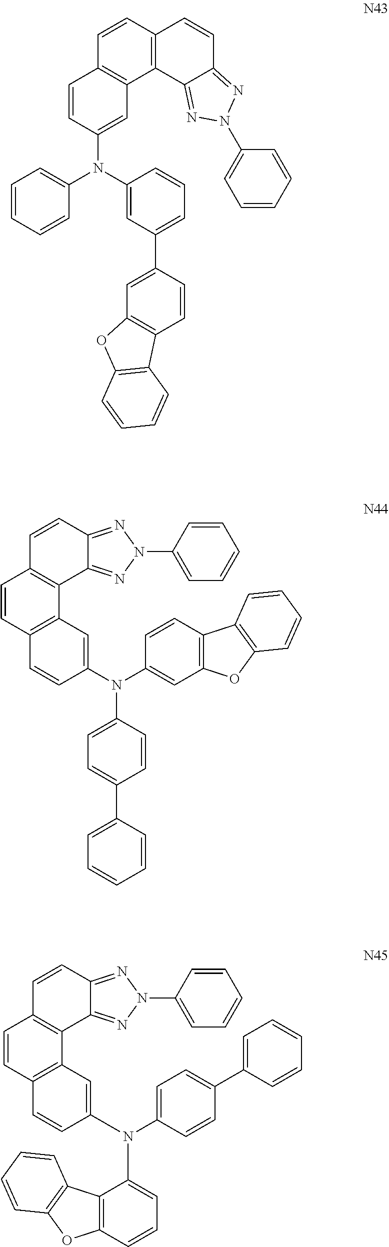

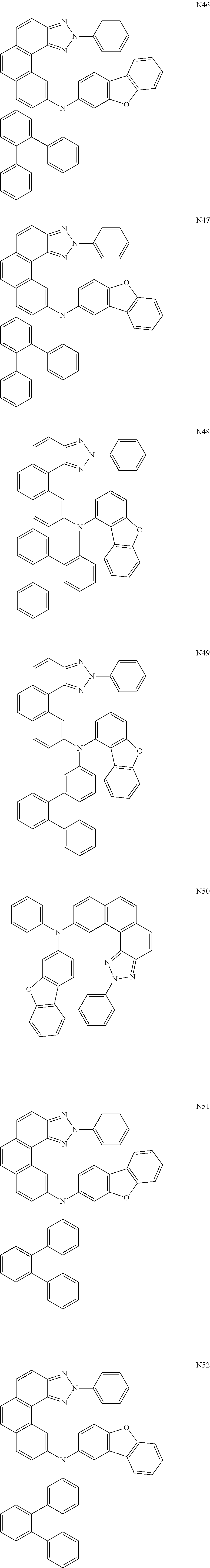

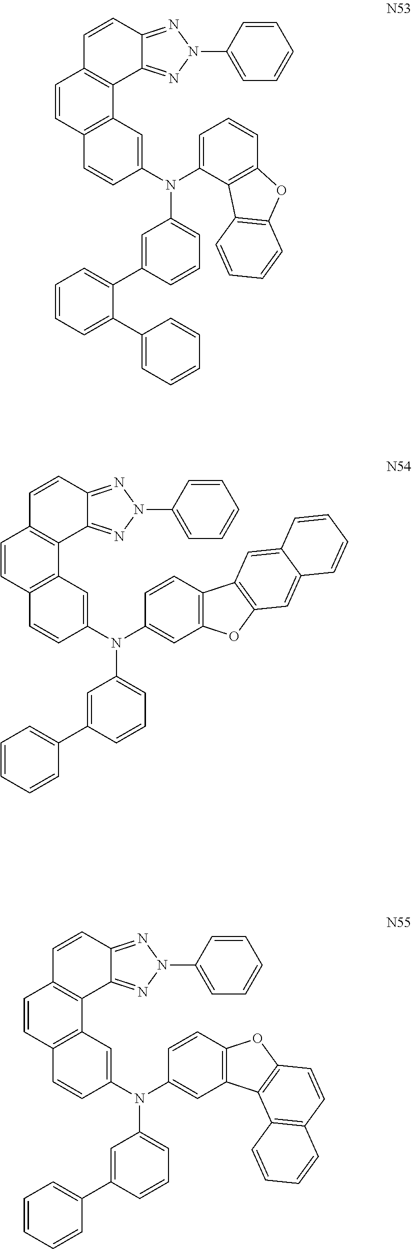

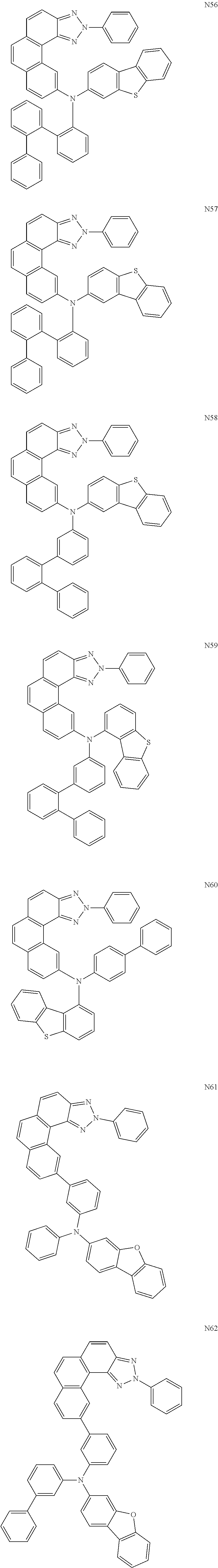

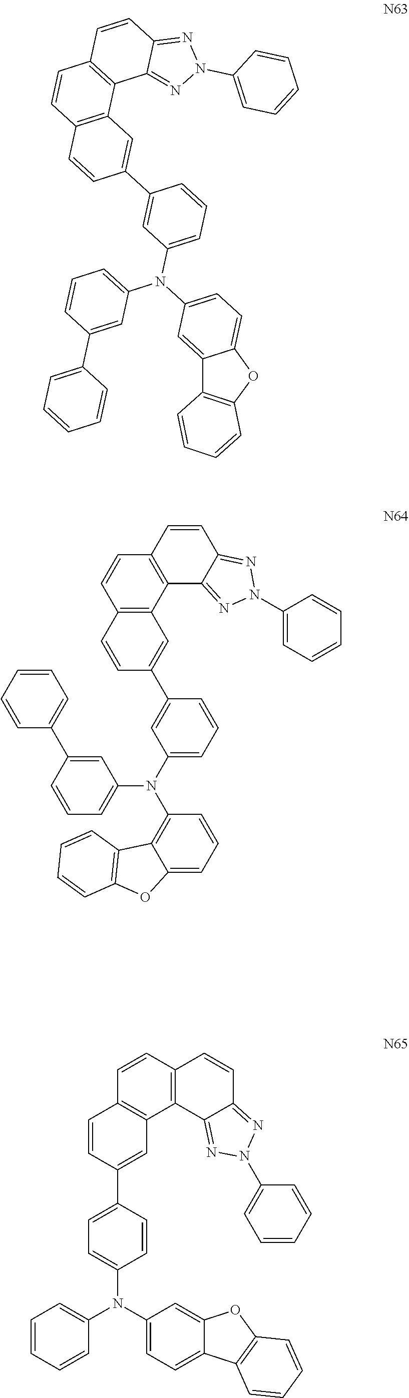

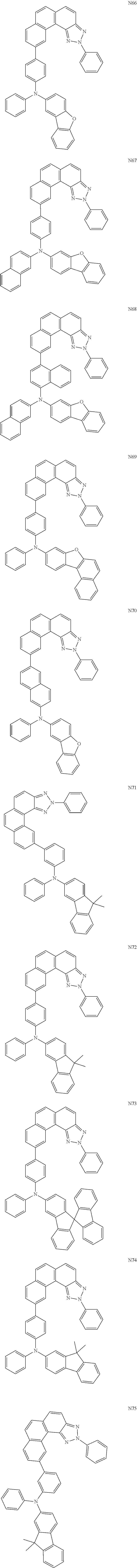

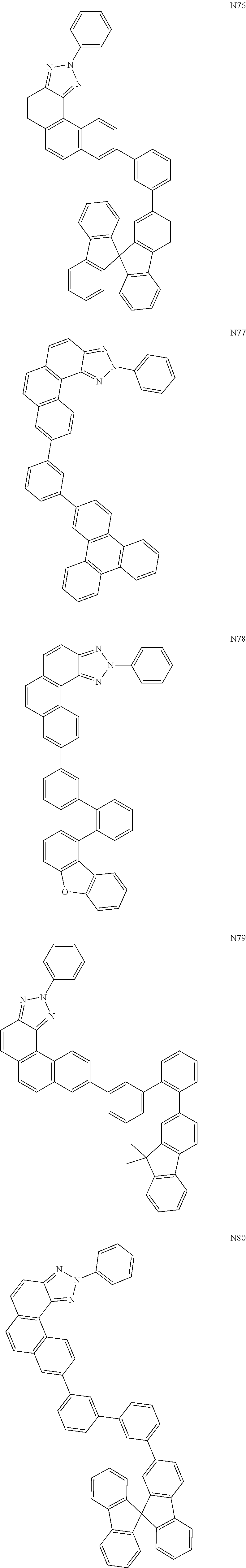

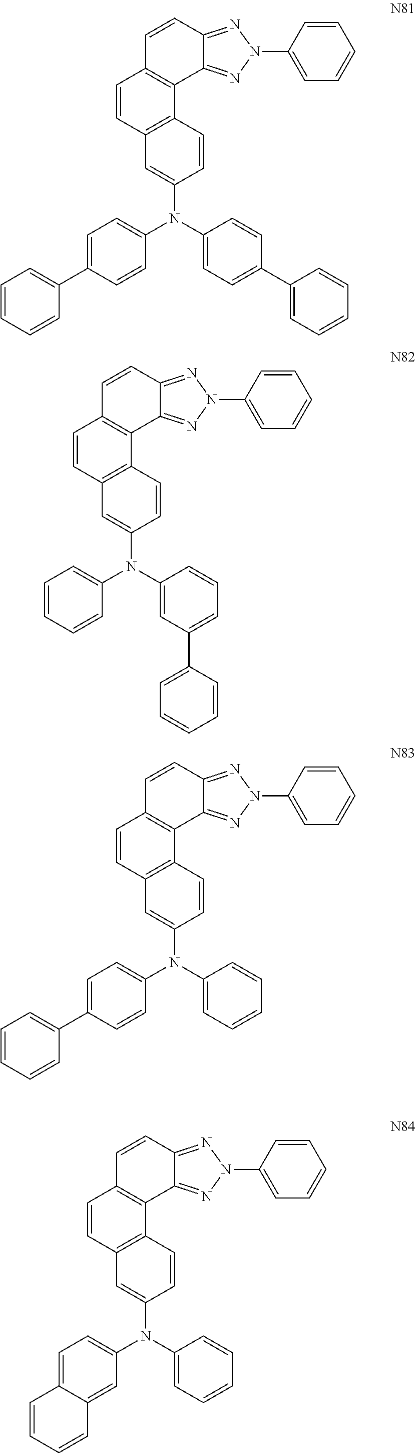

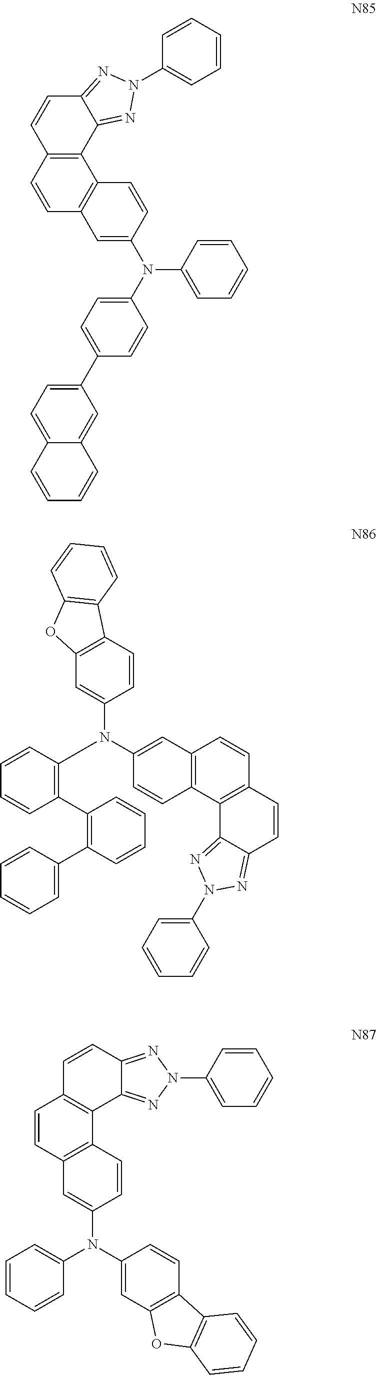

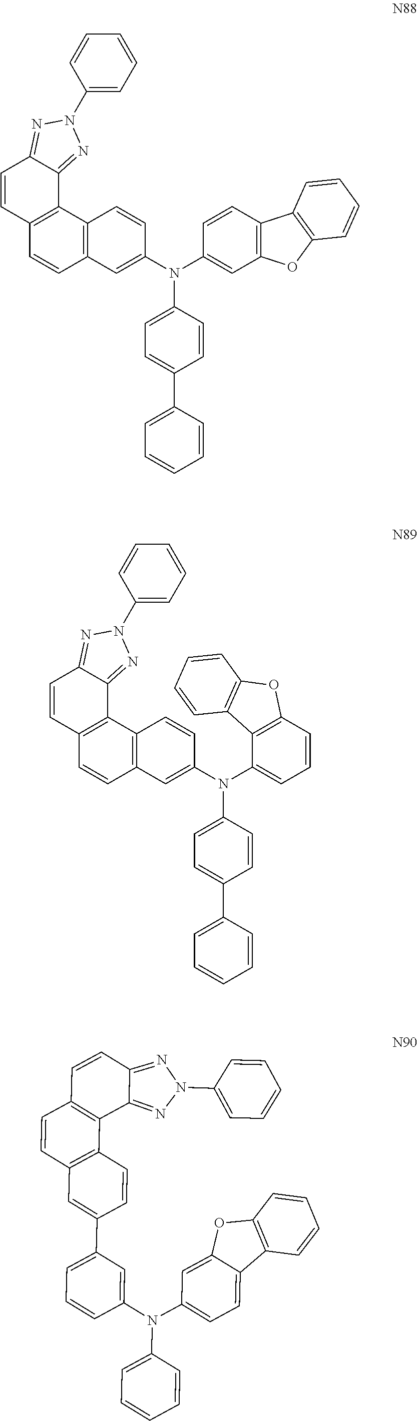

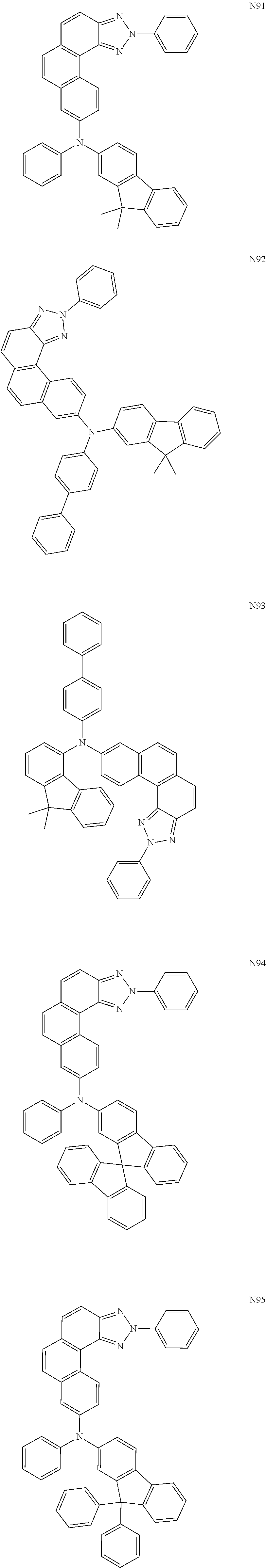

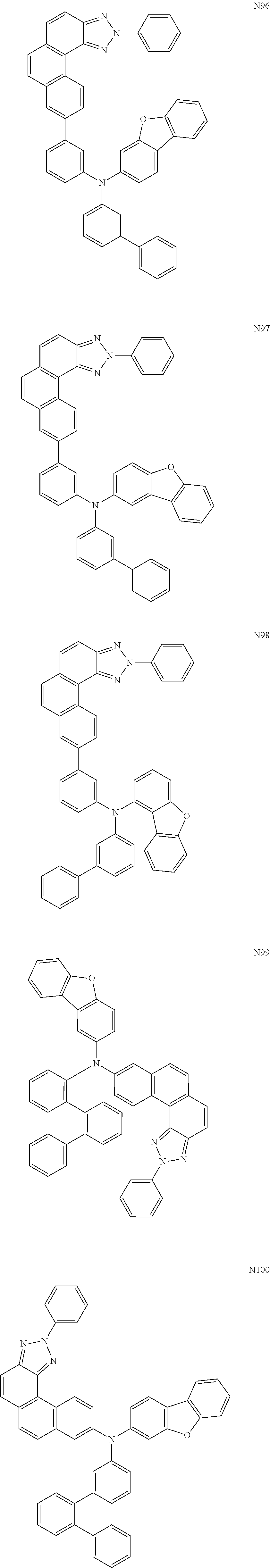

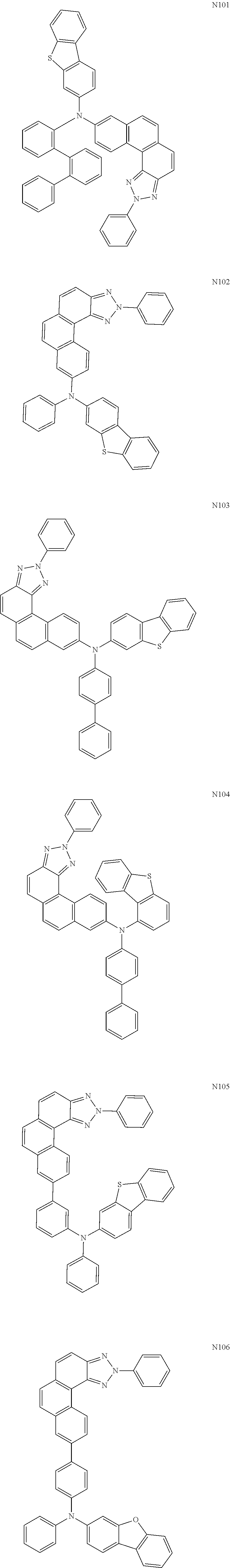

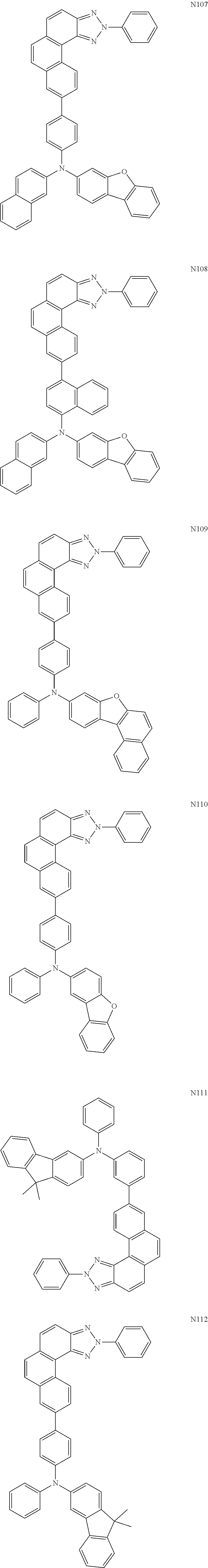

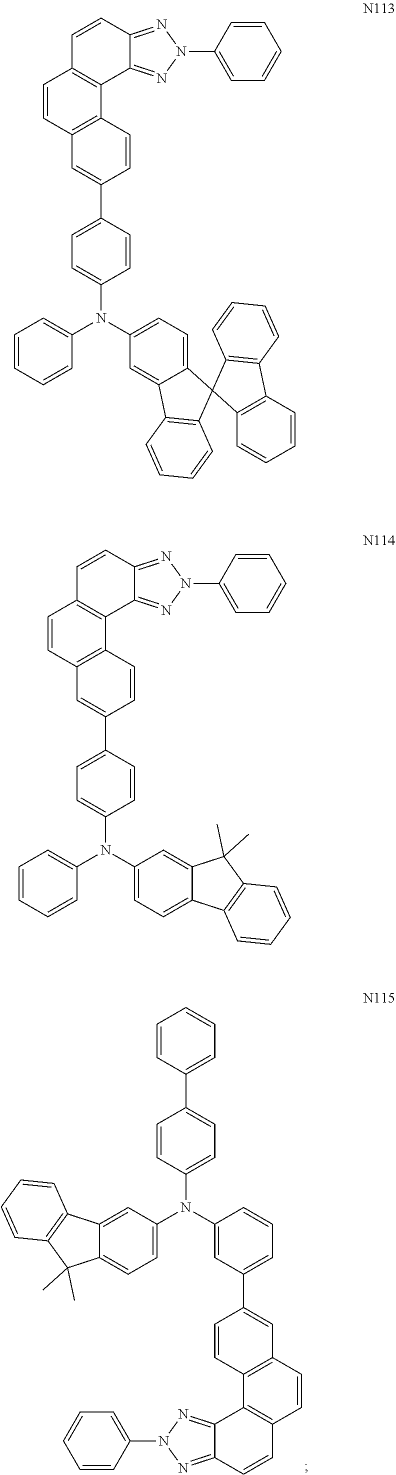

wherein D represents deuterium.

In another aspect, the present invention provides an organic electroluminescence material, which comprises least one of the above-mentioned compounds.

Preferably, the organic electroluminescence material comprises a compound of Formula (1) in which at least one of Ar¹ to Ar⁴ is selected from a group represented by Formula a, and a compound of Formula (1) in which at least one of Ar¹ to Ar⁴ is selected from a group represented by Formula b.

Preferably, the organic electroluminescence material comprises a compound of Formula (1) in which one of Ar¹ to Ar⁴ is selected from a group represented by Formula a, and a compound of Formula (1) in which one of Ar¹ to Ar⁴ is selected from a group represented by Formula b.

Preferably, the organic electroluminescence material comprises a compound of Formula (1) in which Ar² is selected from a group represented by Formula a, and a compound of Formula (1) in which Ar² is selected from a group represented by Formula b.

Preferably, the organic electroluminescence material comprises a compound of Formula (1) in which Ar³ is selected from a group represented by Formula a, and a compound of Formula (1) in which Ar³ is selected from a group represented by Formula b.

Preferably, the organic electroluminescence material comprises a compound of Formula (1) in which at least one of Ar¹ to Ar⁴ is selected from a group represented by Formula a, and a compound of Formula (1) in which at least one of Ar¹ to Ar⁴ is selected from a group represented by Formula c.

Preferably, the organic electroluminescence material comprises a compound of Formula (1) in which one of Ar¹ to Ar⁴ is selected from a group represented by Formula a, and a compound of Formula (1) in which one of Ar¹ to Ar⁴ is selected from a group represented by Formula c.

Preferably, the organic electroluminescence material comprises a compound of Formula (1) in which Ar² is selected from a group represented by Formula a, and a compound of Formula (1) in which Ar² is selected from a group represented by Formula c.

Preferably, the organic electroluminescence material comprises a compound of Formula (1) in which Ar³ is selected from a group represented by Formula a, and a compound of Formula (1) in which Ar³ is selected from a group represented by Formula c.

Preferably, the organic electroluminescence material comprises a compound of Formula (1) in which at least one of Ar¹ to Ar⁴ is selected from a group represented by Formula a, and a compound of Formula (1) in which at least one of Ar¹ to Ar⁴ is selected from a phenyl group, a naphthyl group, a biphenylyl group, a phenanthryl group, a fluoranthenyl group, a triphenylenylene group, a dimethylfluorenyl group, a diphenylfluorenyl group, a spiro-bifluorenyl group, a benzodimethylfluorenyl group, a benzodiphenylfluorenyl group, a benzo-spiro-bifluorenyl group, a dibenzofuryl group, a benzonaphthofuryl group, a benzonaphthothiophenyl group, and a dibenzothiophenyl group, each of which is substituted or unsubstituted.

Preferably, the organic electroluminescence material comprises a compound of Formula (1) in which one of Ar¹ to Ar⁴ is selected from a group represented by Formula a, and a compound of Formula (1) in which one of Ar¹ to Ar⁴ is selected from a phenyl group, a naphthyl group, a biphenylyl group, a phenanthryl group, a fluoranthenyl group, a triphenylenylene group, a dimethylfluorenyl group, a diphenylfluorenyl group, a spiro-bifluorenyl group, a benzodimethylfluorenyl group, a benzodiphenylfluorenyl group, a benzo-spiro-bifluorenyl group, a dibenzofuryl group, a benzonaphthofuryl group, a benzonaphthothiophenyl group, and a dibenzothiophenyl group, each of which is substituted or unsubstituted.

Preferably, the organic electroluminescence material comprises a compound of Formula (1) in which Ar² is selected from a group represented by Formula a, and a compound of Formula (1) in which Ar² is selected from a phenyl group, a naphthyl group, a biphenylyl group, a phenanthryl group, a fluoranthenyl group, a triphenylenylene group, a dimethylfluorenyl group, a diphenylfluorenyl group, a spiro-bifluorenyl group, a benzodimethylfluorenyl group, a benzodiphenylfluorenyl group, a benzo-spiro-bifluorenyl group, a dibenzofuryl group, a benzonaphthofuryl group, a benzonaphthothiophenyl group, and a dibenzothiophenyl group, each of which is substituted or unsubstituted.

Preferably, the organic electroluminescence material comprises a compound of Formula (1) in which Ar³ is selected from a group represented by Formula a, and a compound of Formula (1) in which Ar³ is selected from a phenyl group, a naphthyl group, a biphenylyl group, a phenanthryl group, a fluoranthenyl group, a triphenylenylene group, a dimethylfluorenyl group, a diphenylfluorenyl group, a spiro-bifluorenyl group, a benzodimethylfluorenyl group, a benzodiphenylfluorenyl group, a benzo-spiro-bifluorenyl group, a dibenzofuryl group, a benzonaphthofuryl group, a benzonaphthothiophenyl group, and a dibenzothiophenyl group, each of which is substituted or unsubstituted.

Preferably, the organic electroluminescence material comprises at least one compound M having hole transport properties and at least one compound N having electron transport properties.

Preferably, the organic electroluminescence material comprises at least one of compounds M1 to M206 and at least one of compounds N1 to N115.

As used in the present invention, the term “halogen” may comprise fluorine, chlorine, bromine or iodine; preferably fluorine.

As used in the present invention, the term “C1-C30 alkyl group” indicates a monovalent substituent derived from a straight or branched saturated hydrocarbon having 1 to 30 carbon atoms, for example, it comprises, but is not limited to a methyl group, an ethyl group, a propyl group, an isobutyl group, a sec-butyl group, a tert-butyl group, a pentyl group, an isopentyl group, or a hexyl.

As used in the present invention, the term “C3-C30 cycloalkyl group” indicates a group derived from a monocyclic hydrocarbon or a multicyclic hydrocarbon having 1 to 30 carbon atoms on the main chain, and the cycloalkyl group may comprise cyclopropyl, cyclobutyl, adamantyl group, or the like.

In the present invention, the aryl group and arylene group respectively comprise a monocyclic, a multicyclic or a fused cyclic aryl group and arylene group, in which the rings may be interrupted by a short non-aromatic unit, and they may comprise a spiro-structure. The aryl group and arylene group of the present invention comprise, but are not limited to, a phenyl group, a biphenylyl group, a terphenylyl group, a naphthyl group, a phenanthryl group, an anthryl group, a fluorenyl group, a spiro-bifluorenyl group, or the like.

In the present invention, heteroaryl group and heteroarylene group respectively comprise a monocyclic, a multicyclic or a fused cyclic heteroaryl group and heteroarylene group, in which the rings may be interrupted by a short non-aromatic unit, and the hetero atom comprises nitrogen, oxygen or sulfur. The heteroaryl group and heteroarylene group of the present invention comprise, but are not limited to, a furyl group, a thiophenyl group, a pyrrolyl group, an imidazolyl group, a pyrazolyl group, a thiazolyl group, a thiadiazolyl group, an isothiazolyl group, an isoxazolyl group, an oxazolyl group, an oxadizolyl group, a triazinyl group, a tetrazinyl group, a triazolyl group, a tetrazolyl group, a furazanyl group, a pyridyl group, a pyrazinyl group, a pyrimidinyl group, a pyridazinyl group, a benzofuryl group, a benzothiophenyl group, an isobenzofuryl group, a dibenzofuryl group, a dibenzothiophenyl group, a benzimidazolyl group, a benzothiazolyl group, a benzisothiazolyl group, a benzisoxazolyl group, a benzoxazolyl group, an isoindolyl group, an indolyl group, an indazolyl group, a benzothiadiazolyl group, a quinolyl group, an isoquinolyl group, a cinnolinyl group, a quinazolinyl group, a quinoxalinyl group, a carbazolyl group, a phenoxazinyl group, a phenothiazinyl group, a phenanthridinyl group, a 1,3-benzodioxolyl group, a dihydroacridinyl group, or derivatives thereof.

Preferably, the aryl group is selected from a phenyl group, a biphenylyl group, a terphenylyl group, a naphthyl group, an anthryl group, a phenanthryl group, a 9,9′-dimethylfluorenyl group, a 9,9′-diphenylfluorenyl group and a spiro-bifluorenyl group.

Preferably, the heteroaryl group is selected from a dibenzofuryl group, a dibenzothiophenyl group, a carbazolyl group, a triazinyl group, a pyridyl group, a pyrimidinyl group, an imidazolyl group, an oxazolyl group, a thiazolyl group, a benzimidazolyl group, a benzoxazolyl group, a benzothiazolyl, a naphthimidazolyl group, a naphthoxazolyl group, a naphthothiazolyl group, a phenanthrimidazolyl group, a phenanthroxazolyl group, a phenanthrothiazolyl group, a quinoxalinyl group, a quinazolinyl group, an indolocarbazolyl group, an indolofluorenyl group, a benzothienopyrazinyl group, a benzothienopyrimidinyl group, a benzofuropyrazinyl group, a benzofuropyrimidinyl group, an indolopyrazinyl group, an indolopyrimidinyl group, an indenopyrazinyl group, an indenopyrimidinyl group, a spiro[fluorene-9,1′-indene]-pyrazinyl group, a spiro[fluorene-9,1′-indene]-pyrimidinyl group, benzofurocarbazolyl and benzothienocarbazolyl.

As used in the present invention, the term “C6-C30 aryloxy group” indicates a monovalent substituent represented by RO—, wherein R represents an aryl group having 6 to 30 carbon atoms. Examples of such aryloxy group comprise, but are not limited to, a phenoxy group, a naphthyloxy group, a diphenoxy group, or the like.

As used in the present invention, the term “C1-C30 alkoxy group” indicates a monovalent substituent represented by R′O—, wherein R′ represents an alkyl group having 1 to 30 carbon atoms.

As used in the present invention, the term “substituted” indicates a hydrogen atom comprised in a compound is replaced by another substituent. The position of substitution is not specifically limited, provided that the hydrogen at the position can be replaced by the substituent. When two or more substituents are simultaneously present, the two or more substituents can be the same or different.

As used in the present invention, unless otherwise specified, the hydrogen atom comprises protium, deuterium or tritium.

In the present invention, “adjacent two groups joined to form a ring” indicates that 2 substituents at adjacent positions on the same ring or adjacent rings can be joined to a ring by chemical bonding. The specific way to form a ring in the present invention is not limited (for example, joined via a single bond, joined via a benzene ring, joined via a naphthalene ring, fused via

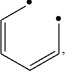

fused via

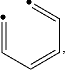

fused

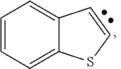

fused via

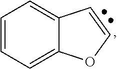

fused via

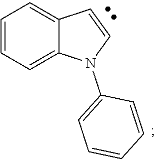 [SMILES: c1ccc(-n2ccc3ccccc32)cc1]

wherein the [symbol] represents fusion positions). In the same description present hereinafter, it has the same meaning.

In the present invention, when the range of carbon atom number is limited in the definition of a functional group, the functional group may have a carbon atom number of any integer in the limited range. For example, a C6-C60 aryl group represents an aryl group that may give a carbon number of any one integer comprised in the range of 6 to 60, such as 6, 8, 10, 15, 20, 30, 35, 40, 45, 50, 55 or 60, etc.

In the present invention, the organic electroluminescence material is prepared by a synthesis route shown as below:

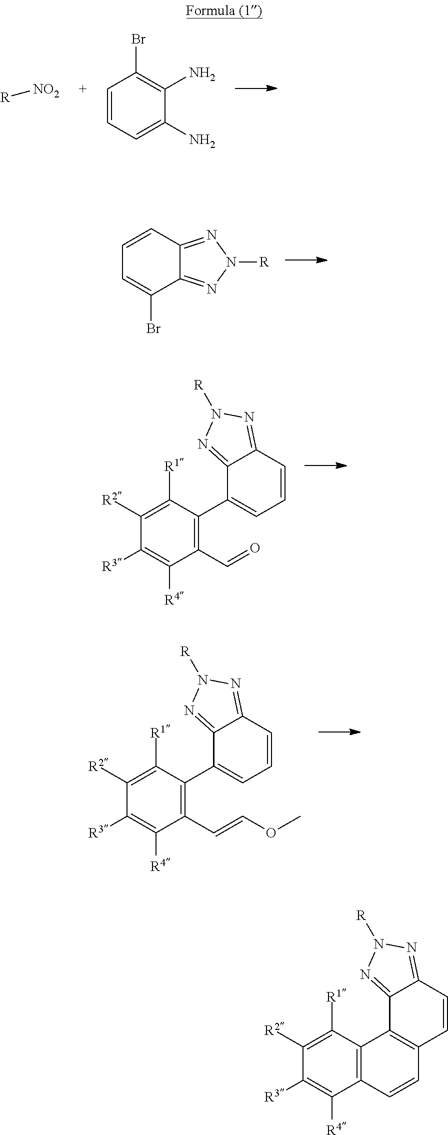

wherein at least one of R^{1″}, R^{2″}, R^{3″} and R^{4″} is chlorine; and the R^{1″}, R^{2″}, R^{3″} or R^{4″} which is not chlorine has the same definition with the corresponding R¹, R², R³ or R⁴.

The synthesis equation of the compound of Formula (1) formed by connecting Formula (1″) and Formula b or Formula c via chemical bonding is shown as below:

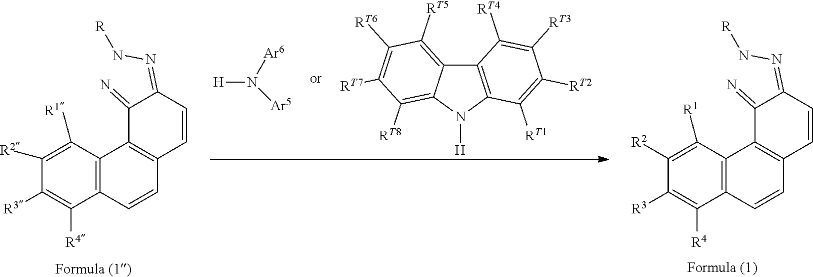

The synthesis equation of the compound of Formula (1) formed by connecting Formula (1″) and Formula a via chemical bonding is shown as below:

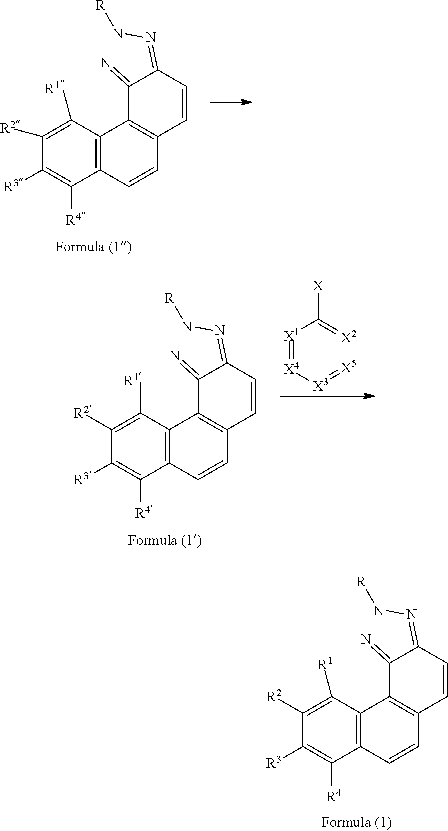

wherein at least one of R^{1′}, R^{2′}, R^{3′} and R^{4′} is a pinacolato group

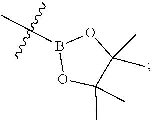 [SMILES: *B1OC(C)(C)C(C)(C)O1]

and
the R^{1′}, R^{2′}, R^{3′} or R^{4′} which is not a pinacolato group has the same definition with the corresponding R¹, R², R³ or R⁴; and X is halogen.

Preferably, the organic electroluminescence material comprises at least one compound M having hole transport properties and at least one compound N having electron transport properties.

Preferably, the organic electroluminescence material comprises at least one of compounds M1 to M206 and at least one of compounds N1 to N115.

The combination of the hole- and electron-transport compounds of the present invention achieves the transport balance of holes and electrons, and this makes OLEDs have a higher emitting efficiency and a longer service life.

In another aspect, the present invention provides an organic electroluminescence element, and the organic electroluminescence element comprises a first electrode, a second electrode, and an organic layer disposed between the first electrode and the second electrode; and the organic layer is made of a material comprising the above-mentioned compound.

Preferably, the organic layer is made of a material comprising the above-mentioned organic electroluminescence material.

Preferably, the organic layer comprises an emitting layer, and the emitting layer comprises the above-mentioned compound.

Preferably, the organic layer comprises an emitting layer, and the emitting layer comprises the above-mentioned organic electroluminescence material.

Preferably, the emitting layer material further comprises a coordination complex of a transition metal.

Preferably, the emitting layer further comprises a coordination complex comprising Ir or Pt.

In another aspect, the present invention provides an electronic device, comprising the organic electroluminescence element.

In the present invention, the electronic device can be applied to optoelectronics, medicine, biotechnology, an optical fiber, a lighting equipment, an electrophotographic photoreceptor, a photoelectric transducer, an organic solar cell, a light-emitting element, an organic light-emitting field-effect transistor, an image sensor or a dye laser.

Compared to the existing technology, the present invention has the following advantages:

When the compound of the present invention is used as an organic electroluminescence material, it makes the element have a lower driving voltage, a higher current efficiency and a longer service life.

The compound of the present invention can obviously increase the charge carrier injection efficiency. As a multi-component host material (i.e., with a compound M having hole transport properties and a compound N having electron transport properties), it can effectively reduce the energy level difference between layers, balance the electron- and hole-transport efficiency, increase the efficiency of the organic electroluminescence device, and prolong the service life of the organic electroluminescence device.

### DETAILED DESCRIPTION OF THE PREFERRED EMBODIMENTS

Specific embodiments are further illustrated by the following examples to demonstrate the technical approaches of the present invention. Those skilled in the art should understand that the illustrative examples are helpful to understand the present invention; however, they should not be construed as being limiting to the scope of the present invention.

### Preparation Examples of Compound of the Present Invention

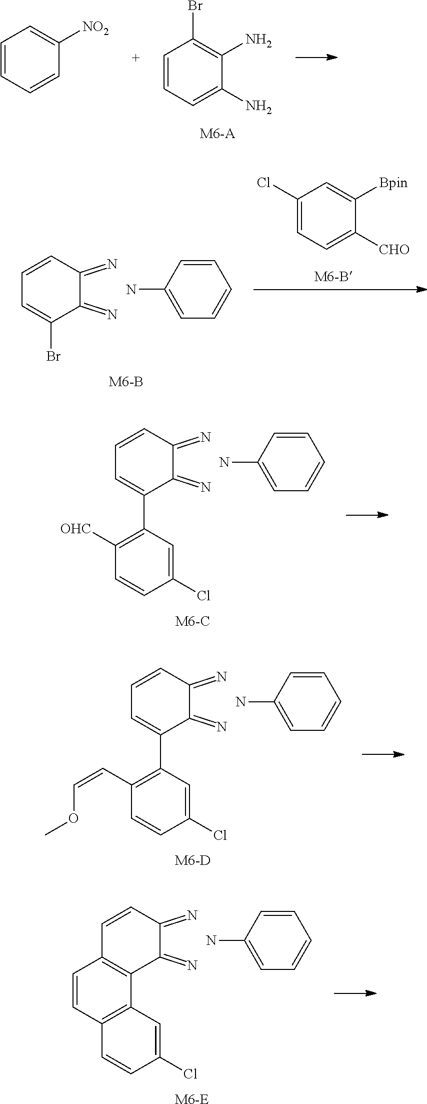

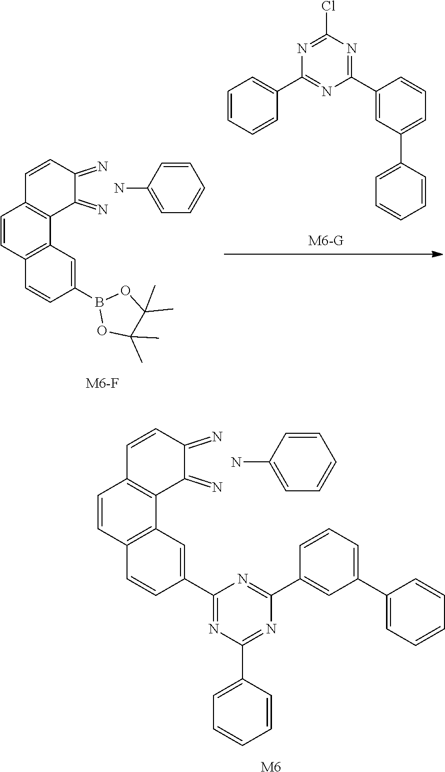

Synthesis of M6-B: In a three-necked bottle of 25 milliliters (mL), M6-A (10 millimoles (mmol)), nitrobenzene (10 mmol), potassium hydroxide (22 mmol), copper(I) thiocyanate (1 mmol) and anhydrous tetrahydrofuran (10 mL) were added, nitrogen gas was purged for three times, and heated to 90° C. under nitrogen gas protection to react for 48 hours (h). After the reaction ended, the reaction mixture was quenched by water, the reaction system was extracted by ethyl acetate, and the organic solvent was removed by rotary evaporation to give a crude product. The crude product was purified by column chromatography (ethyl acetate:n-hexane=1:50 (volume ratio)), to obtain M6-B (1.34 g, 49% yield).

Synthesis of M6-B′: In a three-necked bottle of 50 mL, 2-bromo-4-chlorobenzaldehyde (10 mmol), bis(pinacolato)diboron (12 mmol), potassium acetate (100 mmol), [1,1′-bis(diphenylphosphino)ferrocene]palladium(II) dichloride (0.2 mmol) and 1,4-dioxane (25 mL) were added, nitrogen gas was purged, and heated to 100° C. under nitrogen gas protection for reaction. After the reaction ended, the reaction mixture was quenched by water, extracted by methylene dichloride to give a crude product. The crude product was purified by column chromatography (methylene dichloride:n-hexane=1:50 (volume ratio)), to obtain M6-B′ (1.7 g, 64% yield).

Synthesis of M6-C: In a three-necked bottle of 50 mL, M6-B (10 mmol), M6-B′ (10 mmol), sodium bicarbonate (20 mmol), tetrakis(triphenylphosphine)palladium (0.2 mmol), tetrahydrofuran (20 mL) and water (10 mL) were added, nitrogen gas was purged, and heated to 60° C. under nitrogen gas protection to react overnight. After the reaction ended, the reaction mixture was quenched by water, extracted by methylene dichloride, and the organic solvent was removed by rotary evaporation to give a crude product. The crude product was purified by column chromatography (ethyl acetate:n-hexane=1:50 (volume ratio)), to obtain M6-C (3.06 g, 92% yield).

Synthesis of M6-D: In a three-necked bottle of 50 mL, M6-C (10 mmol), (methoxymethyl)triphenylphosphonium chloride (20 mmol), tetrahydrofuran (10 mL) were added, and the temperature was reduced to 0° C. Potassium tert-butoxide (2 mmol) was resolved in 5 mL tetrahydrofuran. The three-necked bottle was purged with nitrogen gas. Under nitrogen gas protection, the potassium tert-butoxide solution was added dropwise at 0° C. to obtain a mixture. After the addition, the mixture was stirred to react for half an hour. After the reaction ended, the reaction mixture was quenched by water, extracted by methylene dichloride, and the organic solvent was removed by rotary evaporation to give a crude product. The crude product was purified by column chromatography (ethyl acetate:n-hexane=1:50 (volume ratio)), to obtain M6-D (1.8 g, 50% yield).

Synthesis of M6-E: In a three-necked bottle of 25 mL, M6-D (1 mmol) and hexafluoroisopropanol (5 mL) were added, the temperature was reduced to 0° C., and nitrogen gas was purged. Under nitrogen gas protection, trifluoromethanesulfonic acid (1 mL) was added dropwise to obtain a mixture, and the mixture was stirred to react for half an hour to give a crude product. The crude product was purified by column chromatography (ethyl acetate:n-hexane=1:50 (volume ratio)), to obtain M6-E (0.24 g, 73% yield).

Synthesis of M6-F: In a three-necked bottle of 50 mL, M6-E (10 mmol), bis(pinacolato)diboron (12 mmol), sodium acetate (20 mmol), tris(dibenzylideneacetone)dipalladium(0) (0.5 mmol) and 2-dicyclohexylphosphino-2′,6′-dimethoxybiphenyl (1.5 mmol) were added, then 1,4-dioxane (20 mL) was added, nitrogen gas was purged for three times, and heated to 100° C. under nitrogen gas protection for reaction. After the reaction ended, the reaction mixture was quenched by water, extracted by methylene dichloride, and the organic solvent was removed by rotary evaporation to give a crude product. The crude product was purified by column chromatography (ethyl acetate:n-hexane=1:50 (volume ratio)), to obtain M6-F (3.24 g, 77% yield).

Synthesis of M6: In a three-necked bottle of 100 mL, a stir bar was put at the bottom and a refluxing tube was connected on the top. The bottle was dried and purged with nitrogen gas, and M6-F (10 mmol), M6-G (10 mmol CAS1689576-03-1), sodium bicarbonate (23 mmol), tetrakis(triphenylphosphine)palladium (0.5 mmol), bis(di-tert-butyl(4-dimethylaminophenyl)phosphine) dichloropalladium(II) (0.5 mmol), toluene (25 mL), ethanol (7 mL) and water (7 mL) were separately added, nitrogen gas was purged for three times, and heated to 80° C. to react for 8 h. After the reaction ended, the reaction mixture was extracted by ethyl acetate, and the resulting extract was dried by magnesium sulfate, filtered, and dried by rotary evaporation to give a crude product. The crude product was purified by column chromatography (ethyl acetate:n-hexane=1:10 (volume ratio)), to obtain compound M6 (4.13 g, 69% yield).

Anal. Calcd. C₄₁H₂₆N₆: C, 81.71; H, 4.35; N, 13.94. Found: C, 81.78; H, 4.33; N, 13.89. HRMS (ESI) m/z [M+H]⁺: Calcd.: 602.22. Found: 603.40.

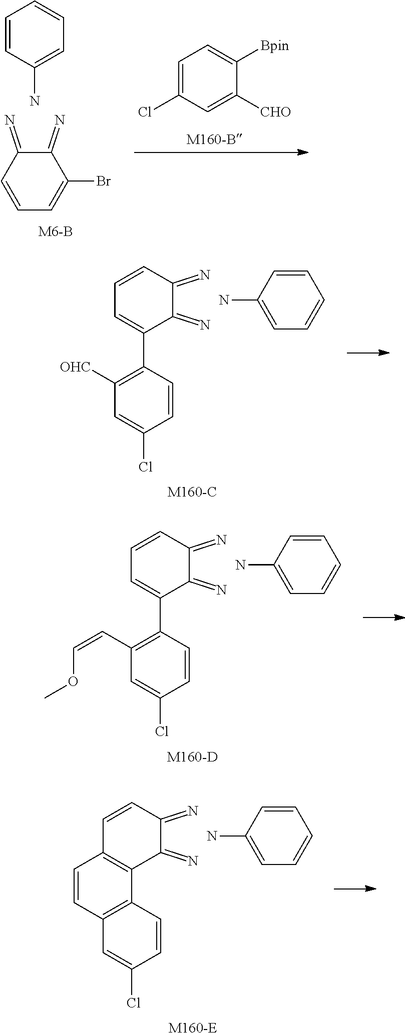

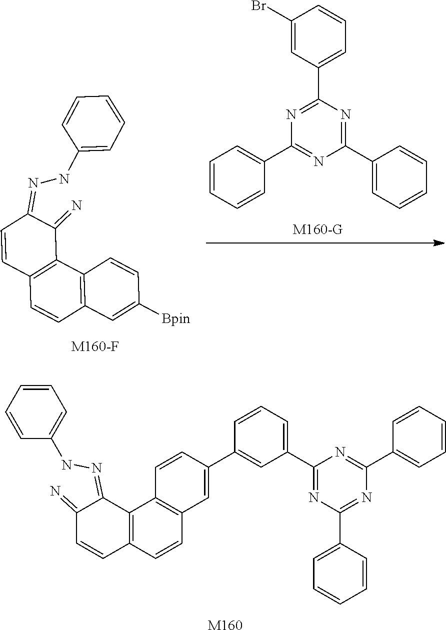

Synthesis of M160-B″: Similar to the synthesis of M6-B′, with the difference that 2-bromo-5-chlorobenzaldehyde is used to replace 2-bromo-4-chlorobenzaldehyde, to obtain M160-B″ (1.60 g, 60% yield).

Synthesis of M160-C: Similar to the synthesis of M6-C, with the difference that 4-fluoro-2-formylbenzeneboronic acid pinacol ester is used to replace 5-fluoro-2-formylbenzeneboronic acid pinacol ester, to obtain M160-C (2.13 g, 64% yield).

Synthesis of M160-D: Similar to the synthesis of M6-D, with the difference that M160-C is used to replace M6-C, to obtain M160-D (3.21 g, 89% yield).

Synthesis of M160-E: Similar to the synthesis of M6-E, with the difference that M160-D is used to replace M6-D, to obtain M160-E (0.16 g, 48% yield).

Synthesis of M160-F: Similar to the synthesis of M6-F, with the difference that M160-E is used to replace M6-E, to obtain M160-F (4.00 g, 95% yield).

Synthesis of M160: Similar to the synthesis of M6, with the difference that M160-F is used to replace M6-F, and M160-G is used to replace M6-G, to obtain M160 (4.70 g, 78% yield).

Anal. Calcd. C₄₁H₂₆N₆: C, 81.71; H, 4.35; N, 13.94. Found: C, 81.73; H, 4.37; N, 13.90. HRMS (ESI) m/z (M⁺): Calcd.: 602.22. Found: 603.29.

The corresponding products shown in Table 1 were prepared by the above-mentioned preparation method using the Material 1 and Material 2 as raw materials. The structure and characteristic data of the products are shown in Table 2.

TABLE 1

| Material 1 | Material 2 | Product | Yield (%) |
|---|---|---|---|
| 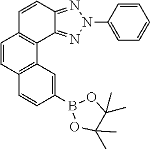 [SMILES: CC1(C)OB(c2ccc3ccc4ccc5nn(-c6ccccc6)nc5c4c3c2)OC1(C)C] | 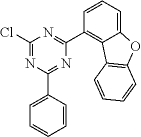 [SMILES: Clc1nc(-c2ccccc2)nc(-c2cccc3oc4ccccc4c23)n1] CAS2142681-84-1 | 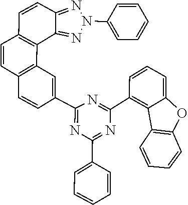 | 65 |
| | | M46 | |
| 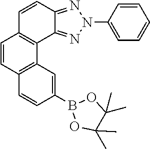 [SMILES: CC1(C)OB(c2ccc3ccc4ccc5nn(-c6ccccc6)nc5c4c3c2)OC1(C)C] | 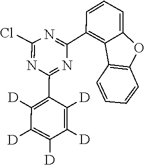 [SMILES: [2H]c1c([2H])c([2H])c(-c2nc(Cl)nc(-c3cccc4oc5ccccc5c34)n2)c([2H])c1[2H]] CAS2391956-00-4 | 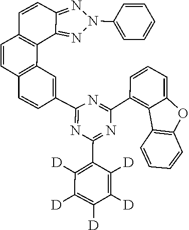 | 67 |
| | | M41 | |
| 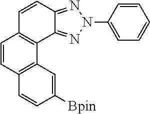 | 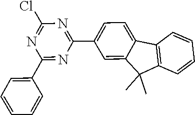 [SMILES: CC1(C)c2ccccc2-c2ccc(-c3nc(Cl)nc(-c4ccccc4)n3)cc21] CAS1618106-98-1 | 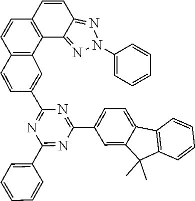 [SMILES: CC1(C)c2ccccc2-c2ccc(-c3nc(-c4ccccc4)nc(-c4ccc5ccc6ccc7nn(-c8ccccc8)nc7c6c5c4)n3)cc21] | 74 |
| | | M23 | |
| 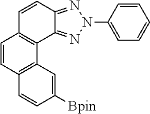 | 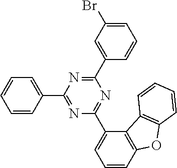 [SMILES: Brc1cccc(-c2nc(-c3ccccc3)nc(-c3cccc4oc5ccccc5c34)n2)c1] CAS2102445-28-1 | 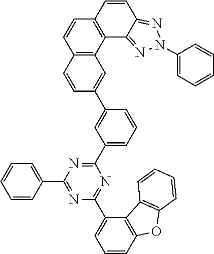 [SMILES: c1ccc(-c2nc(-c3cccc(-c4ccc5ccc6ccc7nn(-c8ccccc8)nc7c6c5c4)c3)nc(-c3cccc4oc5ccccc5c34)n2)cc1] | 65 |
| | | M97 | |
| 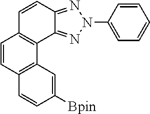 | 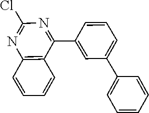 [SMILES: Clc1nc(-c2cccc(-c3ccccc3)c2)c2ccccc2n1] CAS1413365-66-8 | 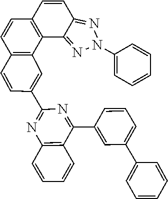 [SMILES: c1ccc(-c2cccc(-c3nc(-c4ccc5ccc6ccc7nn(-c8ccccc8)nc7c6c5c4)nc4ccccc34)c2)cc1] | 68 |
| | | M117 | |
| 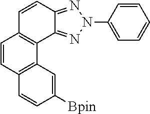 | 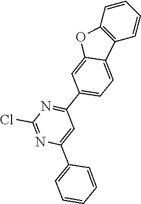 [SMILES: Clc1nc(-c2ccccc2)cc(-c2ccc3c(c2)oc2ccccc23)n1] CAS2286234-09-9 | 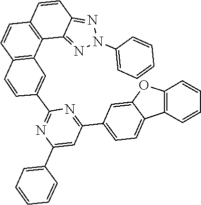 [SMILES: c1ccc(-c2cc(-c3ccc4c(c3)oc3ccccc34)nc(-c3ccc4ccc5ccc6nn(-c7ccccc7)nc6c5c4c3)n2)cc1] M93 | 68 |
| 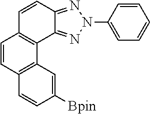 | 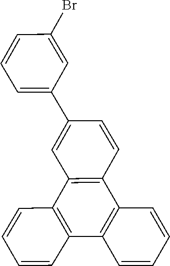 [SMILES: Brc1cccc(-c2ccc3c4ccccc4c4ccccc4c3c2)c1] | 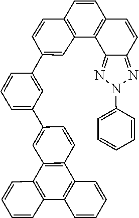 [SMILES: c1ccc(-n2nc3ccc4ccc5ccc(-c6cccc(-c7ccc8c9ccccc9c9ccccc9c8c7)c6)cc5c4c3n2)cc1] N2 | 63 |
| | CAS 1313514-53-2 | | |
| 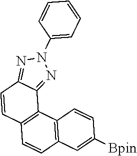 | 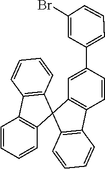 [SMILES: Brc1cccc(-c2ccc3c(c2)C2(c4ccccc4-c4ccccc42)c2ccccc2-3)c1] CAS 1556069-52-3 | 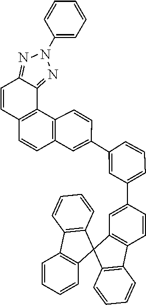 [SMILES: c1ccc(-n2nc3ccc4ccc5cc(-c6cccc(-c7ccc8c(c7)C7(c9ccccc9-c9ccccc97)c7ccccc7-8)c6)ccc5c4c3n2)cc1] | 63 |
| | | N76 | |
| 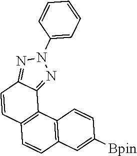 | 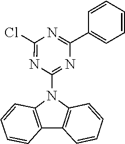 [SMILES: Clc1nc(-c2ccccc2)nc(-n2c3ccccc3c3ccccc32)n1] CAS 1268244-56-9 | 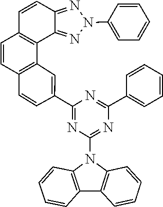 [SMILES: c1ccc(-c2nc(-c3ccc4ccc5ccc6nn(-c7ccccc7)nc6c5c4c3)nc(-n3c4ccccc4c4ccccc43)n2)cc1] | 61 |
| | | M31 | |

TABLE 2

| | | | HRMS (ESI) | |
|---|---|---|---|---|
| Com- | Elemental analysis | | m/z [M + H]⁺ | |
| pound | Calcd. | Found | Calcd. | Found |
| M46 | C, 79.85; H, 3.92; N, | C, 79.92; H, 3.91; N, | 616.20 | 617.26 |
| | 13.63; | 13.59; | | |
| M41 | C, 79.21; H, 4.70; N, | C, 79.24; H, 4.71; N, | 621.23 | 622.26 |
| | 13.52; | 13.48; | | |
| M23 | C, 82.22; H, 4.70; N, | C, 82.16; H, 4.72; N, | 642.25 | 643.25 |
| | 13.08; | 13.12; | | |
| M97 | C, 81.49; H, 4.07; N, | C, 81.53; H, 4.08; N, | 692.23 | 693.20 |
| | 12.13; | 12.08; | | |
| M117 | C, 83.46; H, 4.38; N, | C, 83.39; H, 4.40; N, | 575.21 | 576.24 |
| | 12.17; | 12.21; | | |
| M93 | C, 81.93; H, 4.09; N, | C, 81.89; H, 4.11; N, | 615.21 | 616.17 |
| | 11.37; | 11.40; | | |
| N2 | C, 88.42; H, 4.55; N, | C, 88.47; H, 4.53; N, | 597.22 | 598.20 |
| | 7.03; | 7.00; | | |
| N76 | C, 89.32; H, 4.56; N, | C, 89.28; H, 4.57; N, | 685.25 | 686.28 |
| | 6.13; | 6.15; | | |
| M31 | C, 79.98; H, 4.09; N, | C, 80.04; H, 4.08; N, | 615.22 | 616.16 |
| | 15.92; | 15.88; | | |

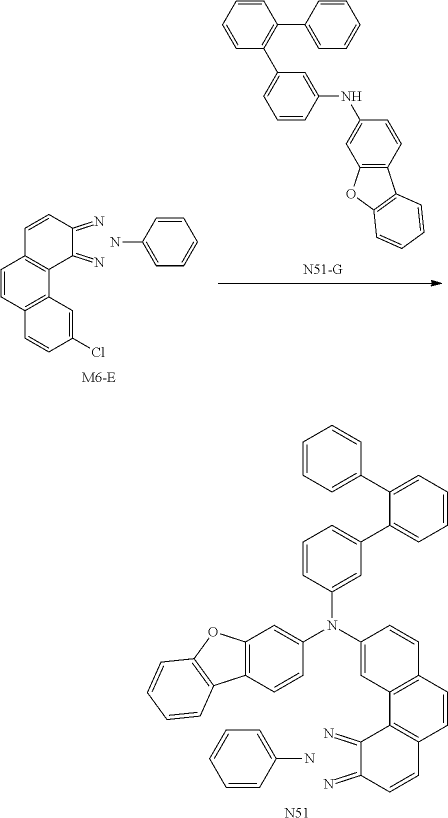

Synthesis of compound N51: In a three-necked bottle of 25 mL, nitrogen gas was purged, and then M6-E (1 mmol), compound N51-G (1 mmol), sodium tert-butoxide (2 mmol), tris(dibenzylideneacetone)dipalladium(0) (0.02 mmol), 50% tri-tert-butylphosphine solution (0.1 mmol) and toluene 8 mL was added, and stirred under reflux for reaction. After the reaction ended, the reaction mixture was cooled to room temperature, and the organic layer was extracted by ethyl acetate and H₂O, and the extracted organic layer was dried by magnesium sulfate, filtered, and the filtrate was concentrated under vacuum to give a crude product. The crude product was purified by column chromatography (ethyl acetate:n-hexane=1:50 (volume ratio)), to obtain compound N51 (0.50 g, 71% yield).

Anal. Calcd. C₅₀H₃₂N₄O: C, 85.20; H, 4.58; N, 7.95. Found: C, 85.21; H, 4.60; N, 7.92. HRMS (ESI) m/z [M+H]⁺: Calcd.: 704.26. Found: 705.31.

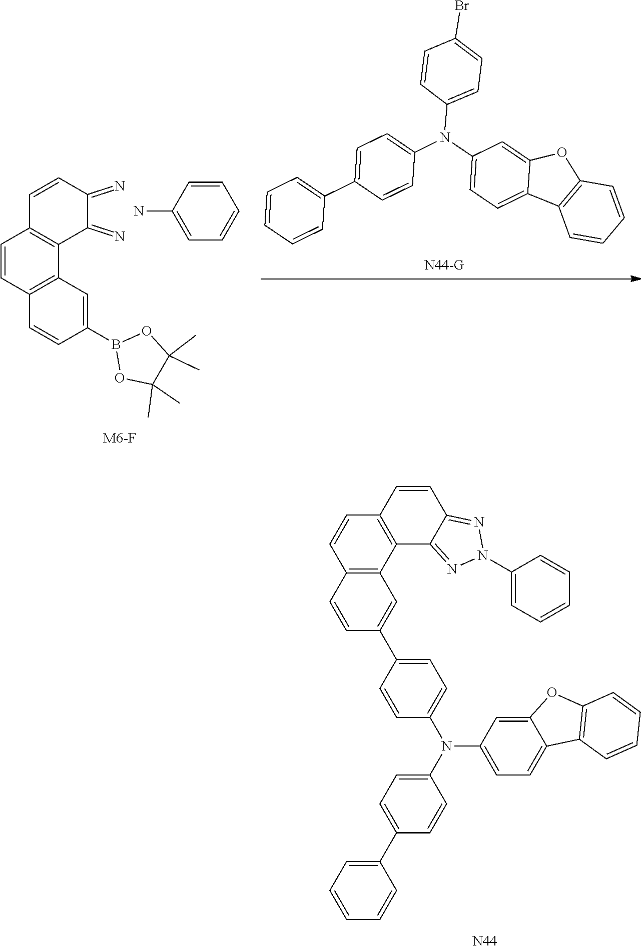

Synthesis of compound N44: In a two-necked bottle of 25 mL, a stir bar was put at the bottom and a refluxing tube was connected on the top. The bottle was dried and purged with nitrogen gas, and M6-F (0.01 mol), N44-G (0.01 mol), potassium carbonate (0.013 mol), tetrakis(triphenylphosphine)palladium (0.5 mmol), toluene (10 mL) and water (4 mL) was separately added, nitrogen gas was purged for three times, and heated to 85° C. under nitrogen gas protection to react for 10 h. After the reaction ended, the reaction mixture was extracted by ethyl acetate, and the extracted solution was dried by magnesium sulfate, filtered, and dried by rotary evaporation to give a crude product. The crude product was purified by column chromatography (ethyl acetate:n-hexane=1:10 (volume ratio)), to obtain compound N44 (4.44 g, 63% yield).

Anal. Calcd. C₅₀H₃₂N₄O: C, 85.20; H, 4.58; N, 7.95. Found: C, 85.16; H, 4.60; N, 7.98. HRMS (ESI) m/z (M⁺): Calcd.: 704.26. Found: 705.28.

The corresponding products shown in Table 3 were prepared by the above-mentioned preparation method using the Material 1 and Material 2 as raw materials. The structure and characteristic data of the products are shown in Table 4.

TABLE 3

| Material 1 | Material 2 | Product | Yield % |
|---|---|---|---|
| 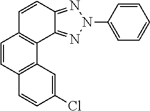 [SMILES: Clc1ccc2ccc3ccc4nn(-c5ccccc5)nc4c3c2c1] | 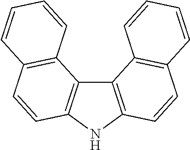 [SMILES: c1ccc2c(c1)ccc1[nH]c3ccc4ccccc4c3c12] CAS 194-59-2 | 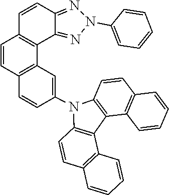 | 62 |
| | | N12 | |
| 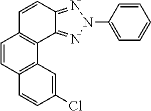 [SMILES: Clc1ccc2ccc3ccc4nn(-c5ccccc5)nc4c3c2c1] | 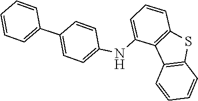 [SMILES: c1ccc(-c2ccc(Nc3cccc4sc5ccccc5c34)cc2)cc1] CAS 1429933-62-9 | 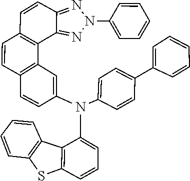 | 60 |
| | | N60 | |
| 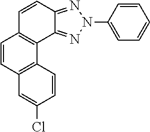 [SMILES: Clc1ccc2c(ccc3ccc4nn(-c5ccccc5)nc4c32)c1] | 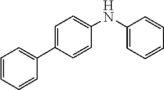 [SMILES: c1ccc(Nc2ccc(-c3ccccc3)cc2)cc1] CAS32228-99-2 | 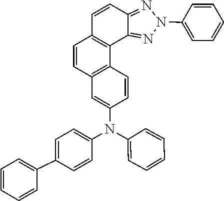 | 61 |
| | | N83 | |
| 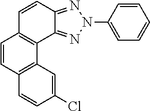 [SMILES: Clc1ccc2ccc3ccc4nn(-c5ccccc5)nc4c3c2c1] | 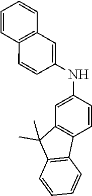 [SMILES: CC1(C)c2ccccc2-c2ccc(Nc3ccc4ccccc4c3)cc21] | 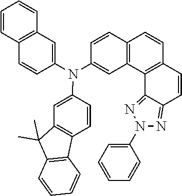 | 64 |
| | CAS 1263001-82-6 | N40 | |

TABLE 4

| | | | HRMS (ESI) | |
|---|---|---|---|---|
| Com- | Elemental analysis | | m/z [M + H]⁺ | |
| pound | Calcd. | Found | Calcd. | Found |
| N12 | C, 85.69; H, 4.31; N, | C, 85.75; H, 4.29; | 560.20 | 561.11 |
| | 9.99 | N, 9.96 | | |
| N60 | C, 81.96; H, 4.38; N, | C, 81.91; H, 4.40; | 644.20 | 645.30 |
| | 8.69; S, 4.97 | N, 8.71; S, 4.98 | | |
| N83 | C, 84.73; H, 4.87; N, | C, 84.70; H, 4.85; | 538.22 | 539.31 |
| | 10.40 | N, 10.45 | | |
| N40 | C, 85.96; H, 5.13; N, | C, 86.01; H, 5.10; | 628.26 | 629.33 |
| | 8.91 | N, 8.89 | | |

### Element Examples 1 to 16 and Comparative Element Examples 1 to 2

An organic electroluminescence element having the following layer structure was provided: base (indium tin oxide (ITO) coated glass substrate, as an anode)/hole injection layer (HIL)/hole transport layer (HTL)/emitting layer (EML)/electron transport layer (ETL)/electron injection layer (EIL), and the cathode at last.

The materials needed to prepare OLEDs are listed below, wherein the REF-1 is comparative compound 1:

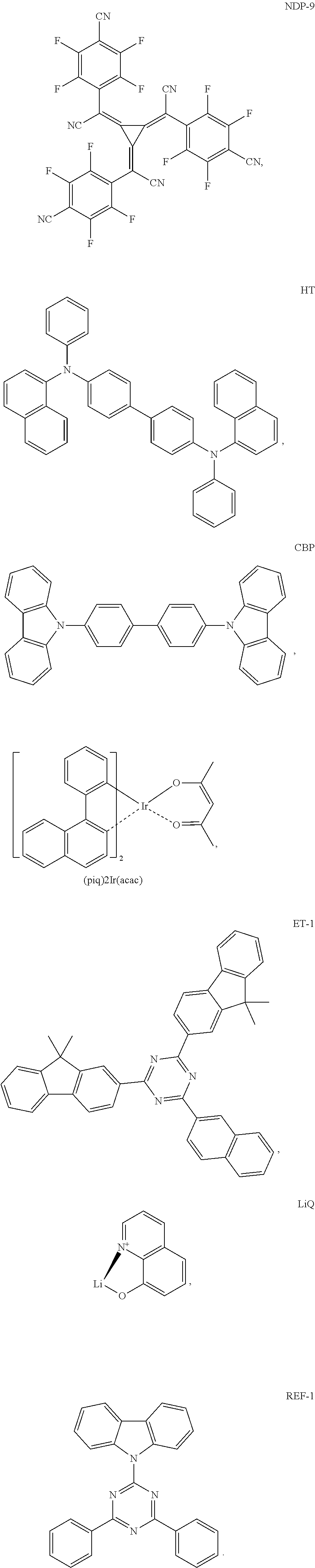

The above-mentioned organic electroluminescence element was prepared by the following steps:

(1) Cleaning the substrate: a glass substrate coated with transparent ITO (the anode layer) was ultrasonicated in an aqueous detergent (the content and concentration of the aqueous detergent: an ethylene glycol solvent of ≤10 percent by weight (wt %), triethanolamine of ≤1 wt %), washed in deionized water, degreased in an acetone/ethanol mixed solvent (volume ratio=1:1) by ultrasonication, baked in a clear environment until water was completely removed, and washed by ozone under ultraviolet light;

(2) Depositing emitting functional layers:

The glass substrate with the anode layer was placed in a chamber, and the chamber was vacuumized until 1×10⁻⁶ Pascal (Pa) to 2×10⁻⁴ Pa, and a mixture of NDP-9 and HT (mass ratio of NDP-9 and HT is 3:97) was deposited on the anode layer in vacuum to form a hole injection layer, in which the deposited thickness was 10 nanometers (nm).

A hole transport layer (material: HT) was deposited on the hole injection layer, in which the deposited thickness was 80 nm.

An emitting layer was deposited on the hole transport layer. Specifically, the preparation method was: the light-emitting host material (materials shown in Table 5) and a guest material (piq)₂Ir(acac) were co-deposited in vacuum, in which the total deposited thickness was 30 nm.

An electron transport layer was deposited on the emitting layer. Specifically, the preparation method was: the electron transport layer materials (shown in Table 5) were co-deposited in vacuum, in which the total deposited thickness was 30 nm.

An electron injection layer (material: LiQ) was deposited on the electron transport layer, in which the total deposited thickness was 1 nm.

Al (cathode) was deposited on the electron injection layer, in which the deposited thickness was 80 nm.

The materials (mat.) of each layer in the element and parameters such as thickness (thk.) of Element Examples 1 to 16 (E1 to E16) and Comparative Element Examples 1 to 2 (CE1 to CE2) are shown in Table 5.

TABLE 5

| | HIL mat./ | HTL mat./ | EML mat./ | ETL mat./ | EIL mat./ | Cathode mat./ |
|---|---|---|---|---|---|---|
| No. | thk. | thk. | thk. | thk. | thk. | thk. |
| E1 | NDP-9:HT | HT/ | CBP:(piq)₂Ir(acac) | compoundM6:LiQ/ | LiQ/ | Al/ |
| | (mass ratio | 80 nm | (mass ratio | (mass ratio | 1 nm | 80 nm |
| | 3:97)/10 nm | | 95:5)/30 nm | 1:1)/30 nm | | |
| E2 | NDP-9:HT | HT/ | compound M6:compound | ET-1:LiQ | LiQ/ | Al/ |
| | (mass ratio | 80 nm | N12:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E3 | NDP-9:HT | HT/ | compound M46:compound | ET-1:LiQ | LiQ/ | Al/ |
| | (masratio | 80 nm | N40:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E4 | NDP-9:HT | HT/ | compound M41:compound | ET-1:LiQ | LiQ/ | Al/ |
| | (mass ratio | 80 nm | N40:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E5 | NDP-9:HT | HT/ | compound M23:compound | ET-1:LiQ | LiQ/ | Al/ |
| | (mass ratio | 80 nm | N60:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E6 | NDP-9:HT | HT/ | compound M97:compound | ET-1:LiQ | LiQ/ | Al/ |
| | (mass ratio | 80 nm | N51:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E7 | NDP-9:HT | HT/ | compound M97:compound | ET-1:LiQ | LiQ/ | Al/ |
| | (mass ratio | 80 nm | N76:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E8 | NDP-9:HT | HT/ | compound M117:compound | ET-1:LiQ | LiQ/ | Al/ |
| | (mass ratio | 80 nm | N2:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E9 | NDP-9:HT | HT/ | compound M93:compound | ET-1:LiQ | LiQ/ | Al/ |
| | (mass ratio | 80 nm | N60:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E10 | NDP-9:HT | HT/ | compound M160:compound | ET-1:LiQ | LiQ/ | Al/ |
| | (mass ratio | 80 nm | N40:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E11 | NDP-9:HT | HT/ | compound M31:compound | ET-1:LiQ | LiQ/ | Al/ |
| | (mass ratio | 80 nm | N60:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E12 | NDP-9:HT | HT/ | compound M97:compound | ET-1:LiQ | LiQ/ | Al/ |
| | (mass ratio | 80 nm | N44:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E13 | NDP-9:HT | HT/ | compound M117:compound | ET-1:LiQ | LiQ/ | Al/ |
| | (mass ratio | 80 nm | N83:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E14 | NDP-9:HT | HT/ | M117:(piq)₂Ir(acac) | ET-1:LiQ | LiQ/ | Al/ |
| | (mass ratio | 80 nm | (mass ratio | (mass ratio | 1 nm | 80 nm |
| | 3:97)/10 nm | | 95:5)/30 nm | 1:1)/30 nm | | |
| E15 | NDP-9:HT | HT/ | compound | ET-1:LiQ | LiQ/ | Al/ |
| | (mass ratio | 80 nm | M6:CBP:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| E16 | NDP-9:HT | HT/ | compound | ET-1:LiQ | LiQ/ | Al/ |
| | (mass ratio | 80 nm | N51:CBP:(piq)₂Ir(acac) | (mass ratio | 1 nm | 80 nm |
| | 3:97)/10 nm | | (mass ratio | 1:1)/30 nm | | |
| | | | 47.5:47.5:5)/30 nm | | | |
| CE1 | NDP-9:HT | HT/ | CBP:(piq)₂Ir(acac) | ET-1:LiQ | LiQ/ | Al/ |
| | (mass ratio | 80 nm | (mass ratio | (mass ratio | 1 nm | 80 nm |
| | 3:97)/10 nm | | 95:5)/30 nm | 1:1)/30 nm | | |
| CE2 | NDP-9:HT | HT/ | REF-1:CBP:(piq)₂Ir(acac) | ET-1:LiQ | LiQ/ | Al/ |
| | (mass ratio | 80 nm | (mass ratio | (mass ratio | 1 nm | 80 nm |
| | 3:97)/10 nm | | 47.5:47.5:5)/30 nm | 1:1)/30 nm | | |

Characteristic Tests:

Instruments: the characteristics such as current, voltage, luminance and the like of the elements of the above Element Examples 1 to 16 and Comparative Element Examples 1 to 2 were synchronously tested by PR 650 SpectraScan Colorimeter and Keithley K 2400 SourceMeter;

Testing conditions of Element Examples 1 to 16 and Comparative Element Examples 1 to 2:

Conditions for testing electrooptical characteristics: a current density of 10 milliamperes/square centimeter (mA/cm²) under room temperature;

Service life test: tested with a current density of 50 mA/cm² under room temperature, and the time period recorded when the luminance of the tested element was reduced to 98% of the original luminance (in hour).

The test results of the elements are shown in Table 6.

TABLE 6

| | Driving | Current efficiency | Service |
|---|---|---|---|
| No. | voltage (V) | (Cd/A) | life (h) |
| E1 | 4.4 | 12 | 21 |
| E2 | 3.8 | 21 | 66 |
| E3 | 3.8 | 20 | 75 |
| E4 | 3.8 | 22 | 70 |
| E5 | 3.9 | 21 | 65 |
| E6 | 3.9 | 20 | 69 |
| E7 | 3.9 | 21 | 67 |
| E8 | 3.8 | 21 | 62 |
| E9 | 3.9 | 20 | 64 |
| E10 | 4.0 | 18 | 50 |
| E11 | 3.9 | 20 | 60 |
| E12 | 3.9 | 19 | 54 |
| E13 | 4.0 | 18 | 56 |
| E14 | 4.1 | 17 | 30 |
| E15 | 4.1 | 16 | 35 |
| E16 | 4.2 | 16 | 33 |
| CE1 | 4.8 | 5 | 5 |
| CE2 | 4.6 | 8 | 12 |

From Table 6, it is clear that the compounds developed by the present invention obviously increase the charge carrier injection efficiency, reduce the energy level difference between layers, balance the electron- and hole-transport efficiency, thereby effectively increase the efficiency of the organic electroluminescence device, and prolong the service life of the organic electroluminescence device. When the organic electroluminescence material is used as the material of the organic functional layer, it makes the element have a lower driving voltage (4.4 voltages (V) or lower, especially to 4.0 V or lower), a higher current efficiency (12 Candelas/Ampere (Cd/A) or more, especially to 18 Cd/A or more) and a longer service life (15 h or more, especially to 50 h or more).

The applicant claims herein that even though the organic electroluminescence material of the present invention and the organic electroluminescence device and electronic products comprising organic electroluminescence material are demonstrated by the above examples, the scope of the present invention is not limited by these examples. That is to say, it does not mean that the present invention has to be carried out based on the above examples. Those skilled in the art should understand that any improvement of the present invention, equivalent replacement of materials, addition of auxiliary components, selection of specific means and the like are all within the scope of protection and disclosure of the present invention.

What is claimed is:

1. A compound, characterized in that the compound has a structure represented by Formula (1): 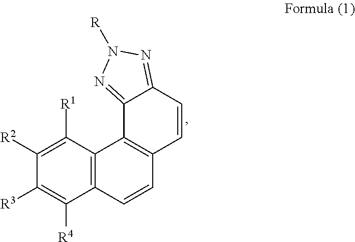
wherein, R is selected from hydrogen, deuterium, halogen, a cyano group, a substituted or unsubstituted C1-C30 alkyl group, a substituted or unsubstituted C3-C30 cycloalkyl group, a substituted or unsubstituted C6-C30 aryl group, and a substituted or unsubstituted C3-C30 heteroaryl group;
R¹ is -L¹Ar¹; R² is -L²Ar²; R³ is -L³Ar³; R⁴ is -L⁴Ar⁴;
L¹ to L⁴ are each independently selected from a bond, a substituted or unsubstituted C6-C30 arylene group, and a substituted or unsubstituted C3-C30 heteroarylene group; and
Ar¹ to Ar⁴ are each independently selected from hydrogen, deuterium, halogen, a cyano group, a substituted or unsubstituted C6-C60 arylamino group, a substituted or unsubstituted C3-C60 heteroarylamino group, a substituted or unsubstituted C6-C60 aryl group, and a substituted or unsubstituted C3-C60 heteroaryl group.

2. The compound as claimed in claim 1, characterized in that Ar¹ to Ar⁴ are each independently selected from hydrogen, deuterium, halogen, and a group selected from a phenyl group, a naphthyl group, a biphenylyl group, a phenanthryl group, a fluoranthenyl group, a benzophenanthryl group, a terphenylyl group, a triphenylenylene group, a dimethylfluorenyl group, a diphenylfluorenyl group, a spiro-bifluorenyl group, a benzodimethylfluorenyl group, a benzodiphenylfluorenyl group, a benzo-spiro-bifluorenyl group, a dibenzofuryl group, a benzonaphthofuryl group and a benzonaphthothiophenyl group, and a dibenzothiophenyl group, each of which is substituted or unsubstituted.

3. The compound as claimed in claim 1, characterized in that at least one of Ar¹ to Ar⁴ is selected from a group represented by Formula a: 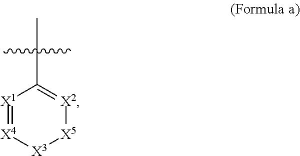
X¹ is selected from N and CR^{X1}; X² is selected from N and CR^{X2}; X³ is selected from N and CR^{X3}; X⁴ is selected from N and CR^{X4}; X⁵ is selected from N and CR^{X5};
R^{X1} to R^{X5} are each independently selected from hydrogen, deuterium, halogen, a cyano group, a substituted or unsubstituted C1-C30 alkyl group, a substituted or unsubstituted C3-C30 cycloalkyl group, a substituted or unsubstituted C6-C30 aryl group, a substituted or unsubstituted C3-C30 heteroaryl group; R^{X1} to R^{X5} are present individually without forming a ring, or any adjacent two of R^{X1} to R^{X5} joined to form a ring A, and the ring A is a substituted or unsubstituted C6-C30 aromatic ring, or a substituted or unsubstituted C3-C30 heteroaromatic ring.

4. The compound as claimed in claim 3, characterized in that the ring A is a benzene ring, a naphthalene ring, an anthracene ring, a phenanthrene ring, a benzothiophene ring, a benzofuran ring, or an indene ring;
R^{X1} to R^{X5} are each independently selected from hydrogen, deuterium, halogen, and a group selected from a phenyl group, a biphenylyl group, a terphenylyl group, a naphthyl group, a phenanthryl group, an anthryl group, a phenylnaphthyl group, a naphthylphenyl group, a pyridyl group, a bipyridyl group, a dibenzofuryl group, a dibenzothiophenyl group, a carbazolyl group, a carbazolylphenyl group, a phenylcarbazolyl group, a dimethylfluorenyl group, a diphenylfluorenyl group, a spiro-bifluorenyl group, a dibenzofurylphenyl group, a dibenzothiophenylphenyl group, a dimethylfluorenylphenyl group, a benzocarbazolyl group, a benzonaphthofuryl group and a benzonaphthothiophenyl group, each of which is substituted or unsubstituted; and
in X¹ to X⁵,
X¹ is N; X² is N; X³ is CR^{X3}; X⁴ is CR^{X4}; and X⁵ is CR^{X5}; or
X¹ is N; X³ is N; X² is CR^{X2}; X⁴ is CR^{X4}; and X⁵ is CR^{X5}; or
X¹ is N; X² is N; X³ is N; X⁴ is CR^{X4}; and X⁵ is CR^{X5}.

5. The compound as claimed in claim 1, characterized in that at least one of Ar¹ to Ar⁴ is selected from a group represented by Formula b: 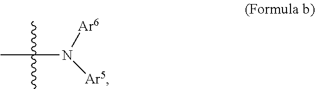
Ar⁵ to Ar⁶ are each independently selected from a substituted or unsubstituted C6-C30 aryl group, a substituted and unsubstituted C3-C30 heteroaryl group.

6. The compound as claimed in claim 5, characterized in that Ar⁵ to Ar⁶ are each independently selected from a phenyl group, a biphenylyl group, a terphenylyl group, a naphthyl group, a phenanthryl group, an anthryl group, a triphenylenylene group, a phenylnaphthyl group, a naphthylphenyl group, a pyridyl group, a bipyridyl group, a dibenzofuryl group, a dibenzothiophenyl group, a benzonaphthofuryl group, a benzonaphthothiophenyl group, a dinaphthofuryl group, a dinaphthothiophenyl group, a dibenzofurylphenyl group, a dibenzothiophenylphenyl group, a dimethylfluorenyl group, a benzodimethylfluorenyl group, a diphenylfluorenyl group, a spiro-bifluorenyl group, and a dimethylfluorenylphenyl group.

7. The compound as claimed in claim 1, characterized in that at least one of Ar¹ to Ar⁴ is selected from a group represented by Formula c: 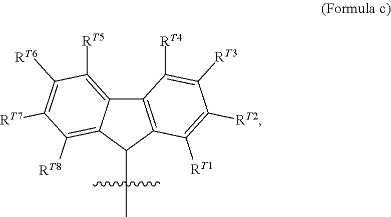
R^{T1} to R^{T8} are each independently selected from hydrogen, deuterium, halogen, a cyano group, a substituted or unsubstituted C1-C30 alkyl group, a C1-C30 alkyl group in which one or more methylene groups are independently substituted by —O— and/or —S— in a manner that O atom and/or S atom are not adjacent to each other, a substituted or unsubstituted C7-C30 arylalkyl group, a substituted or unsubstituted C6-C30 aryl group, a substituted or unsubstituted C3-C30 heteroaryl group, a substituted or unsubstituted C4-C30 heteroarylalkyl group, a substituted or unsubstituted C3-C30 cycloalkyl group, a substituted or unsubstituted C3-C30 heterocycloalkyl group, a substituted or unsubstituted C3-C30 cycloalkenyl group, a substituted or unsubstituted C1-C30 alkoxy group, and a substituted or unsubstituted C6-C30 aryloxy group;
R^{T1} to R^{T8} are present individually without forming a ring, or any adjacent two of R^{T1} to R^{T8} joined to form a ring B, and the ring B is a substituted or unsubstituted C6-C30 aromatic ring, or a substituted or unsubstituted C6-C30 heteroaromatic ring.

8. The compound as claimed in claim 7, characterized in that the Formula c is selected from any of the c-1, c-2, c-3, c-4, c-5 and c-6: 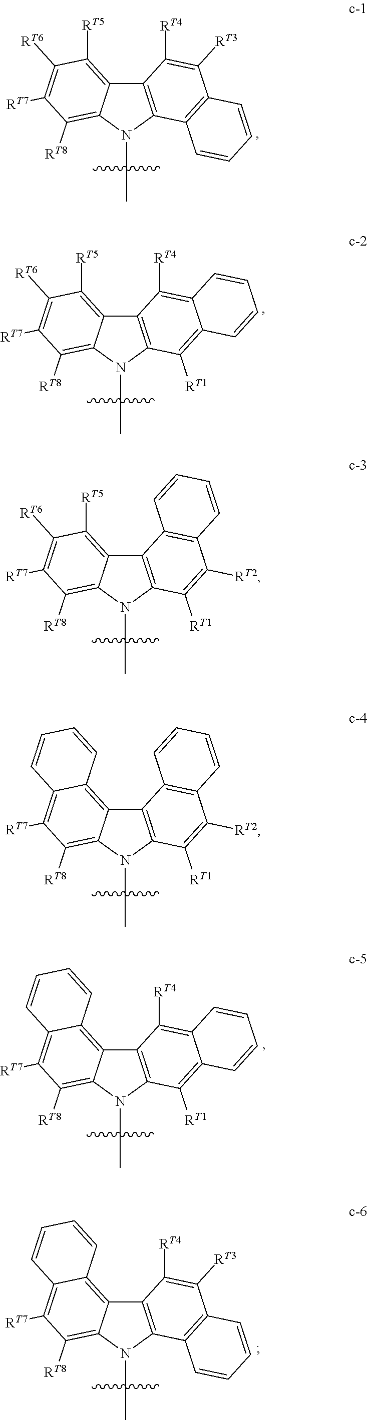
R^{T1} to R^{T8} are each independently selected from hydrogen, deuterium, and a group selected from a methyl group, an ethyl group, a tert-butyl group, an adamantyl group, a phenyl group, a biphenylyl group, a terphenylyl group, a naphthyl group, a phenanthryl group, an anthryl group, a triphenylenylene group, a phenylnaphthyl group, a naphthylphenyl group, a pyridyl group, a bipyridyl group, a dibenzofuryl group, a dibenzothiophenyl group, a benzonaphthofuryl group, a benzonaphthothiophenyl group, a dinaphthofuryl group, a dinaphthothiophenyl group, a dibenzofurylphenyl group, a dibenzothiophenylphenyl group, a dimethylfluorenyl group, a benzodimethylfluorenyl group, a diphenylfluorenyl group, a spiro-bifluorenyl group and a dimethylfluorenylphenyl group, each of which is substituted or unsubstituted.

9. The compound as claimed in claim 1, characterized in that L¹ to L⁴ are each independently selected from a bond, a substituted or unsubstituted phenylene group, a substituted or unsubstituted biphenylene group, a substituted or unsubstituted terphenylene group, and a substituted or unsubstituted naphthylene group.

10. The compound as claimed in claim 1, characterized in that the compound is a compound M having electron transport properties or a compound N having hole transport properties;
the compound M is any one of compounds M1 to M206: 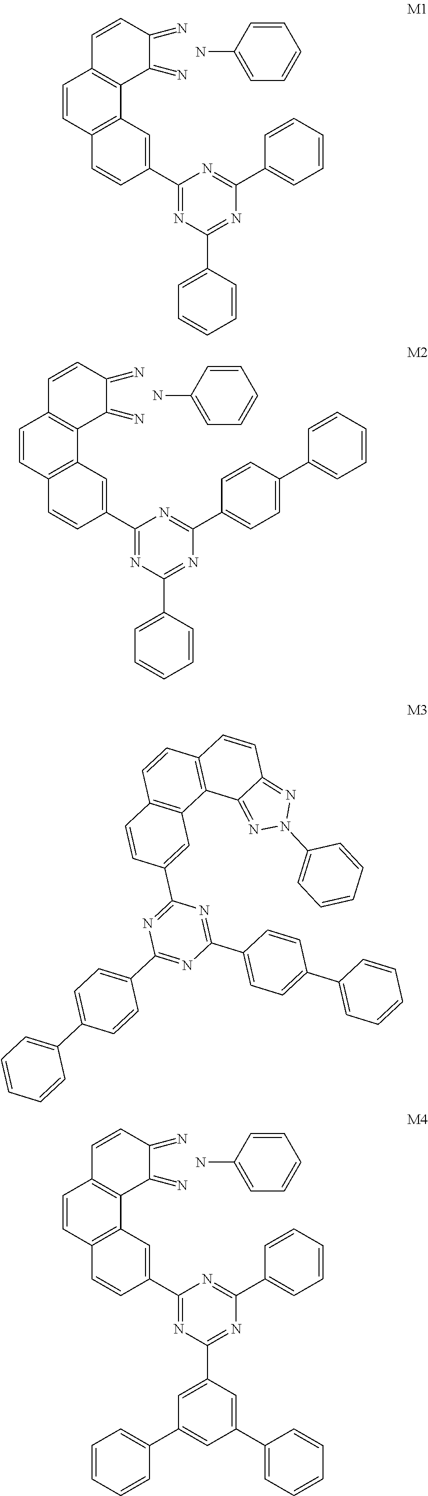 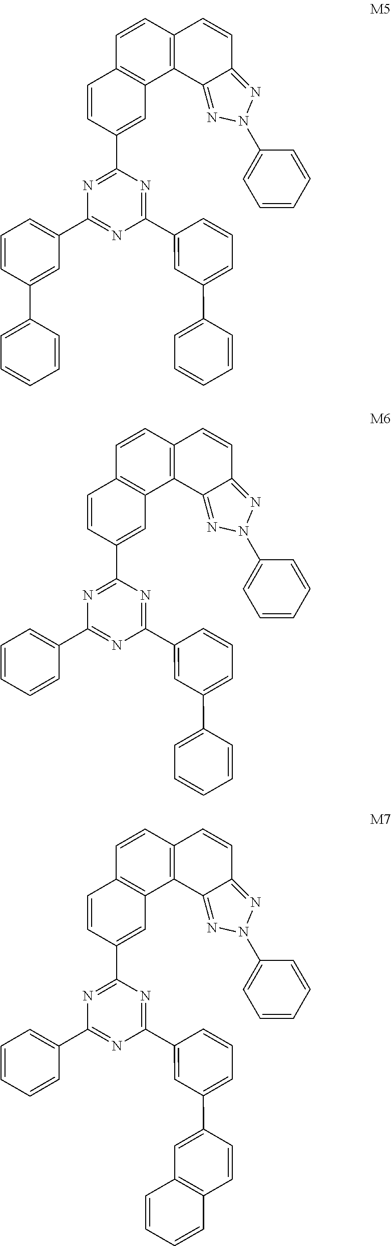 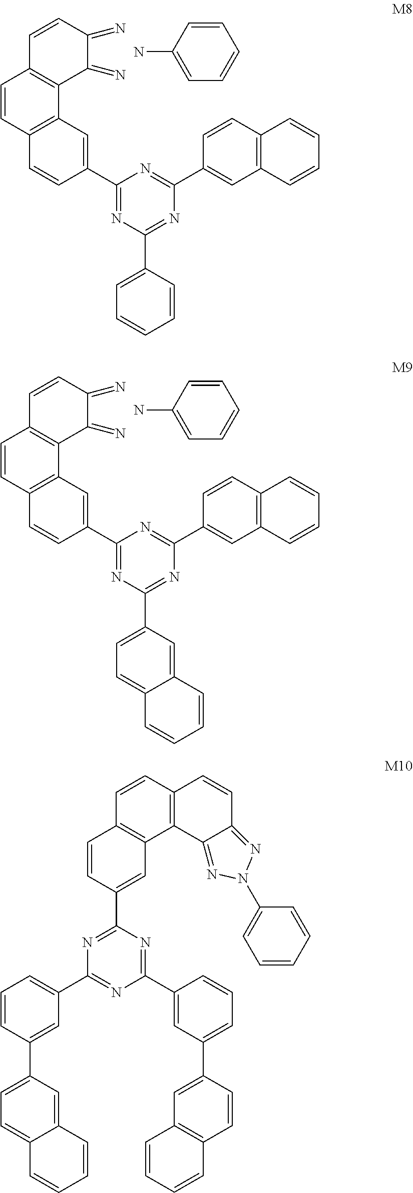 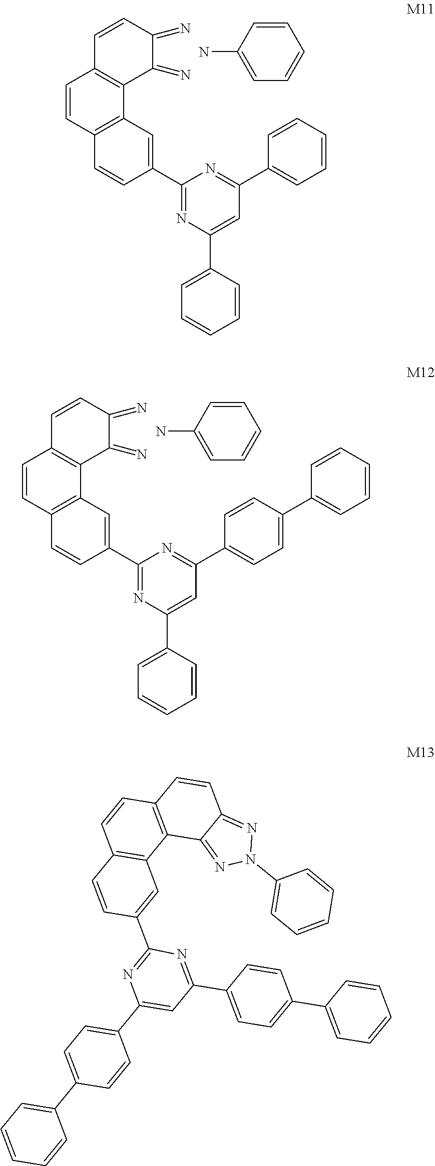 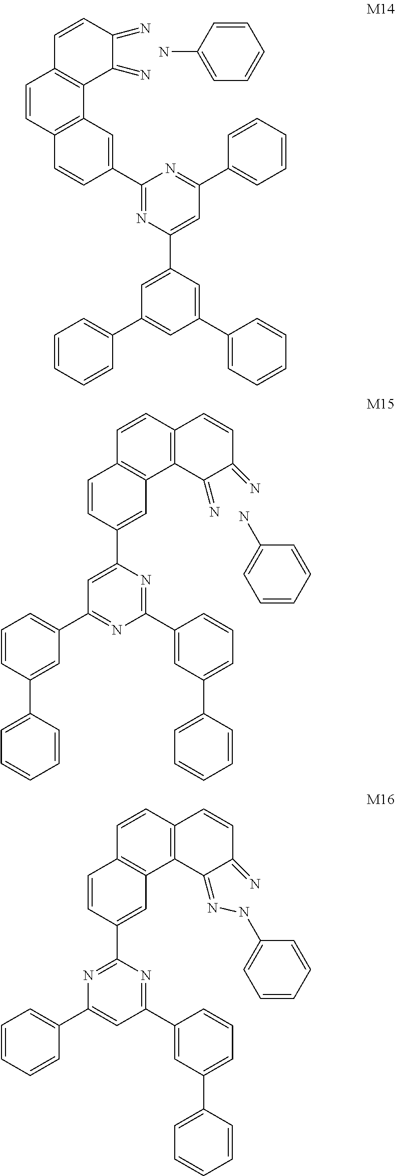 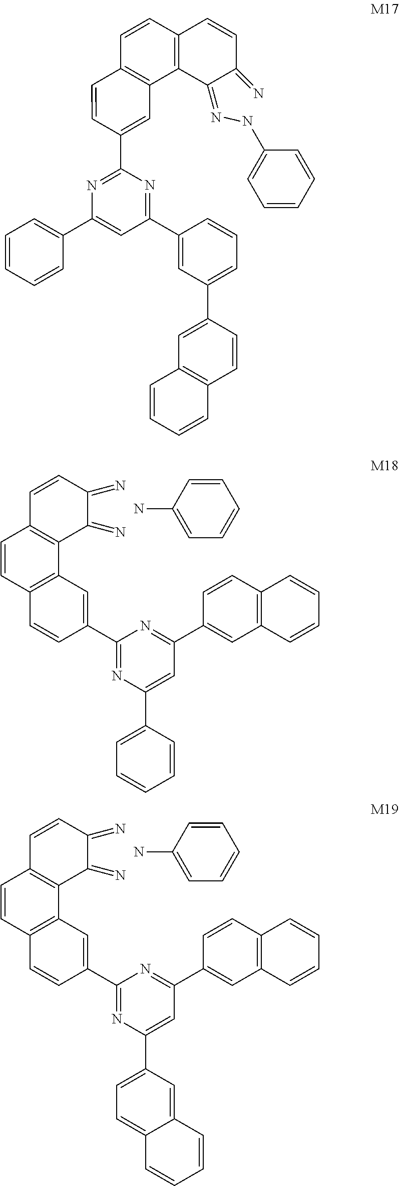 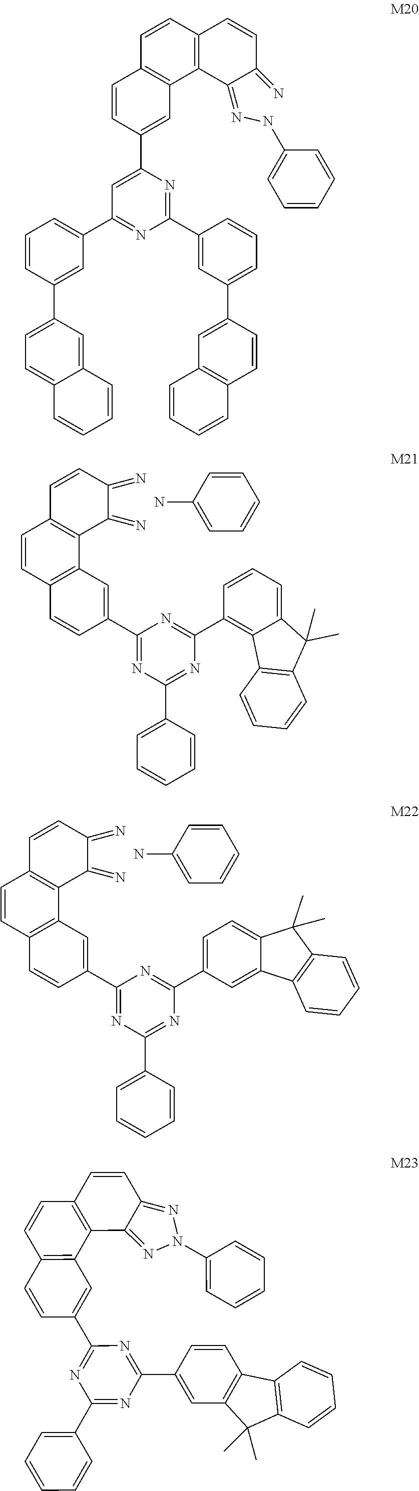 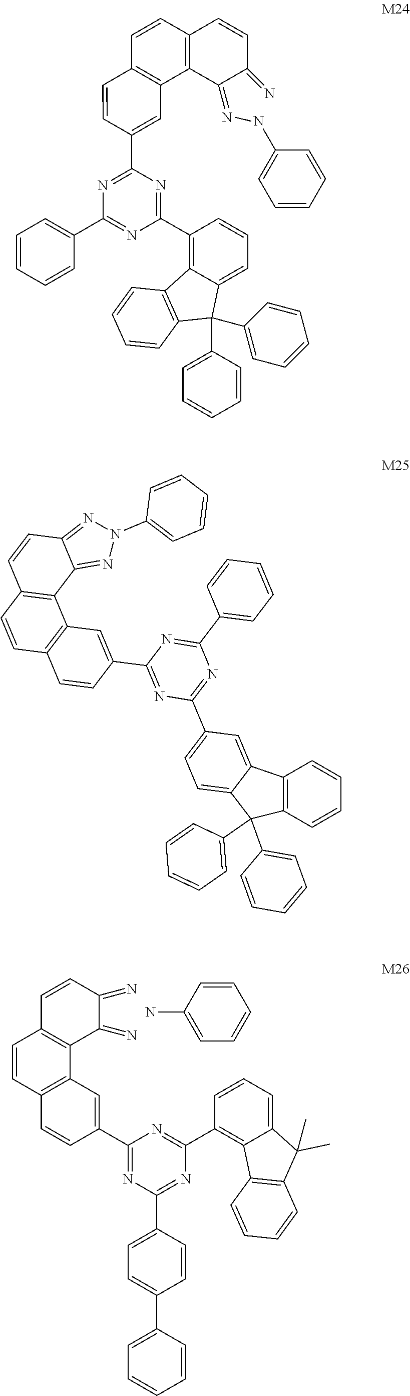 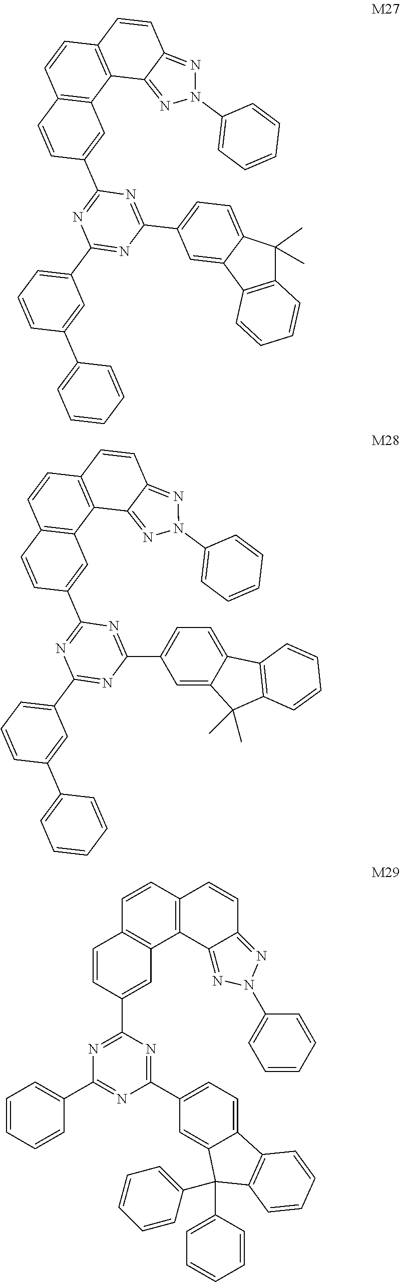 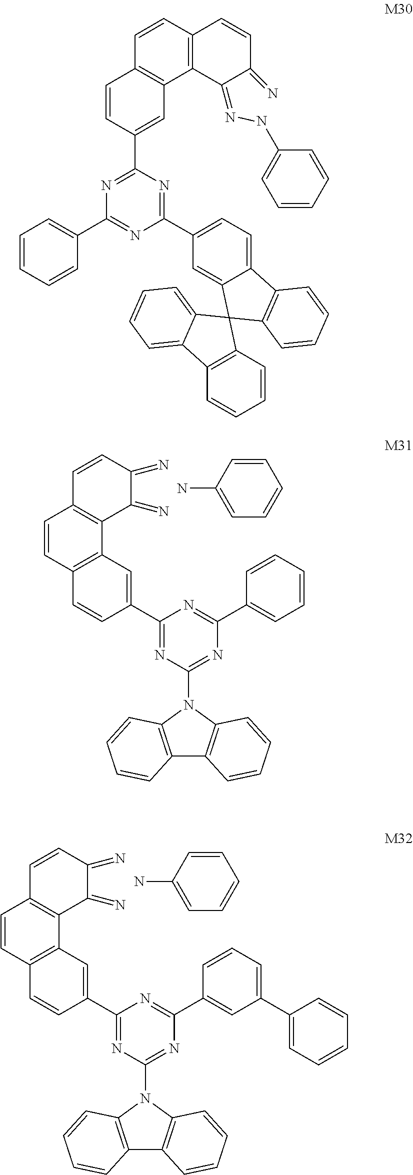 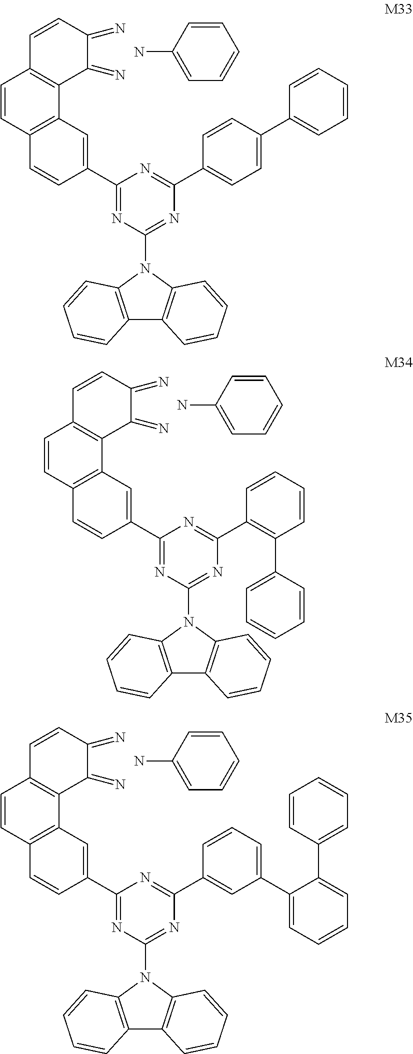 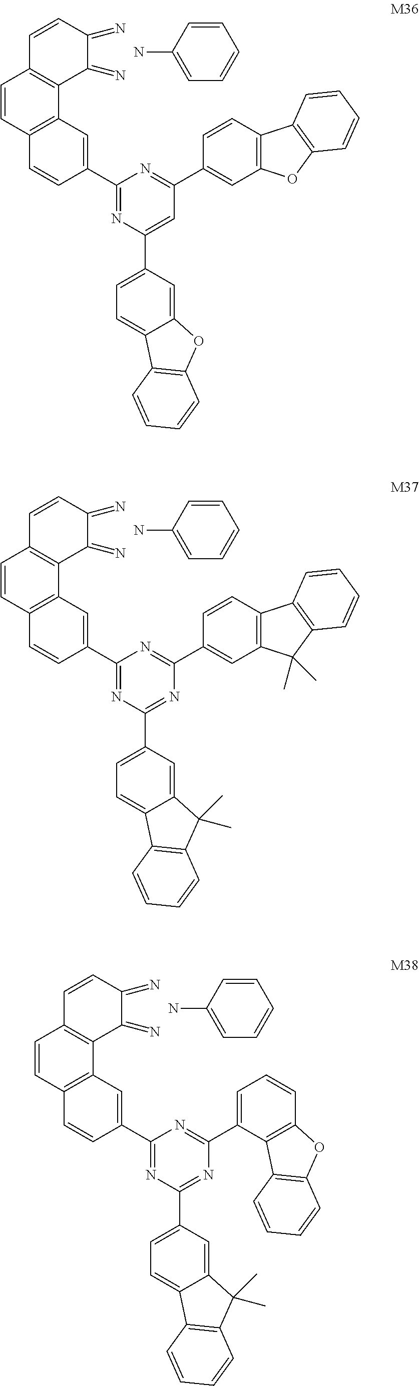 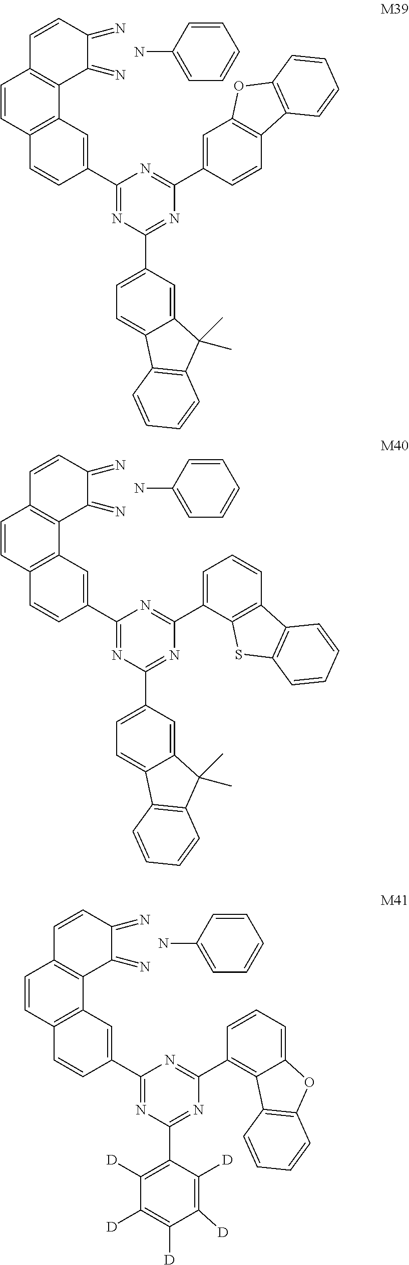 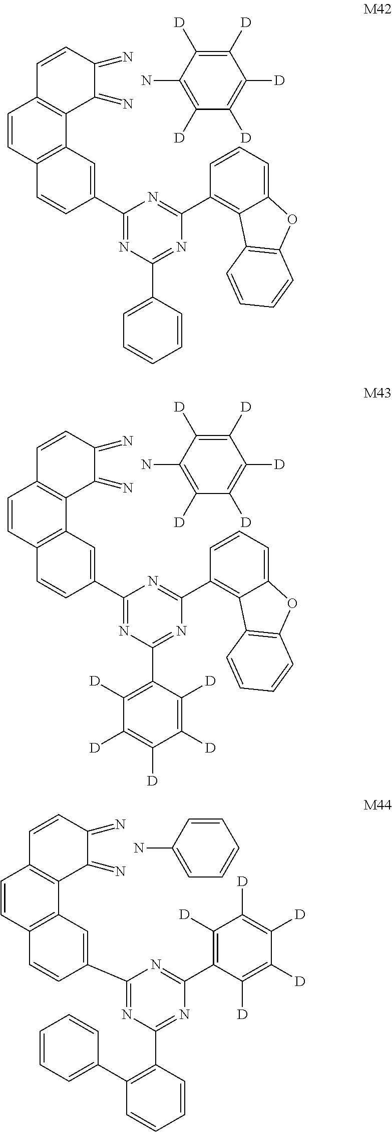 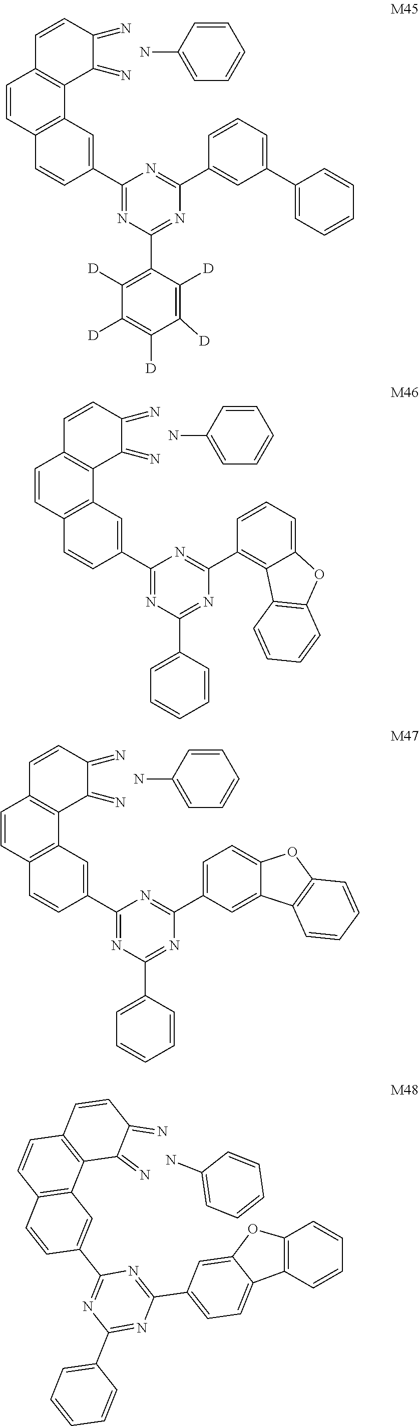 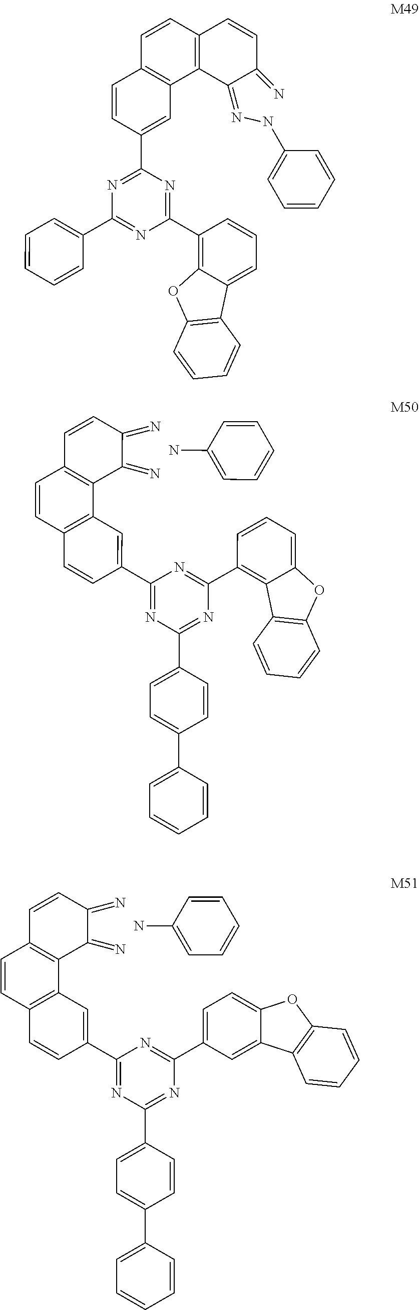 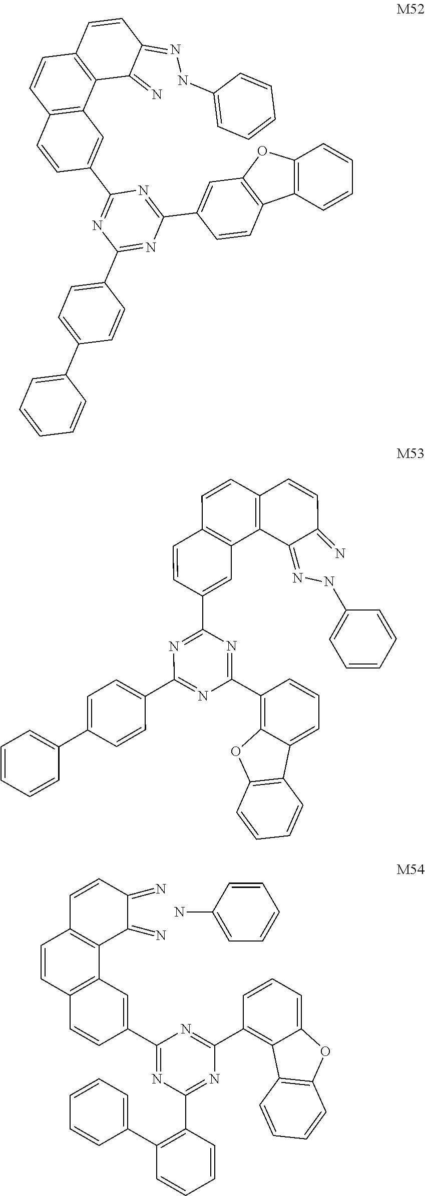 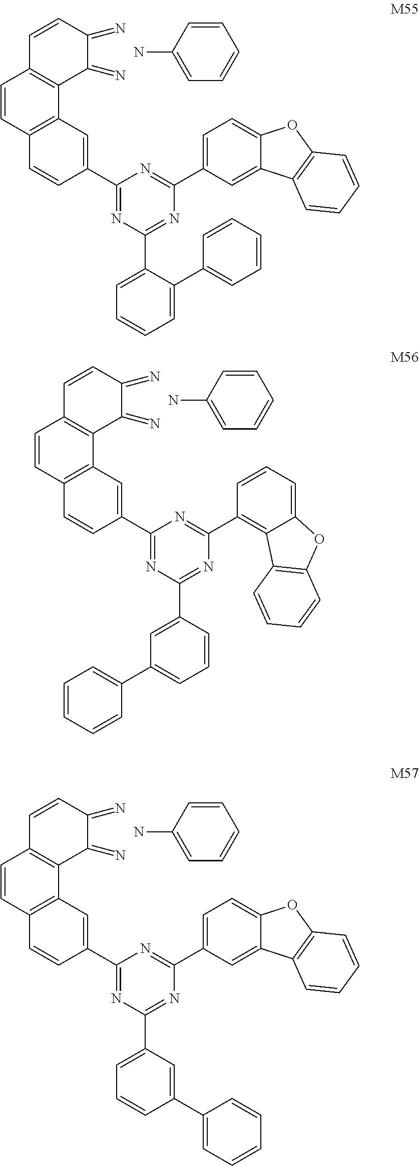 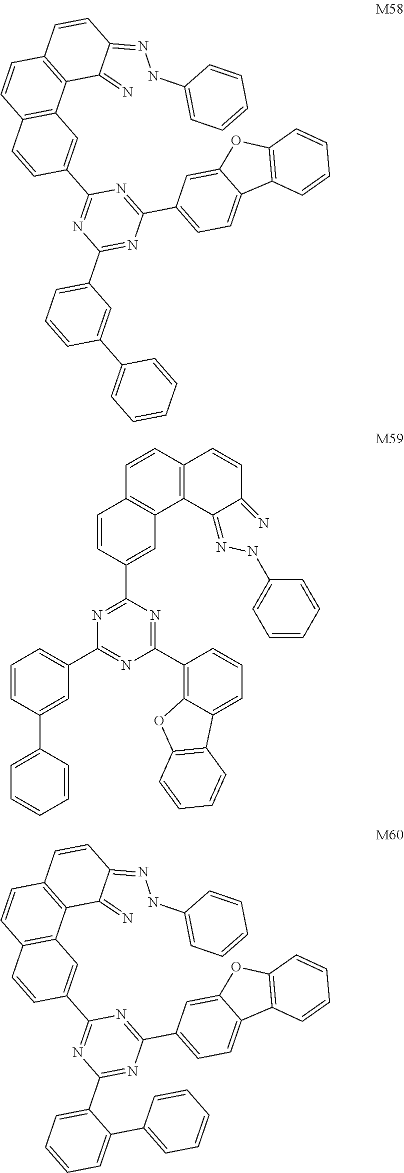 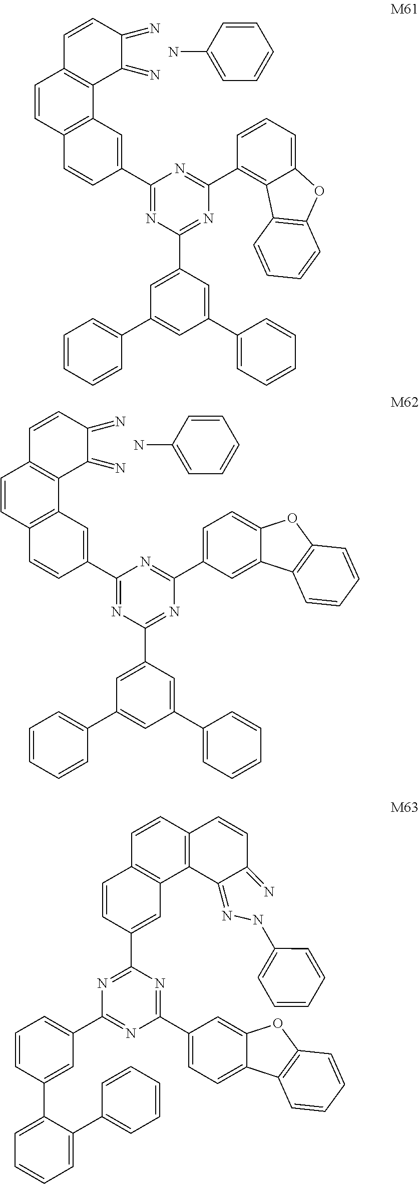 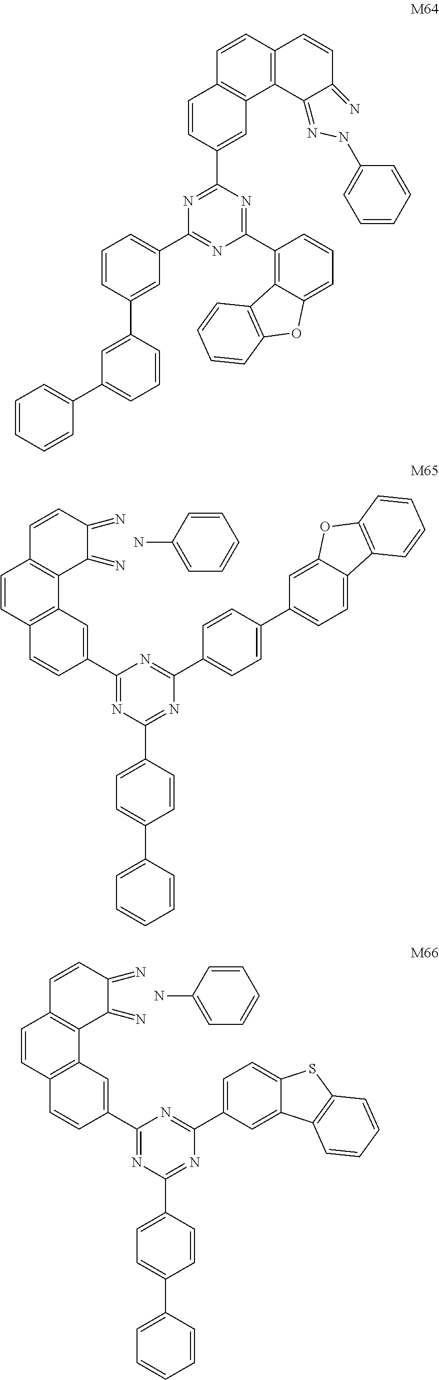 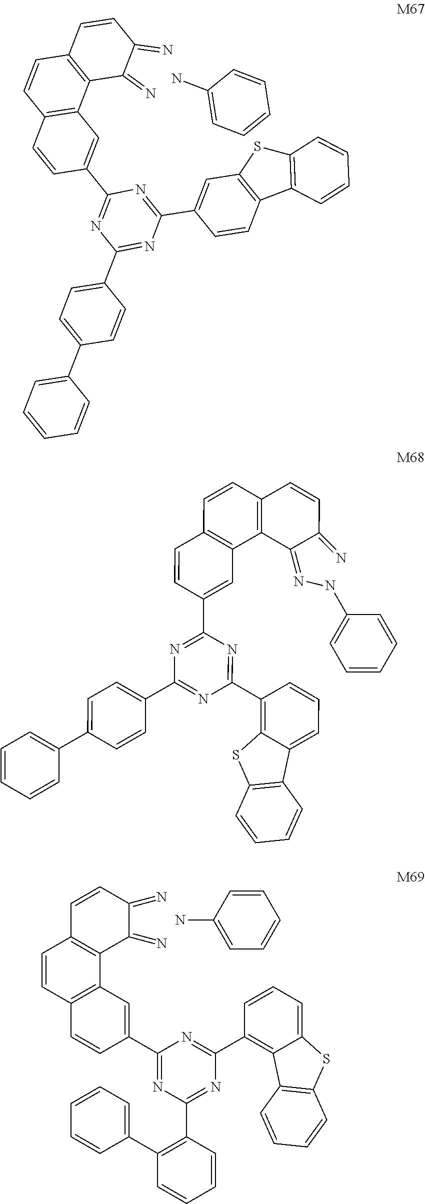 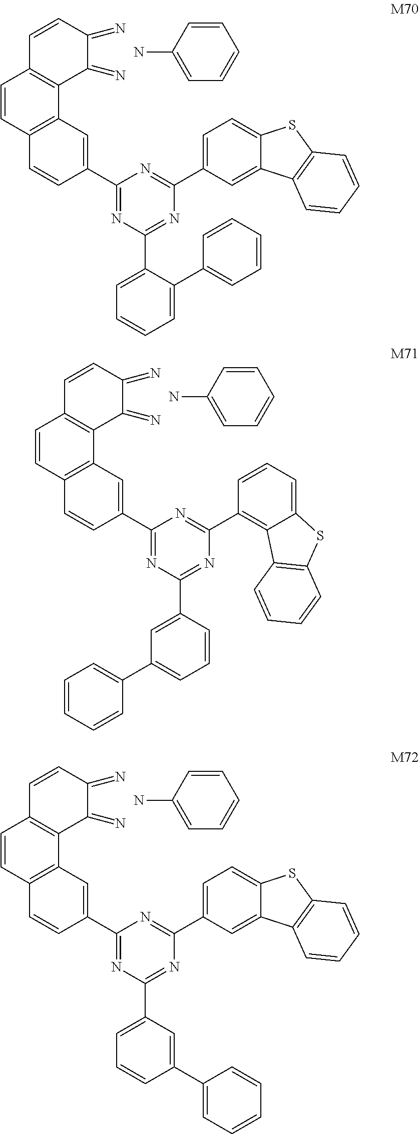 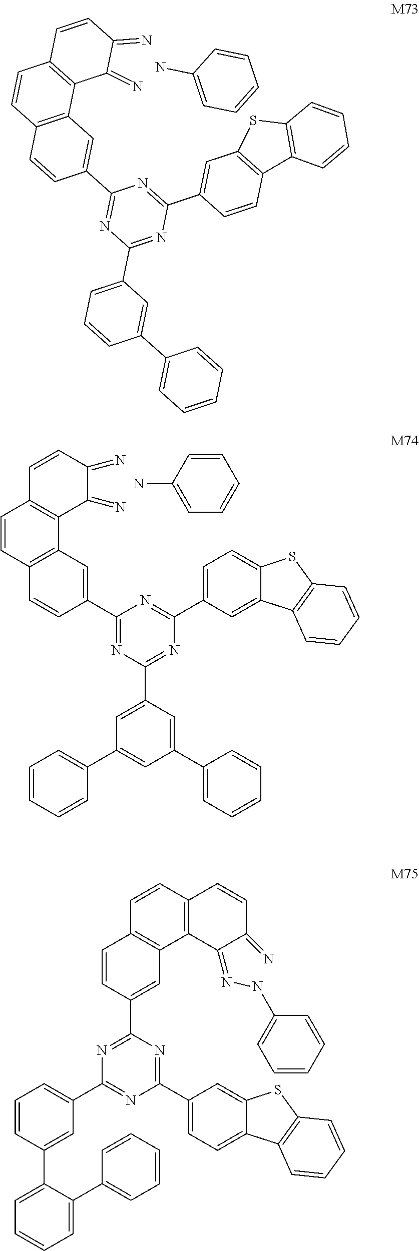 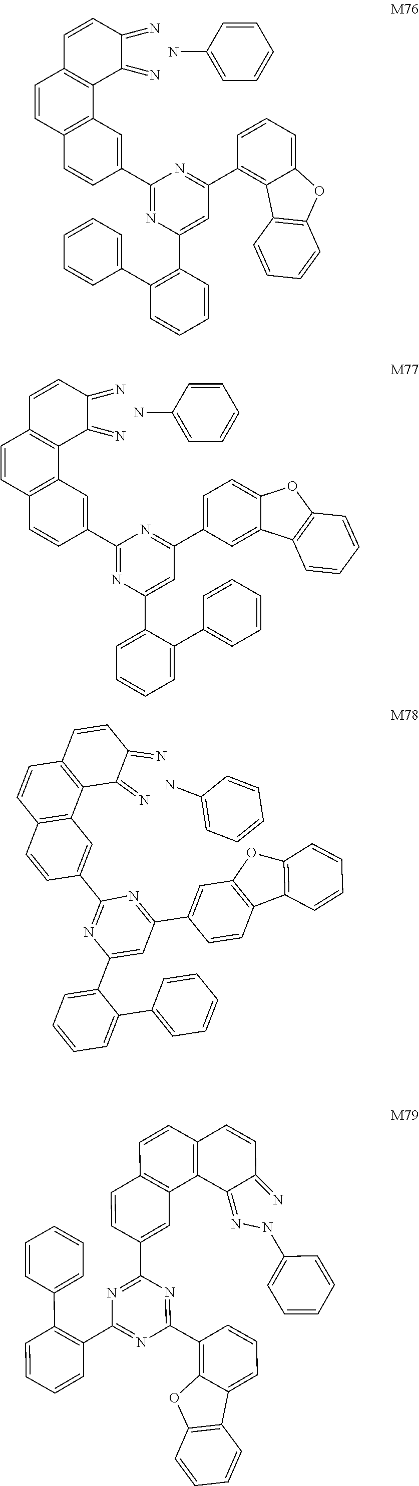 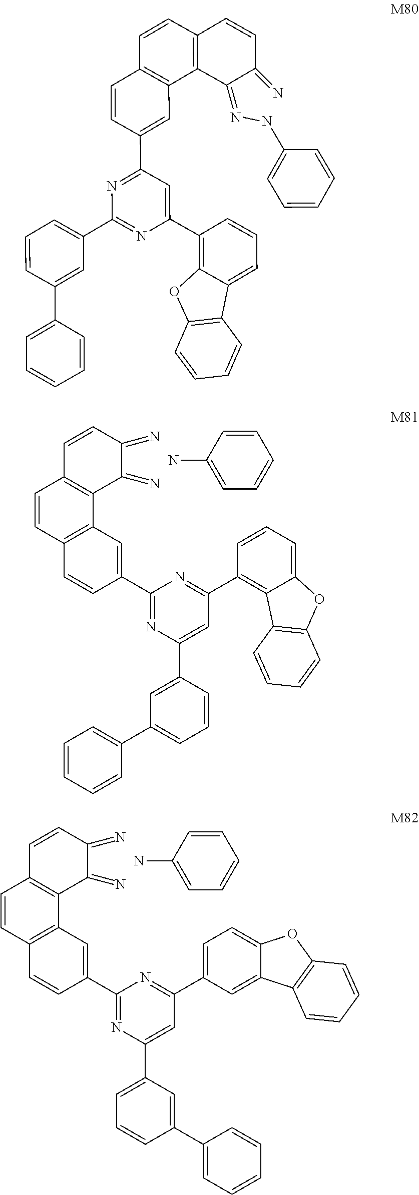 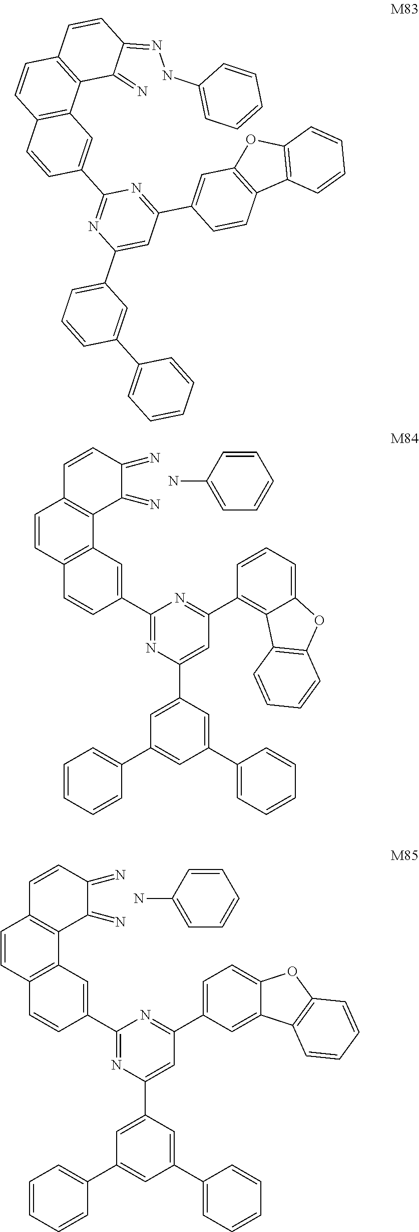 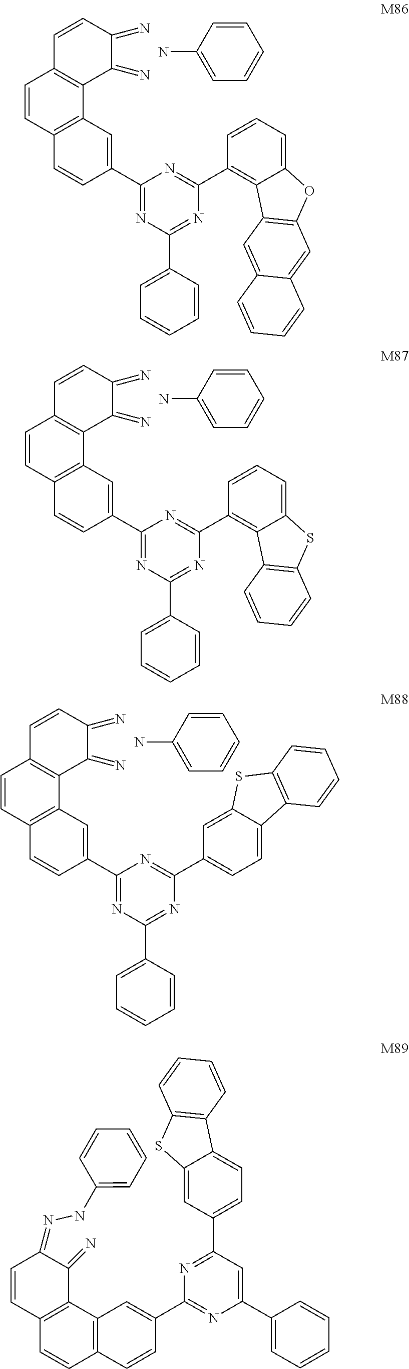 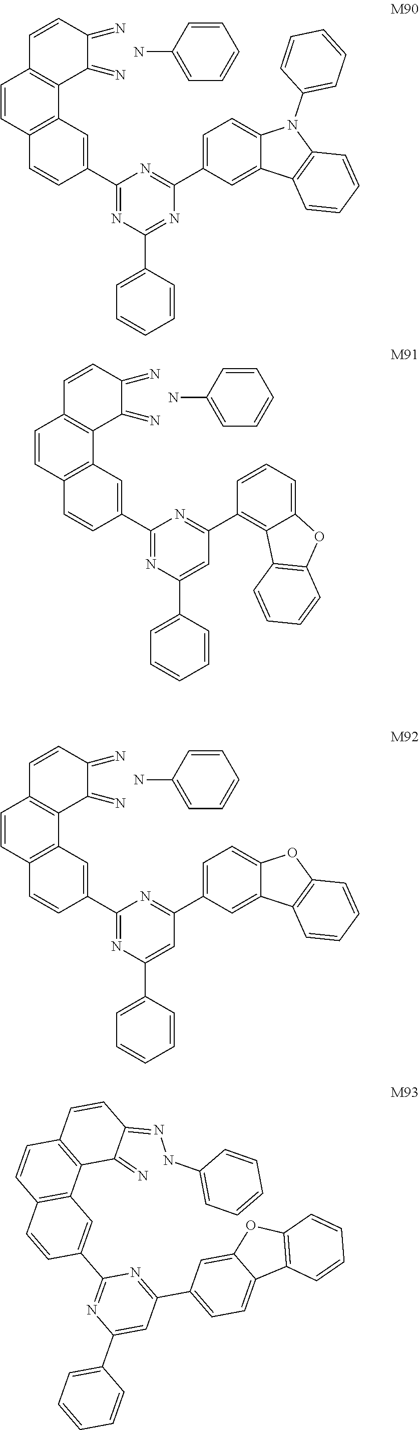 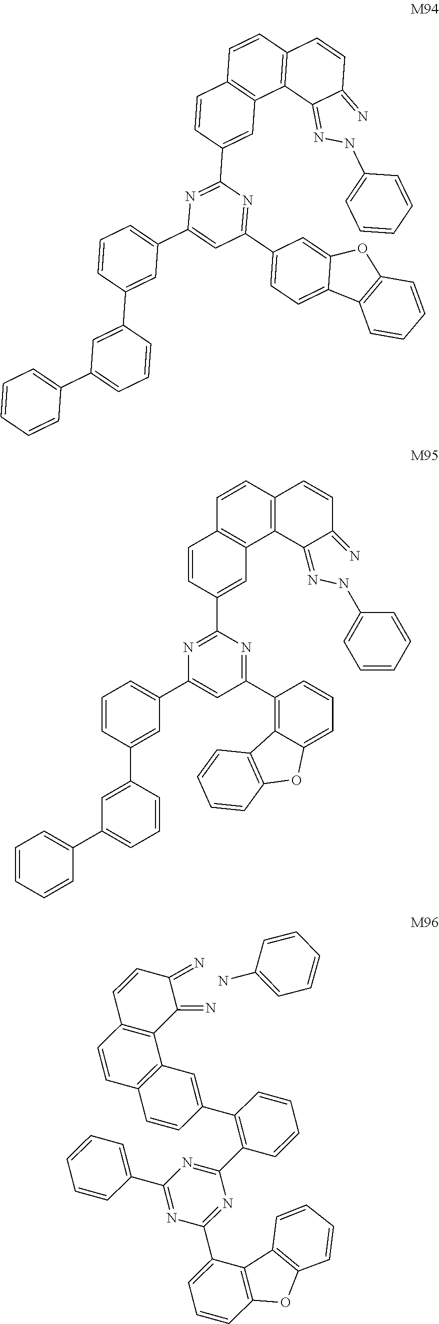 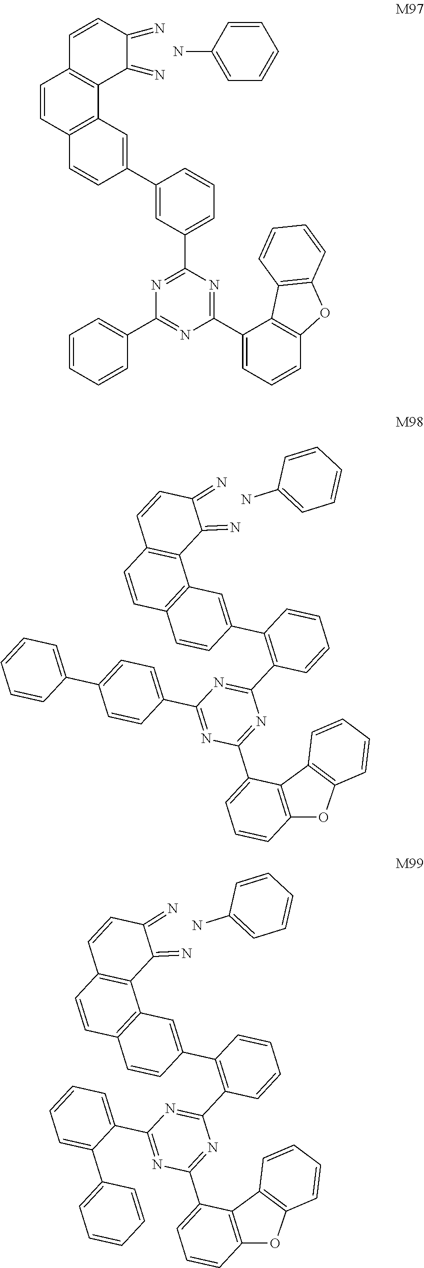 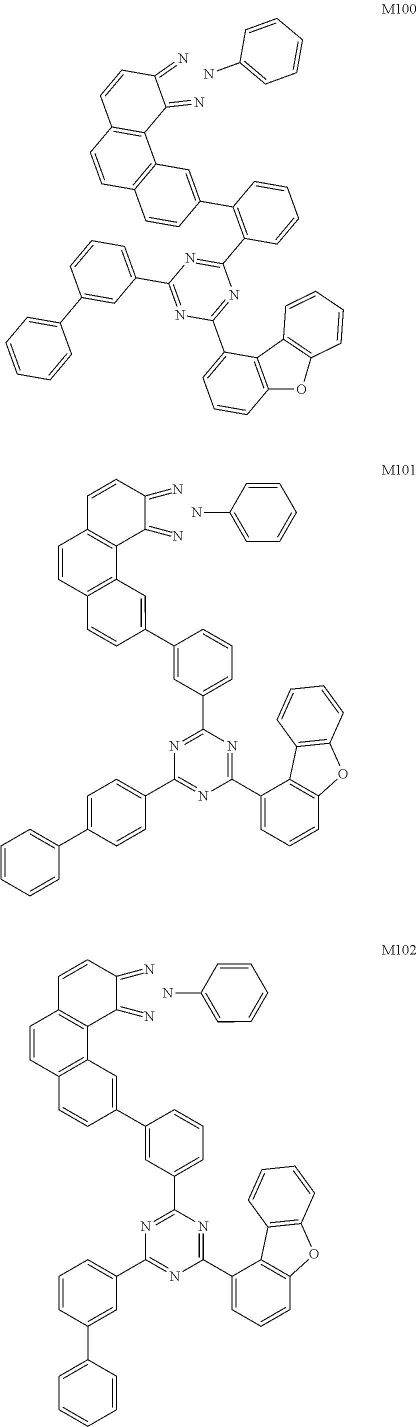 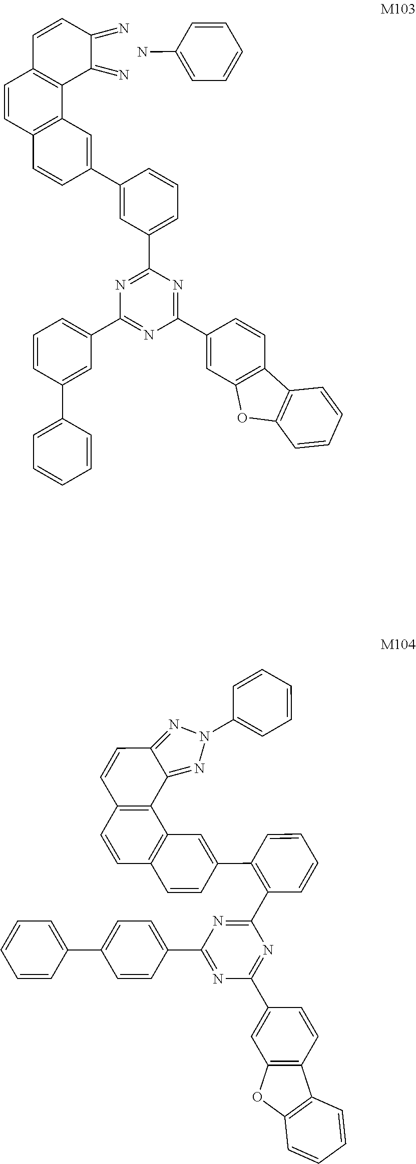 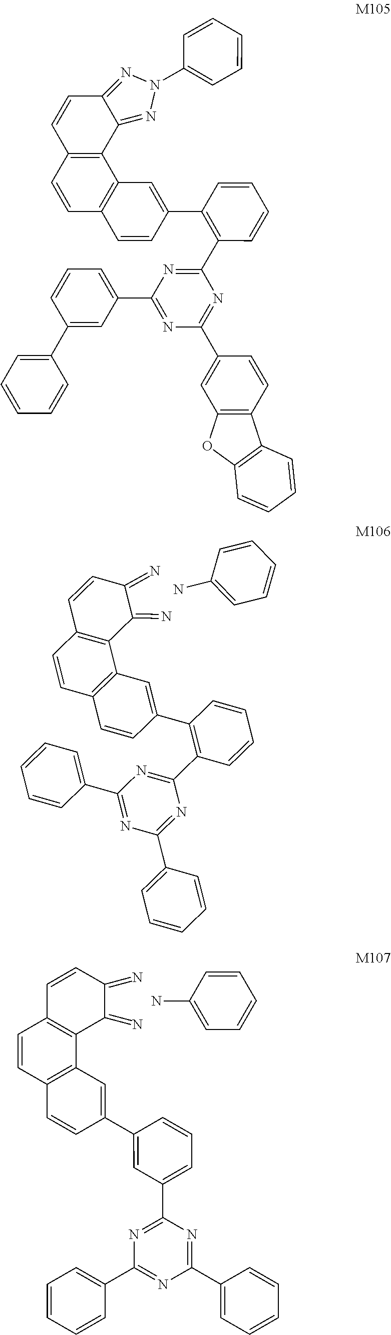 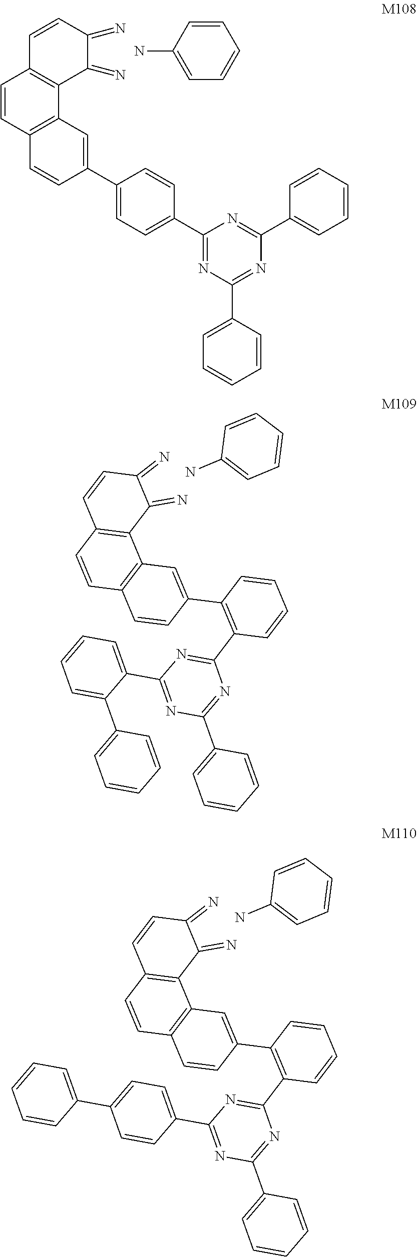 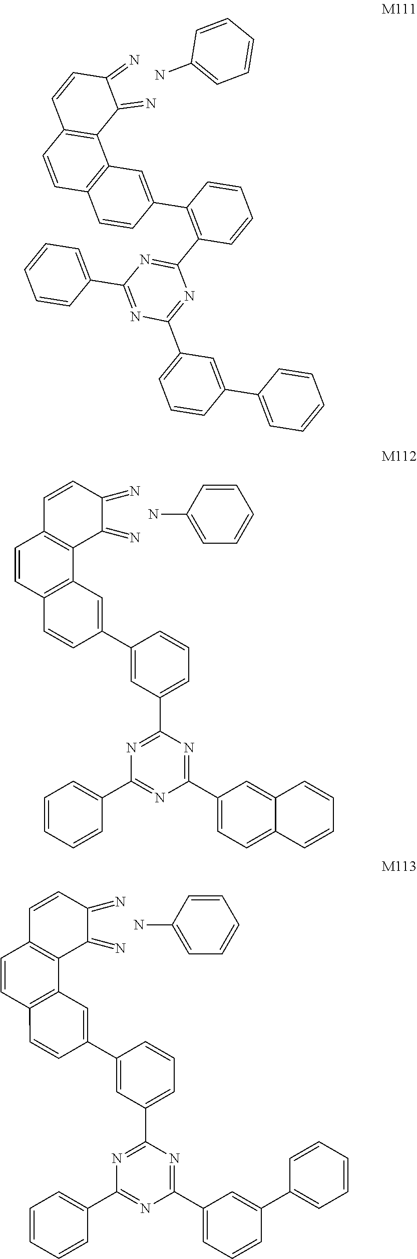 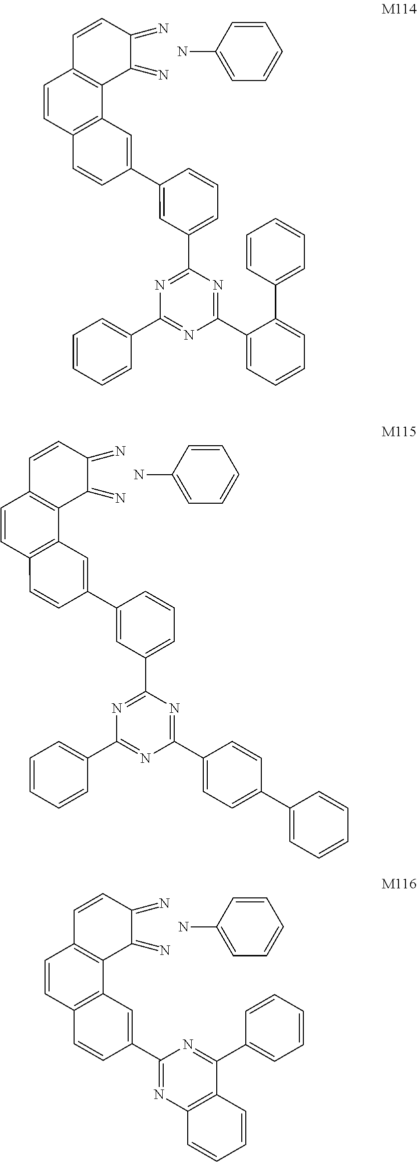 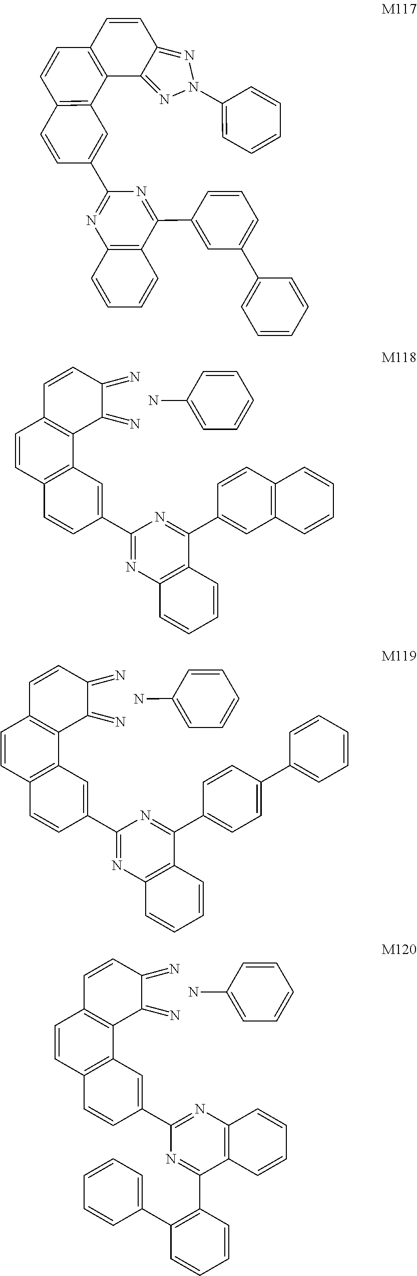 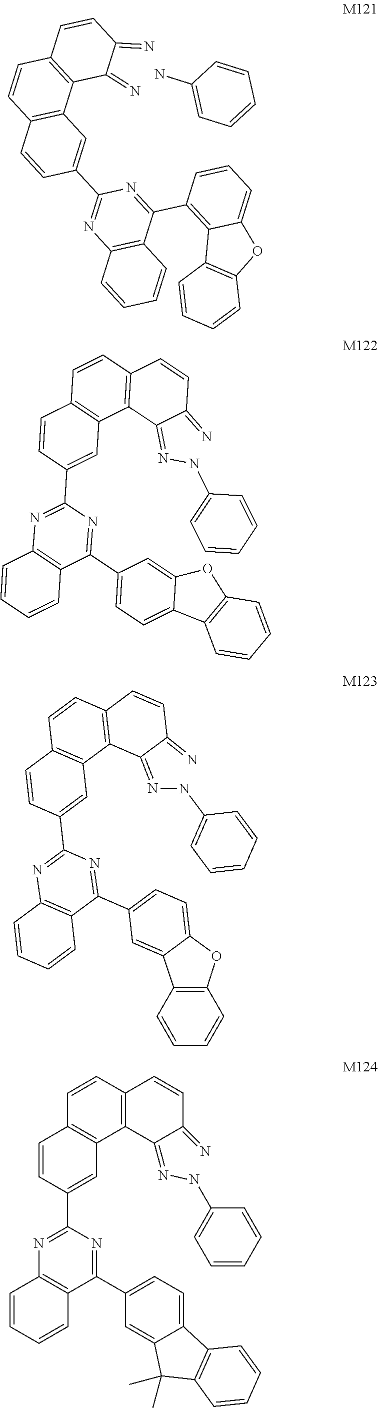 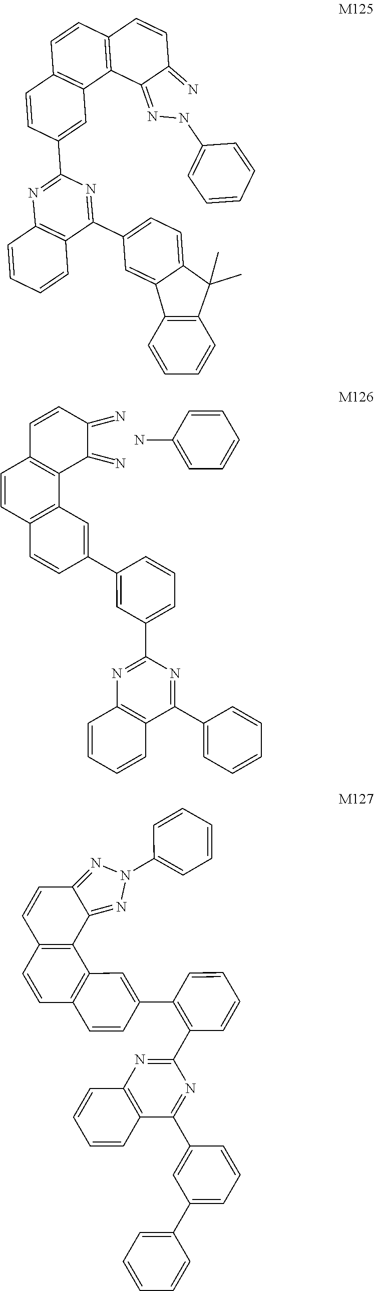 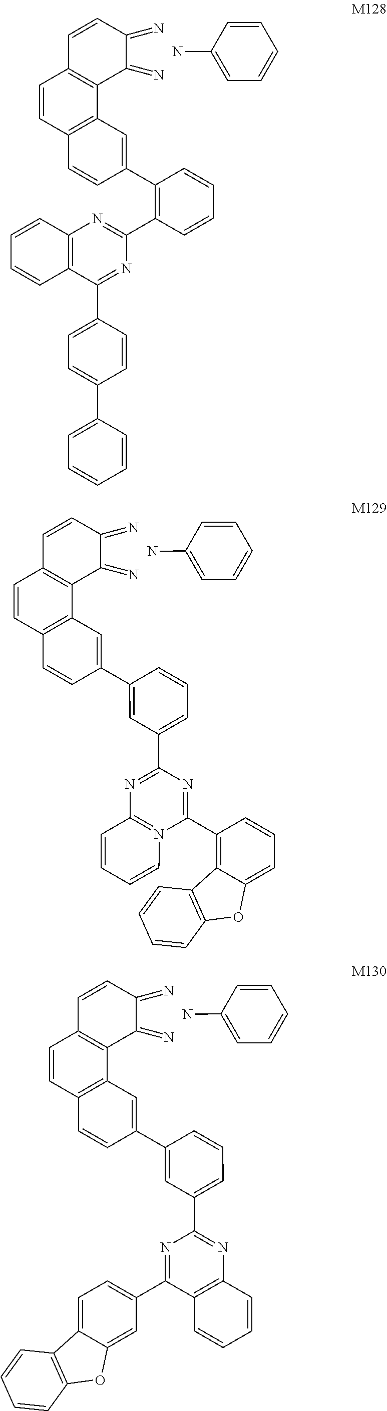 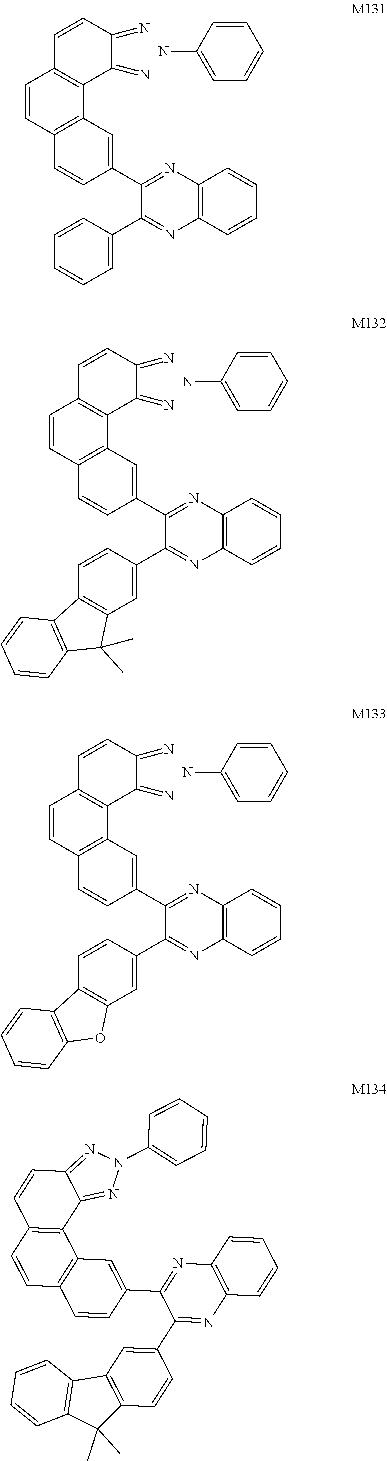 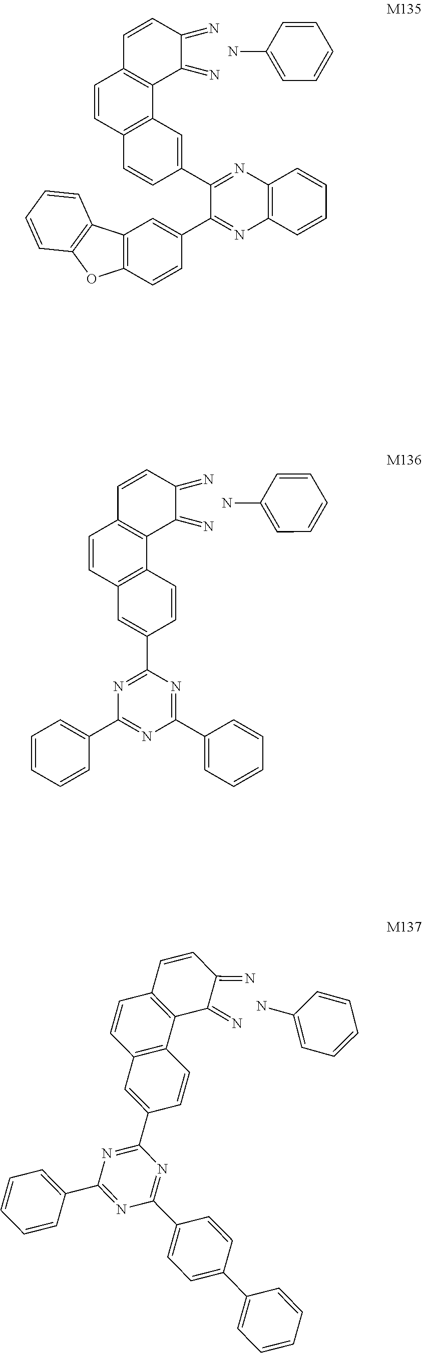 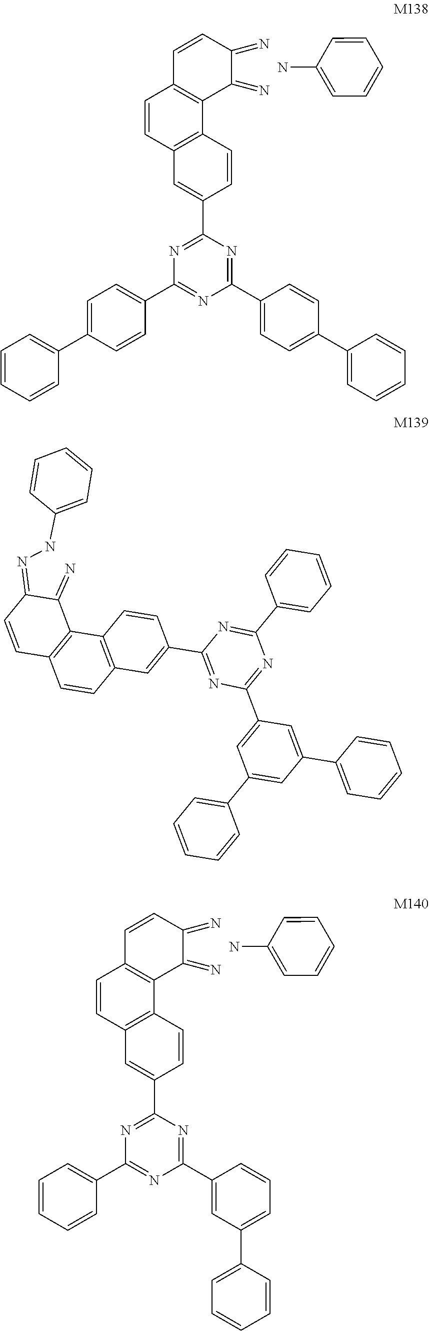 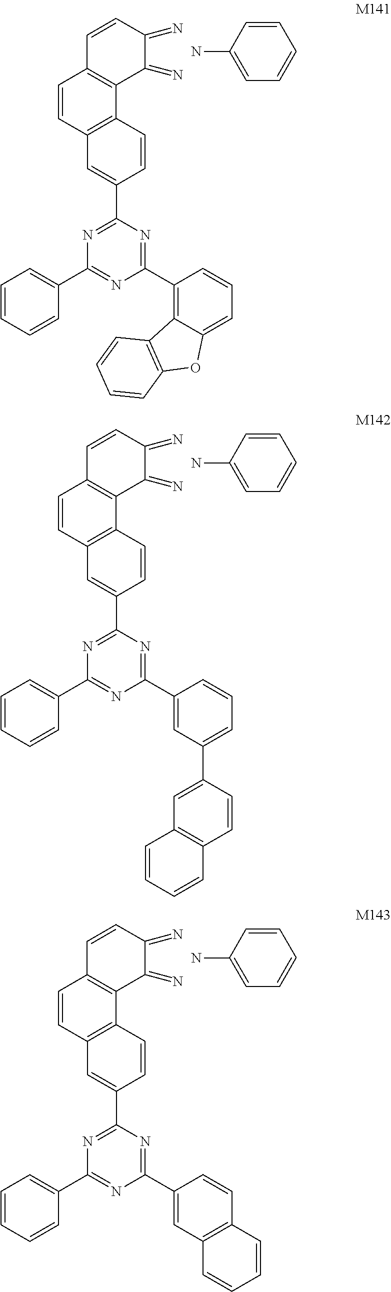 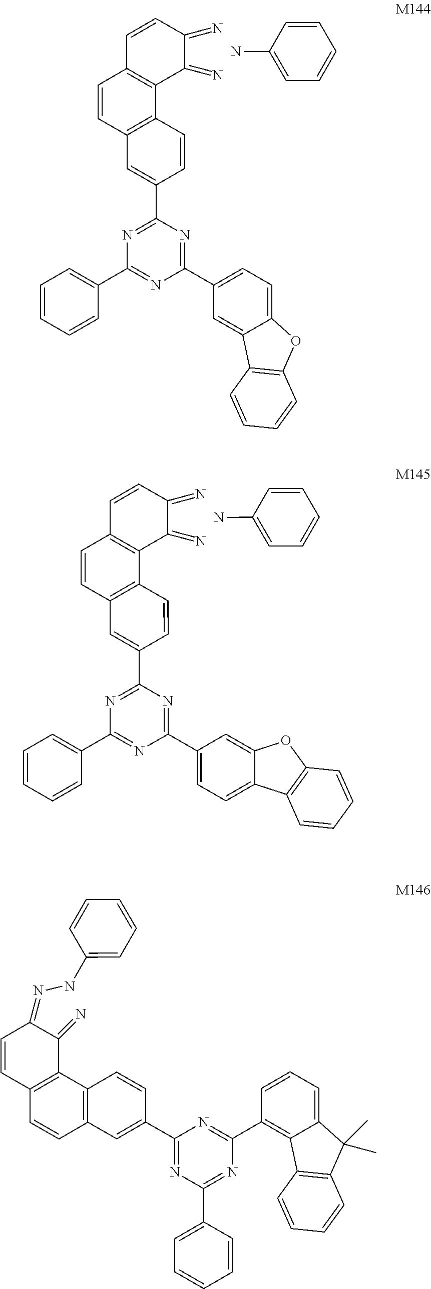 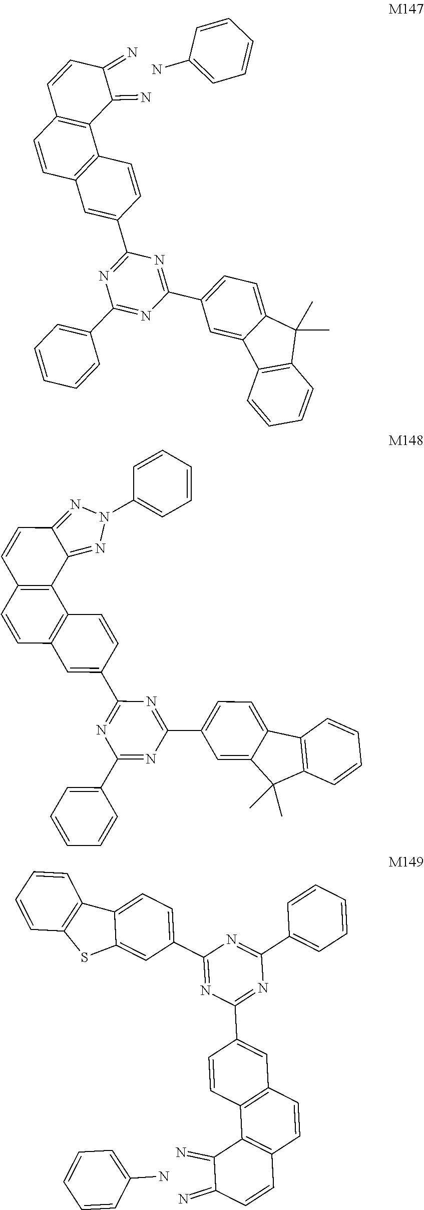 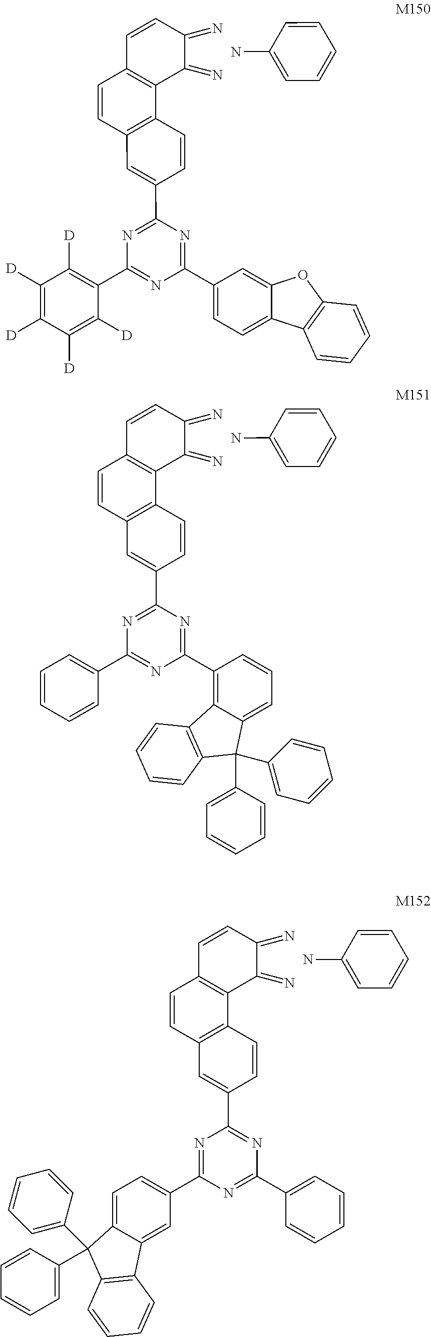 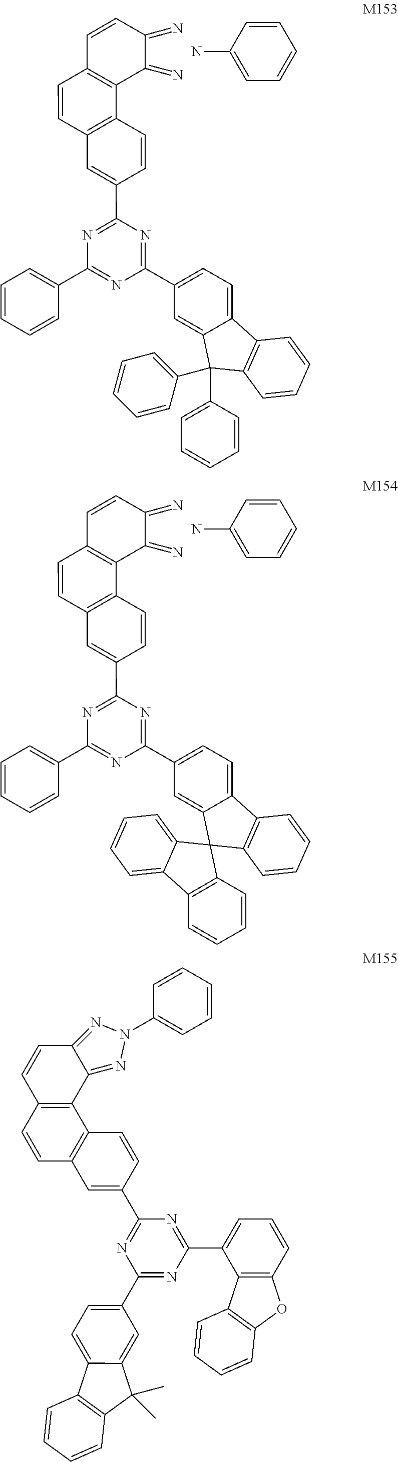 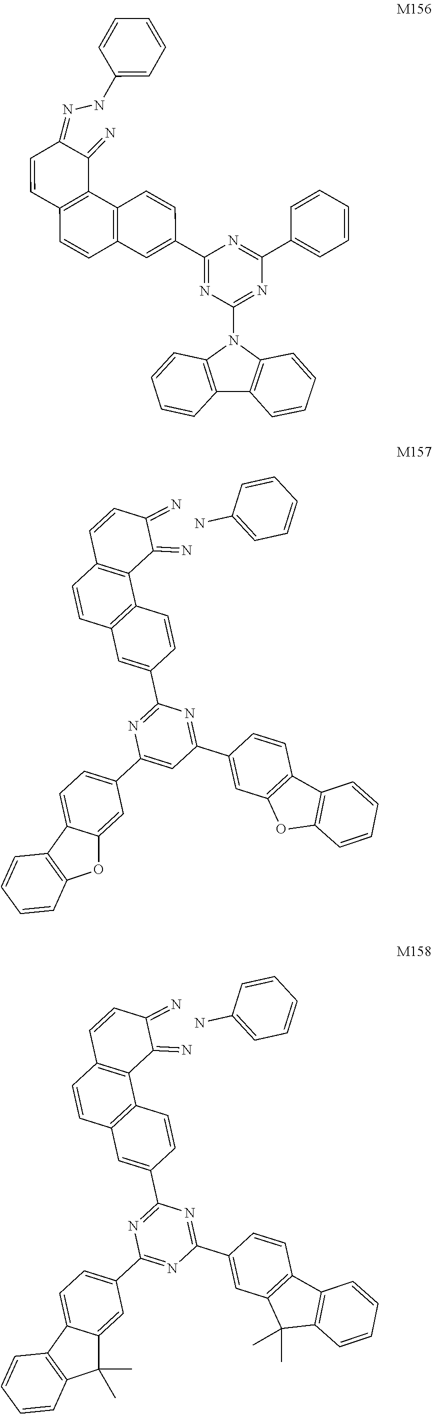 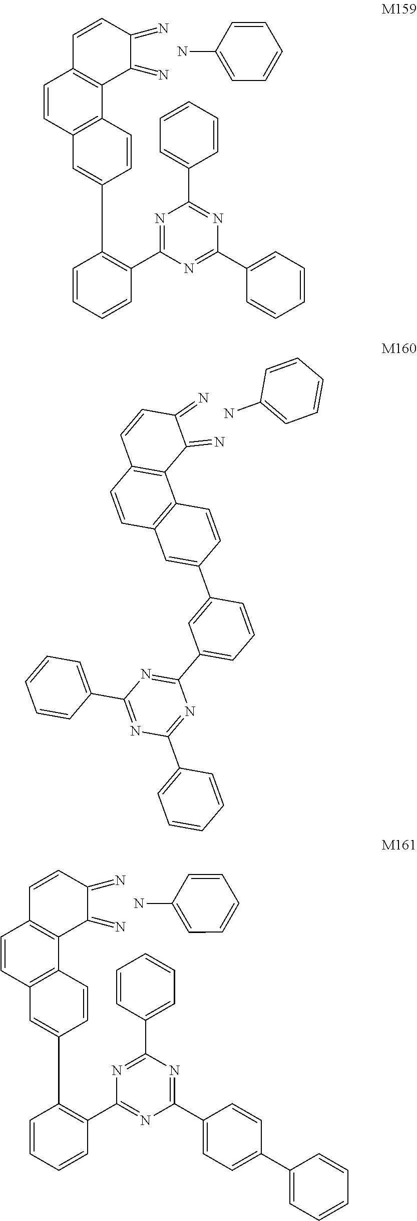 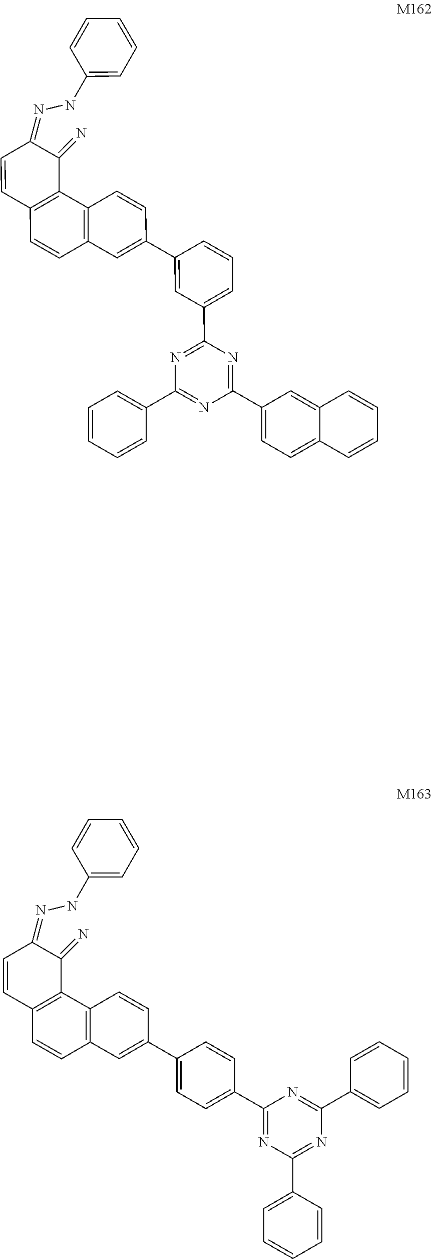 [SMILES: N=C1/C(=N\Nc2ccccc2)C=Cc2ccc3cc(-c4cccc(-c5nc(-c6ccccc6)nc(-c6ccc7ccccc7c6)n5)c4)ccc3c21 ; N=C1/C(=N\Nc2ccccc2)C=Cc2ccc3cc(-c4ccc(-c5nc(-c6ccccc6)nc(-c6ccccc6)n5)cc4)ccc3c21] 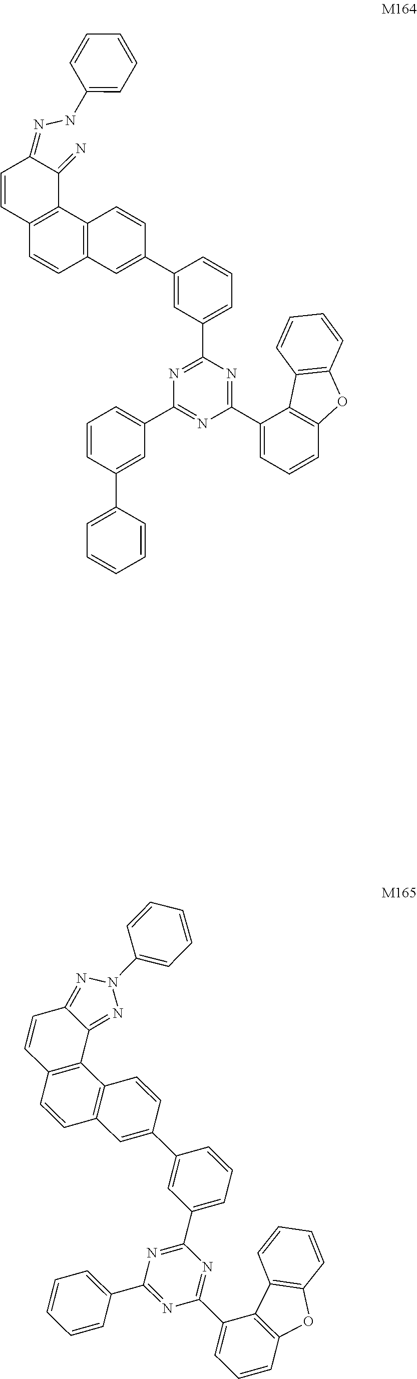 [SMILES: N=C1/C(=N\Nc2ccccc2)C=Cc2ccc3cc(-c4cccc(-c5nc(-c6cccc(-c7ccccc7)c6)nc(-c6cccc7oc8ccccc8c67)n5)c4)ccc3c21 ; c1ccc(-c2nc(-c3cccc(-c4ccc5c(ccc6ccc7nn(-c8ccccc8)nc7c65)c4)c3)nc(-c3cccc4oc5ccccc5c34)n2)cc1] 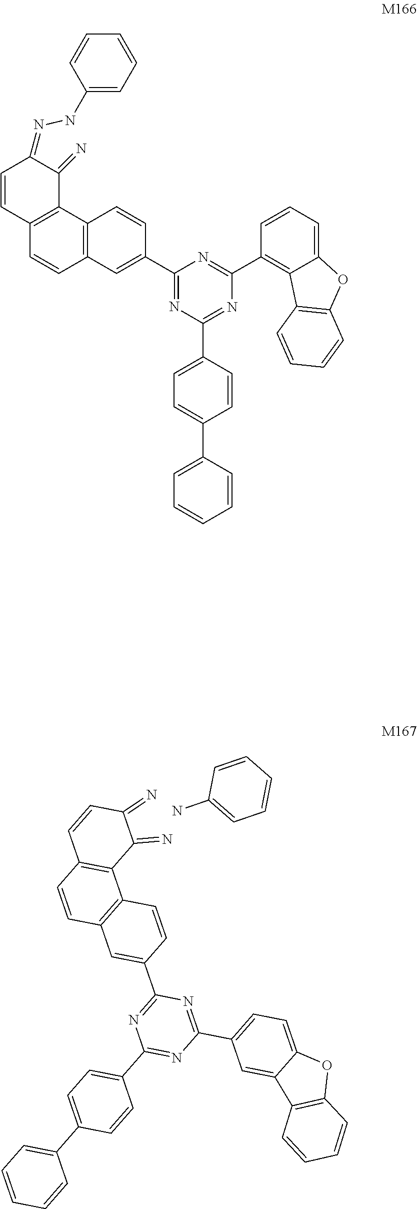 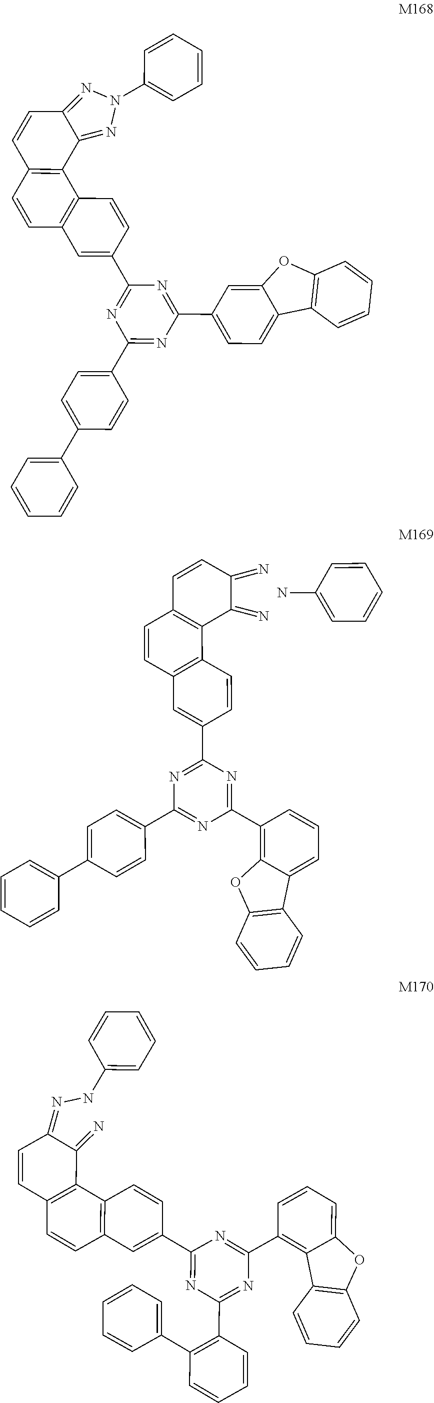 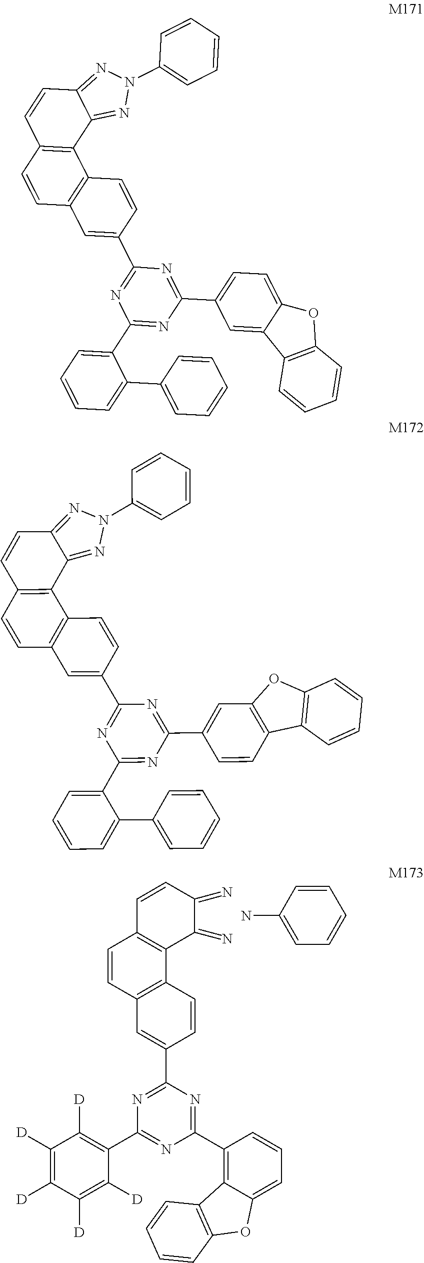 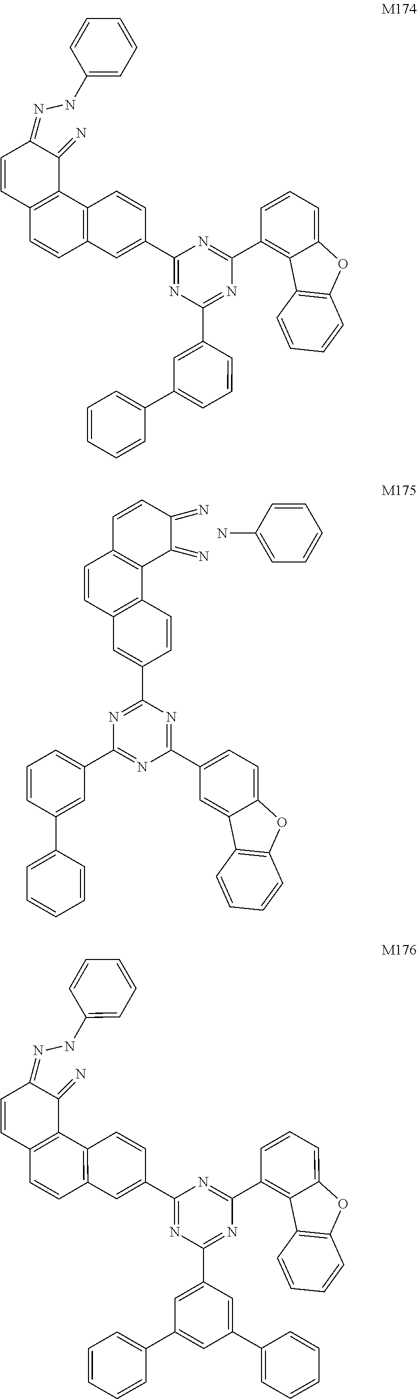 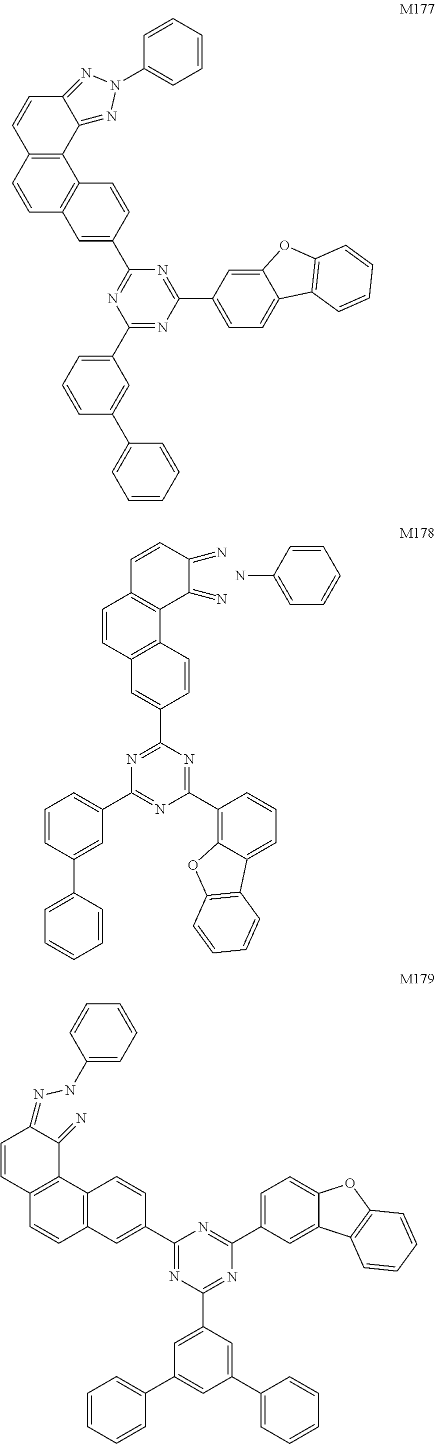 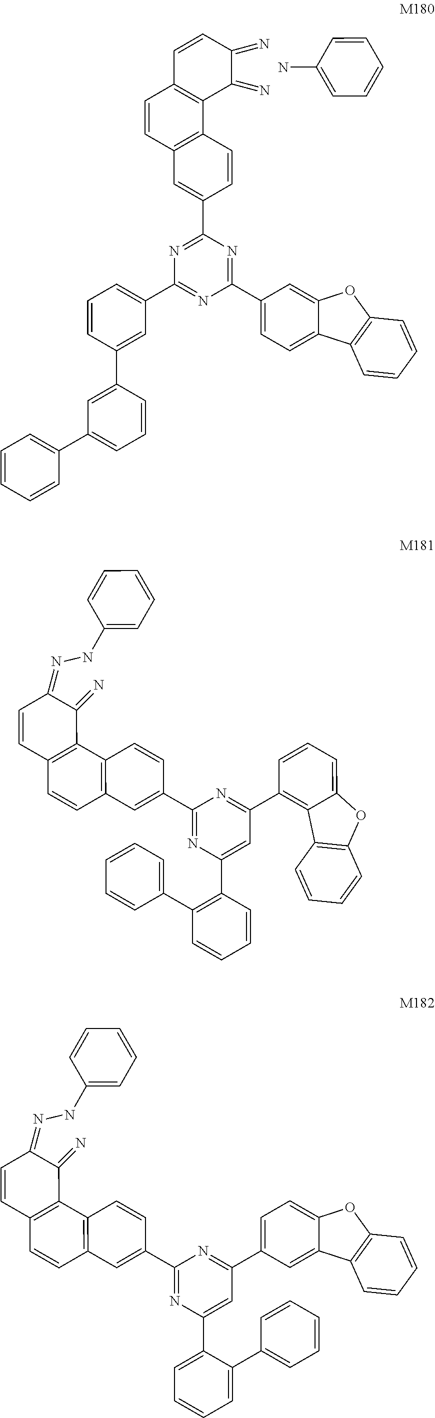 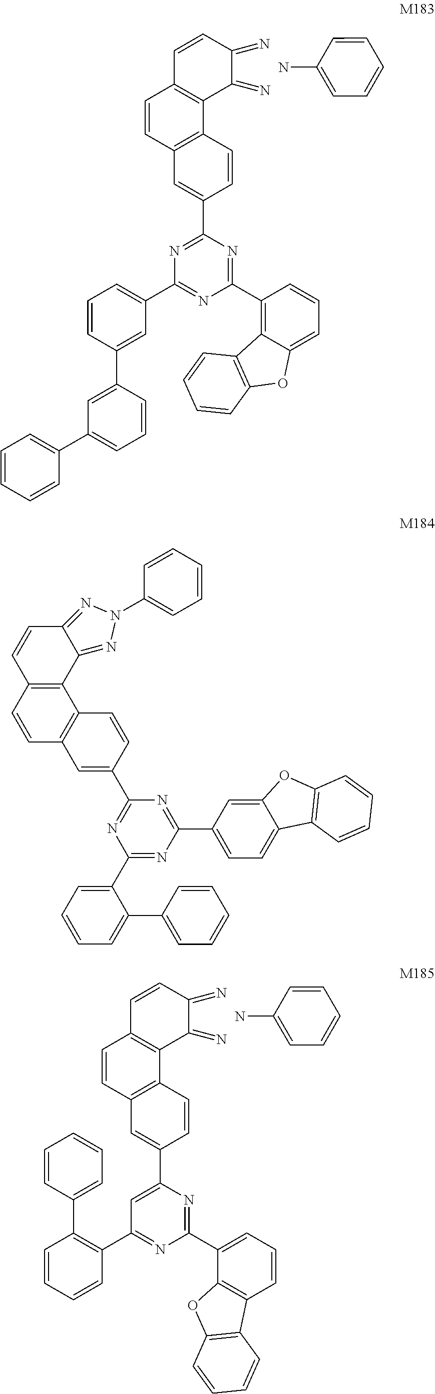 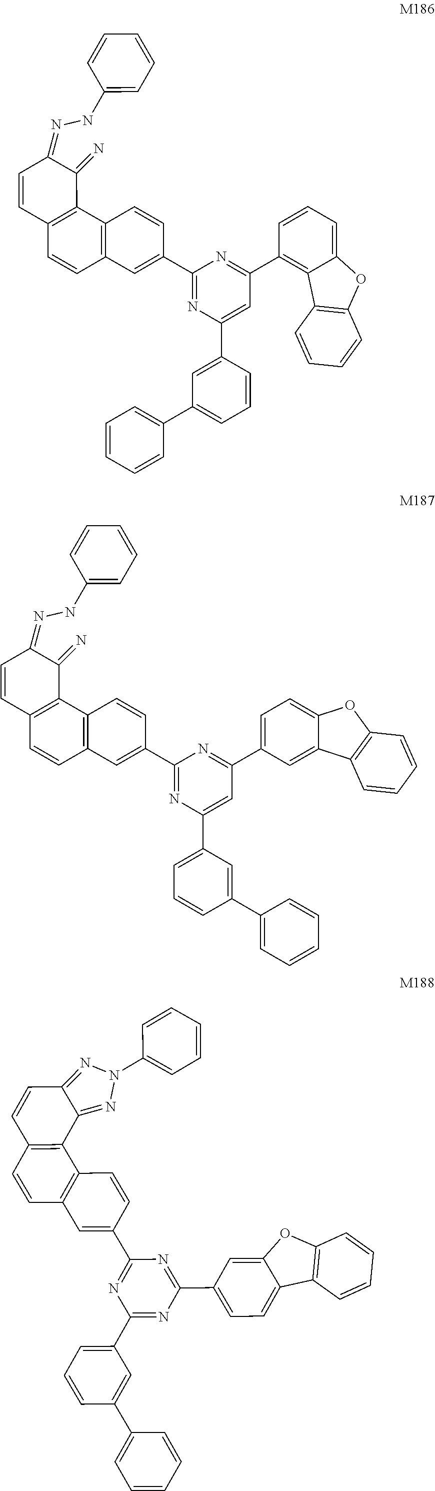 [SMILES: N=C1/C(=N\Nc2ccccc2)C=Cc2ccc3cc(-c4nc(-c5cccc(-c6ccccc6)c5)cc(-c5cccc6oc7ccccc7c56)n4)ccc3c21 ; N=C1/C(=N\Nc2ccccc2)C=Cc2ccc3cc(-c4nc(-c5cccc(-c6ccccc6)c5)cc(-c5ccc6oc7ccccc7c6c5)n4)ccc3c21 ; c1ccc(-c2cccc(-c3nc(-c4ccc5c(ccc6ccc7nn(-c8ccccc8)nc7c65)c4)nc(-c4ccc5c(c4)oc4ccccc45)n3)c2)cc1] 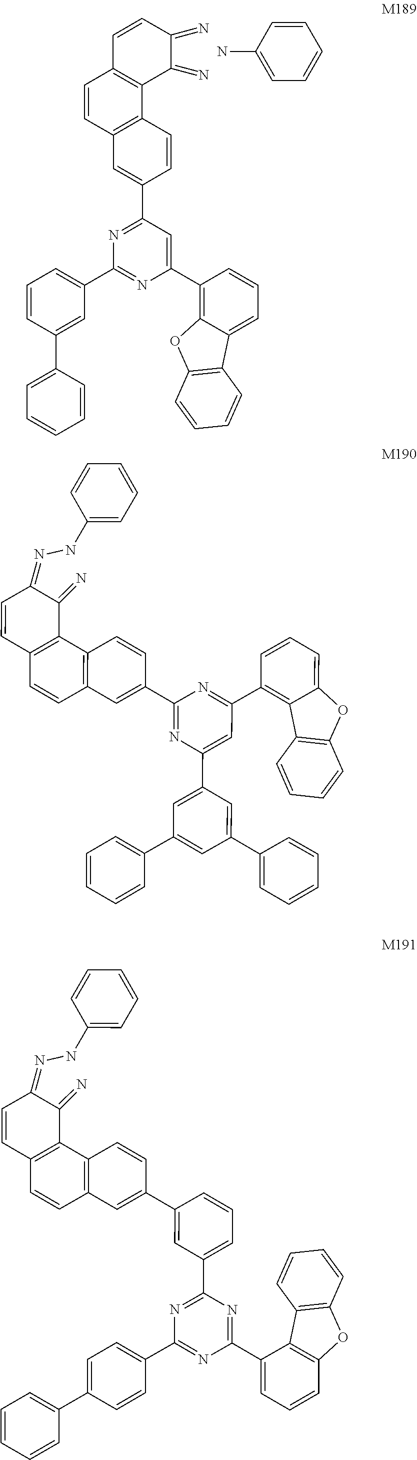 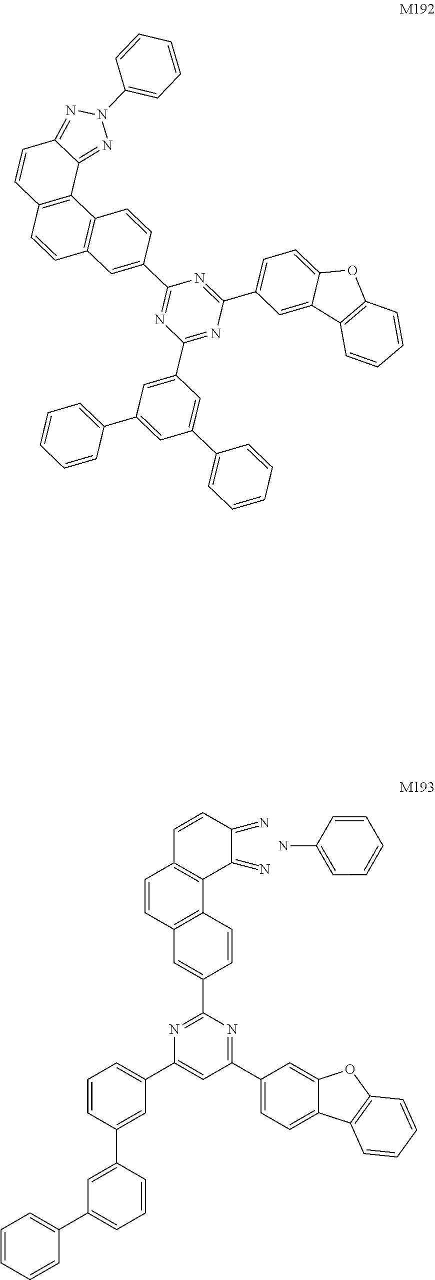 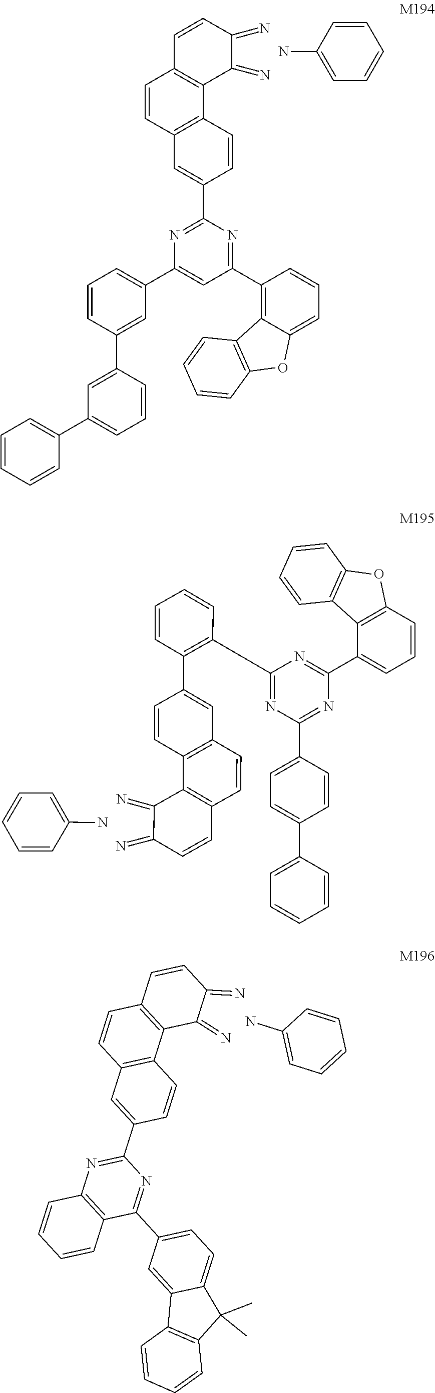 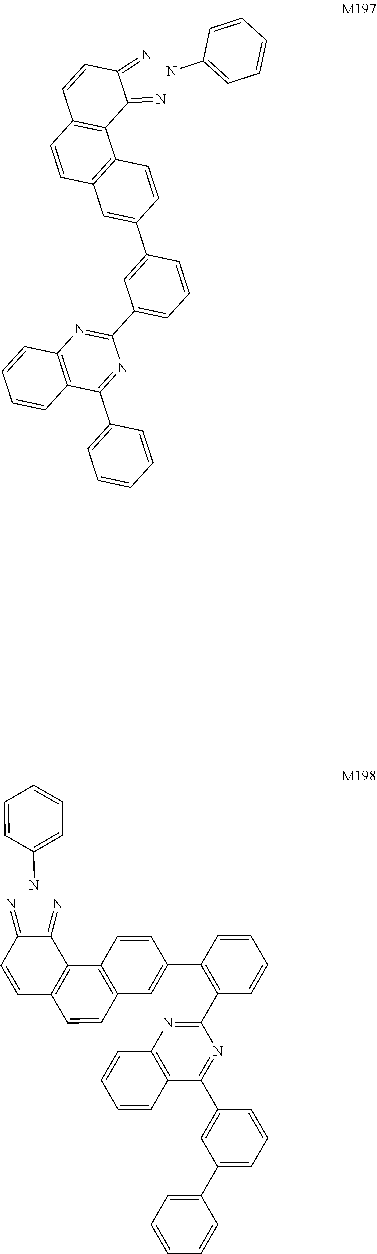 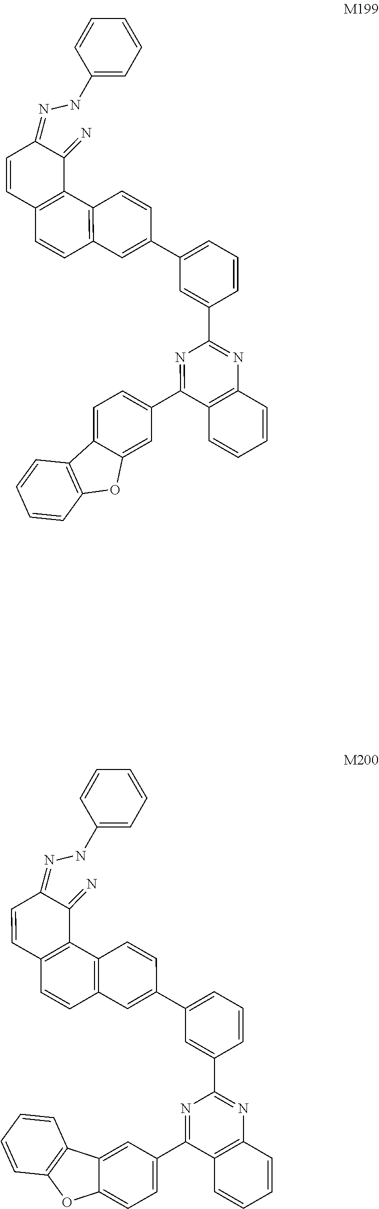 [SMILES: N=C1/C(=N\Nc2ccccc2)C=Cc2ccc3cc(-c4cccc(-c5nc(-c6ccc7c(c6)oc6ccccc67)c6ccccc6n5)c4)ccc3c21 ; N=C1/C(=N\Nc2ccccc2)C=Cc2ccc3cc(-c4cccc(-c5nc(-c6ccc7oc8ccccc8c7c6)c6ccccc6n5)c4)ccc3c21] 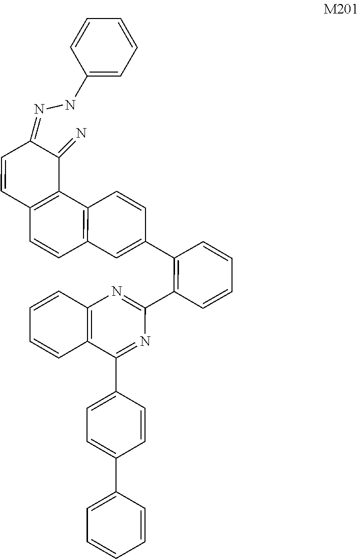 [SMILES: N=C1/C(=N\Nc2ccccc2)C=Cc2ccc3cc(-c4ccccc4-c4nc(-c5ccc(-c6ccccc6)cc5)c5ccccc5n4)ccc3c21] 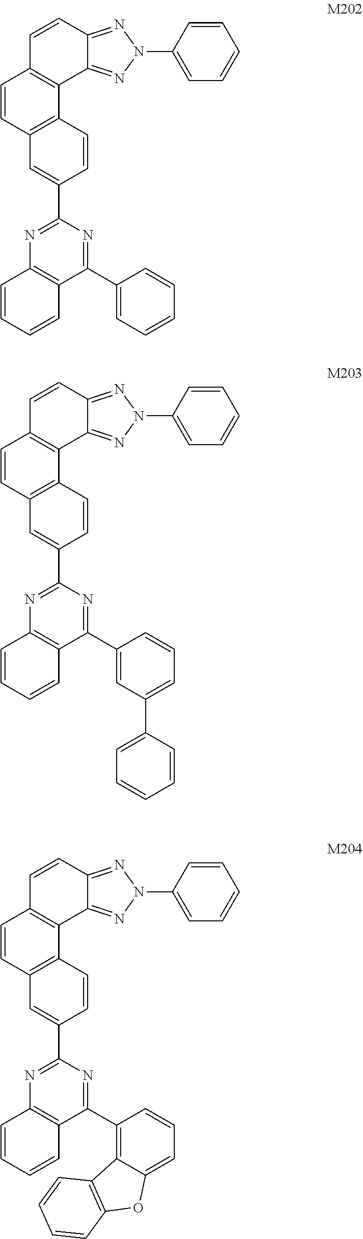 [SMILES: c1ccc(-c2nc(-c3ccc4c(ccc5ccc6nn(-c7ccccc7)nc6c54)c3)nc3ccccc23)cc1 ; c1ccc(-c2cccc(-c3nc(-c4ccc5c(ccc6ccc7nn(-c8ccccc8)nc7c65)c4)nc4ccccc34)c2)cc1 ; c1ccc(-n2nc3ccc4ccc5cc(-c6nc(-c7cccc8oc9ccccc9c78)c7ccccc7n6)ccc5c4c3n2)cc1] 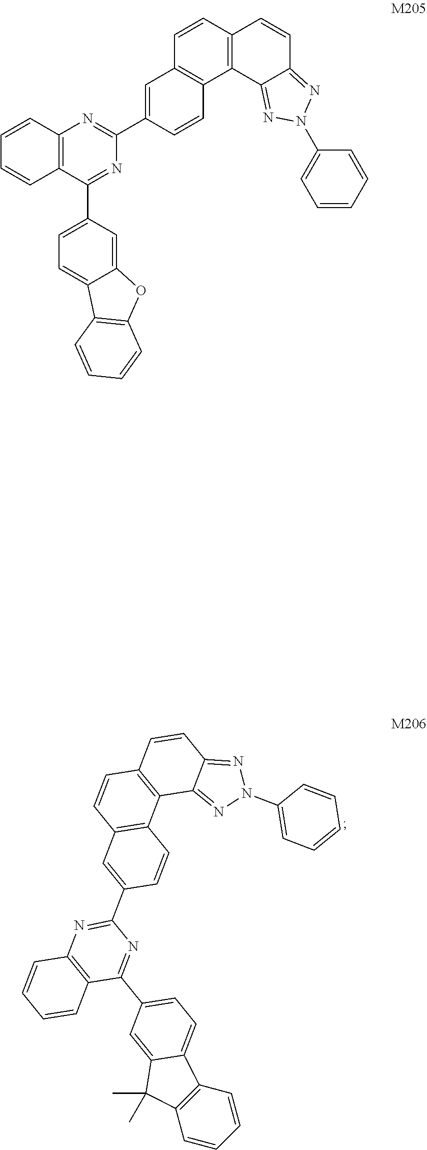 [SMILES: c1ccc(-n2nc3ccc4ccc5cc(-c6nc(-c7ccc8c(c7)oc7ccccc78)c7ccccc7n6)ccc5c4c3n2)cc1 ; CC1(C)c2ccccc2-c2ccc(-c3nc(-c4ccc5c(ccc6ccc7nn(-c8ccccc8)nc7c65)c4)nc4ccccc34)cc21]
and the compound N is an one of compounds N1 to N115: 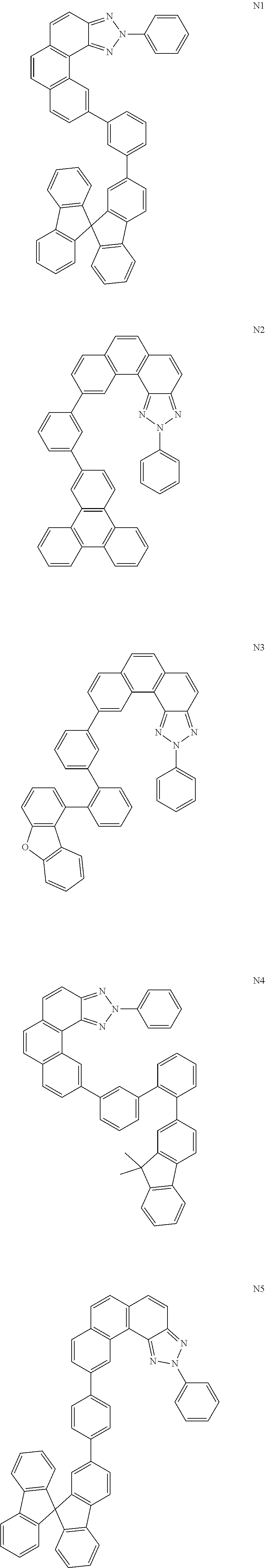 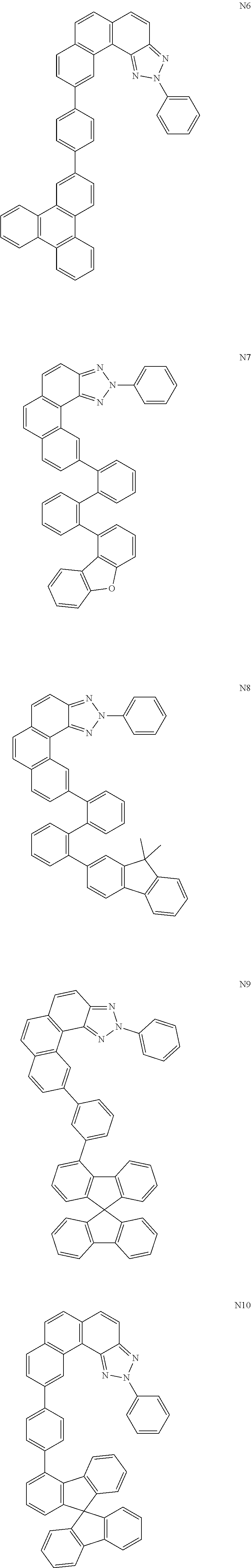 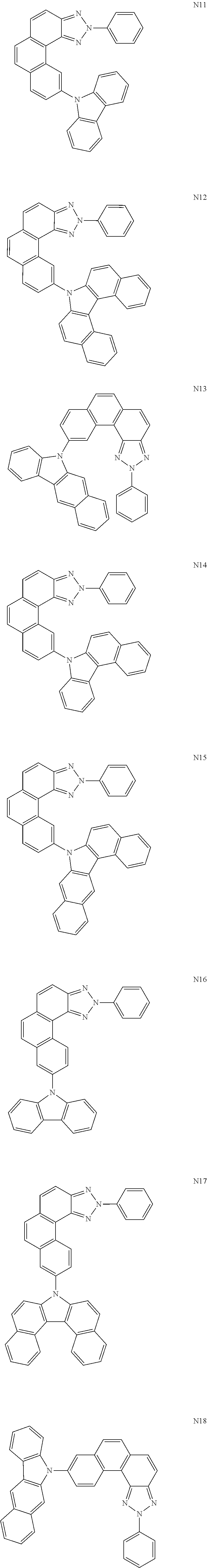 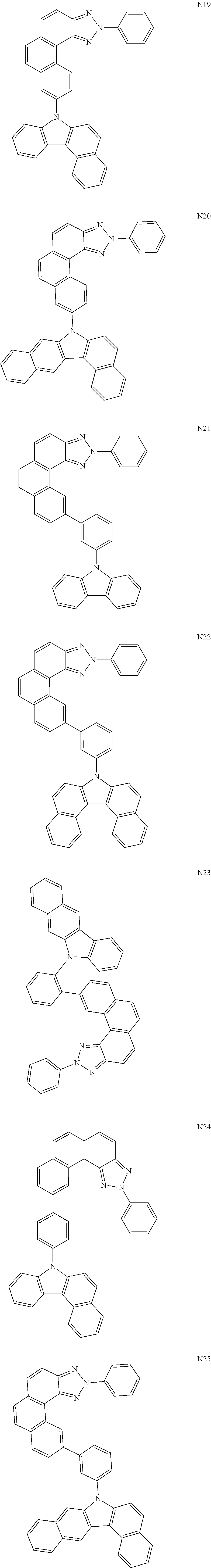 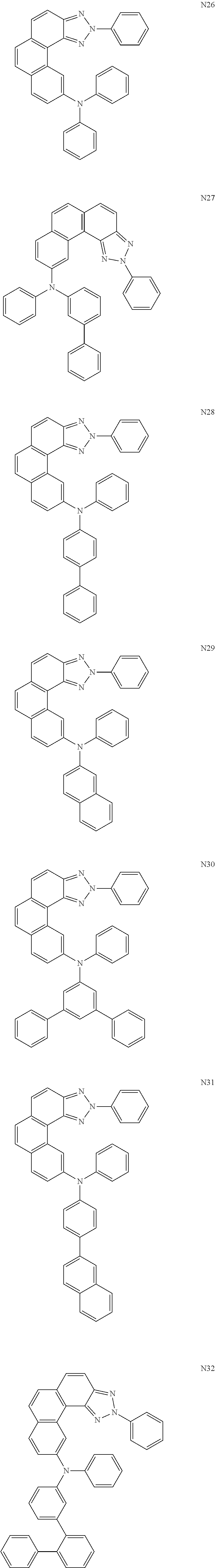 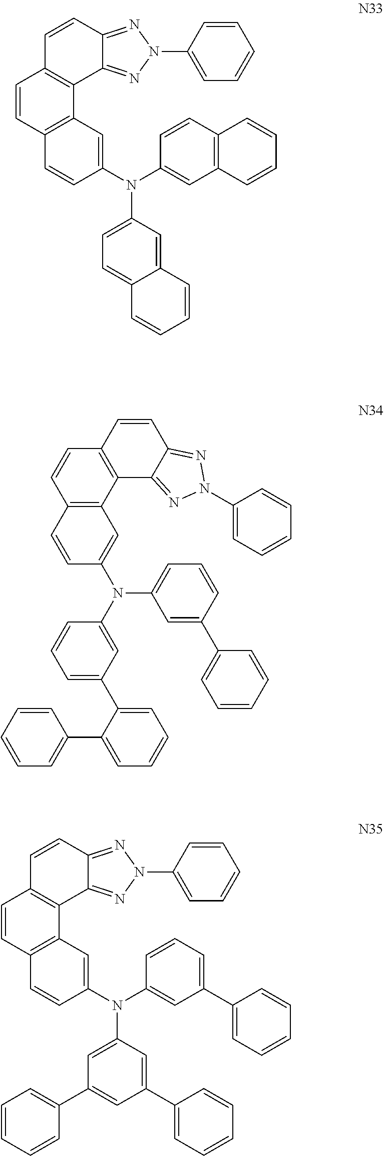 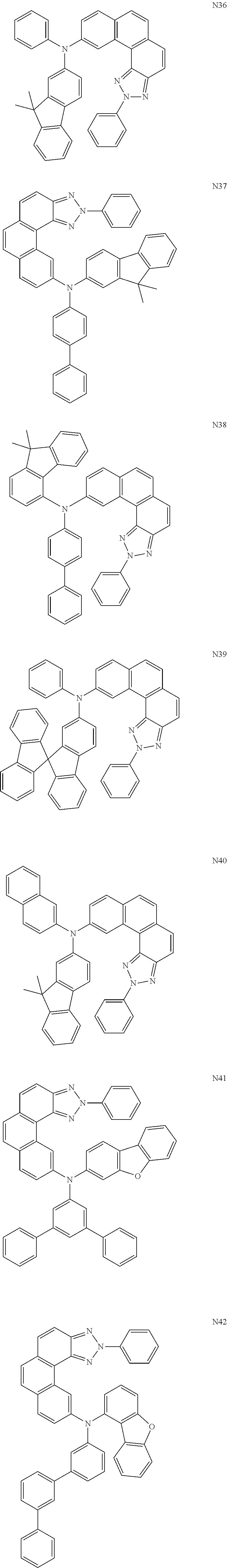 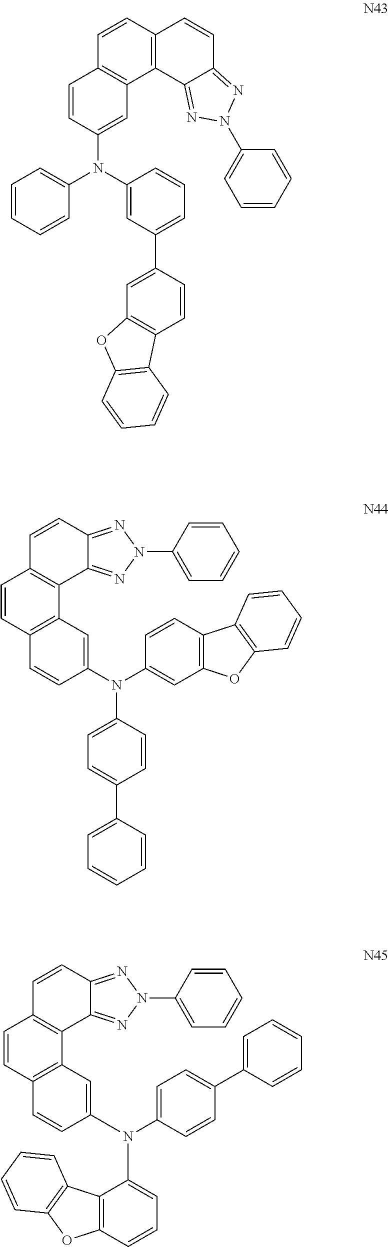 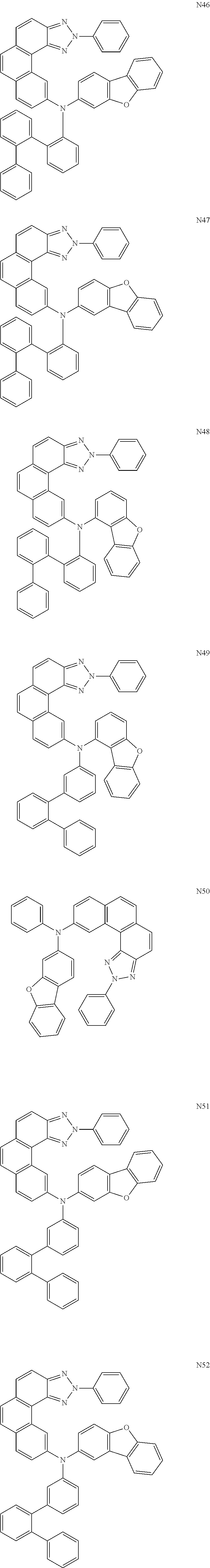 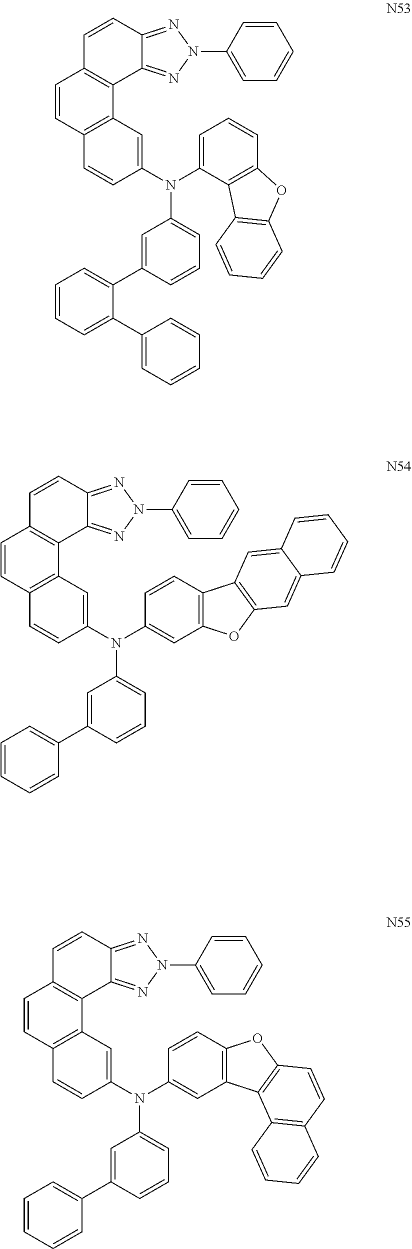 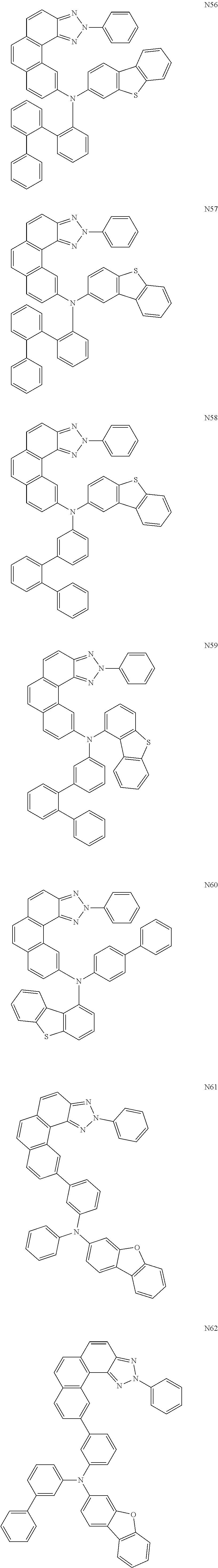 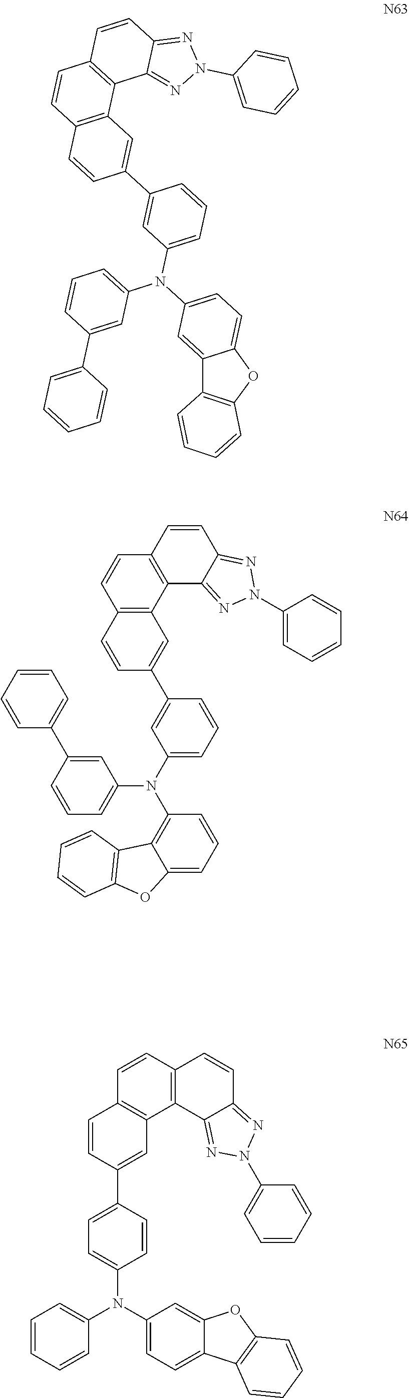 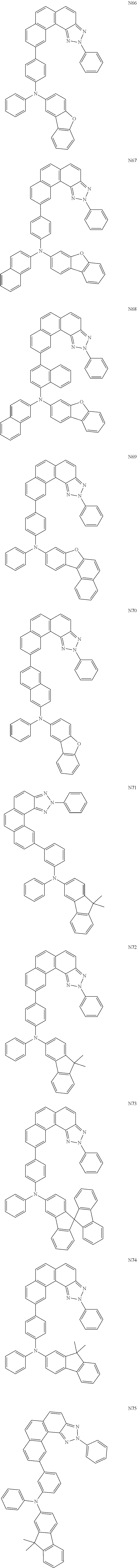 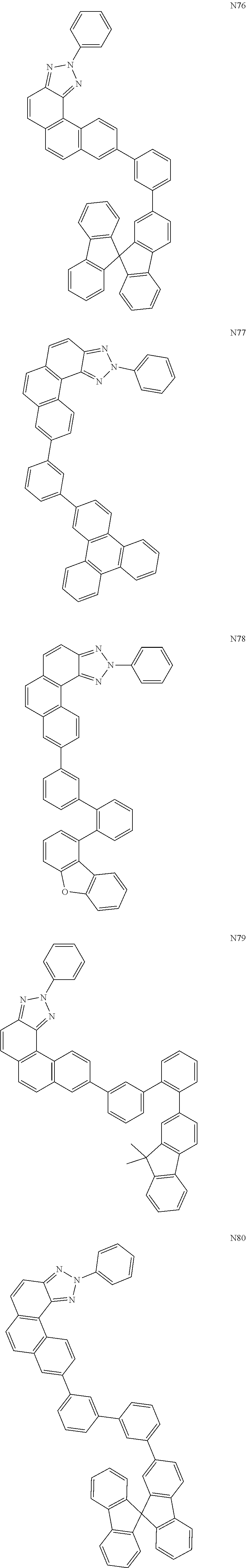 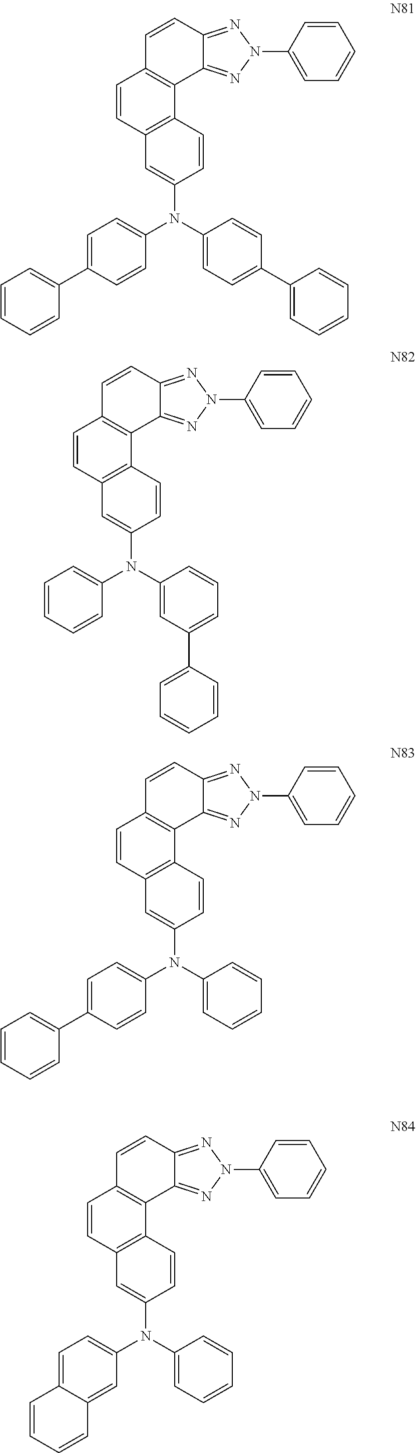 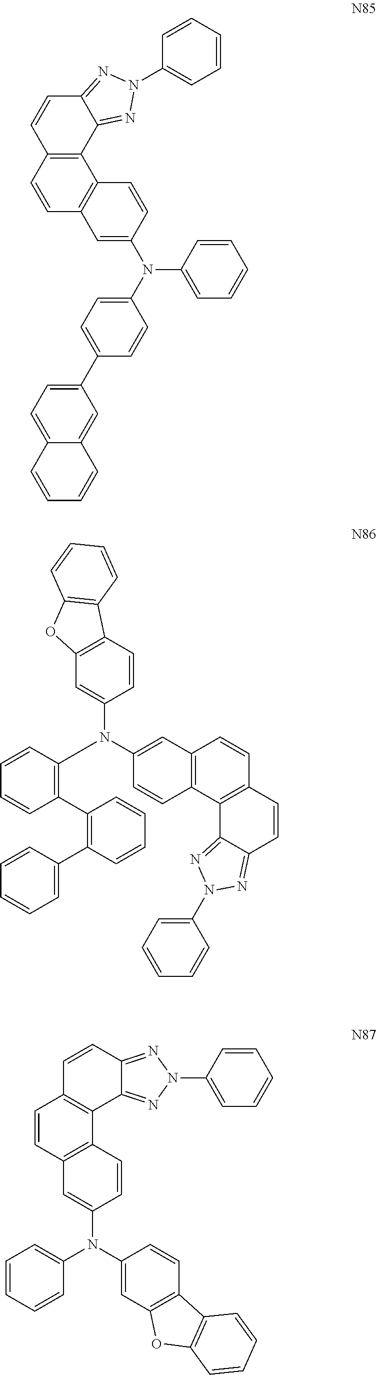 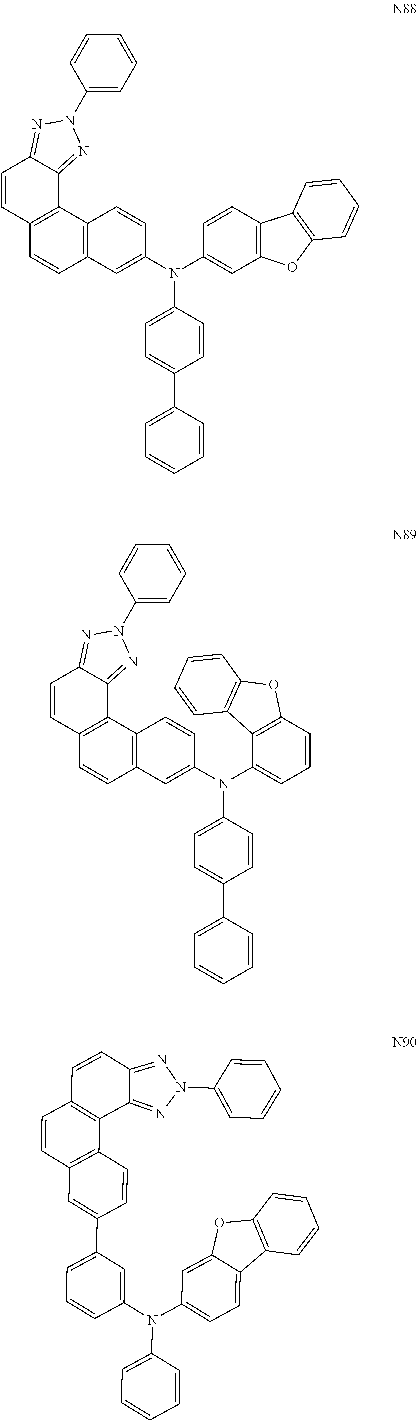 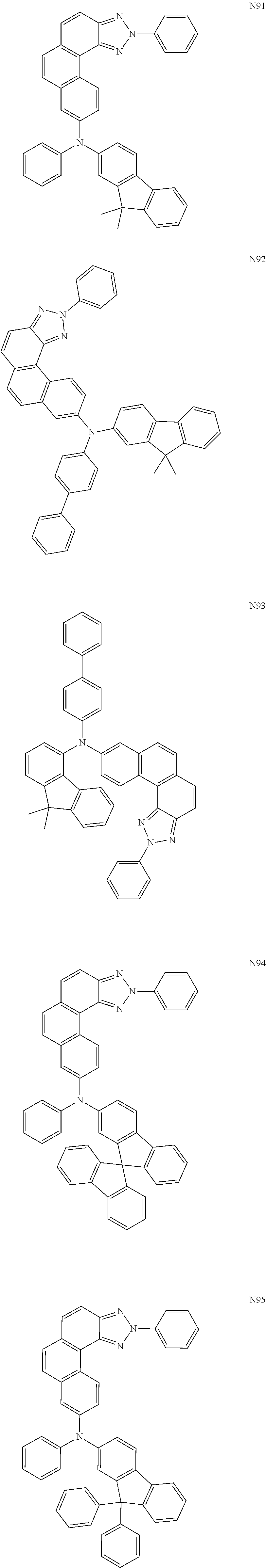 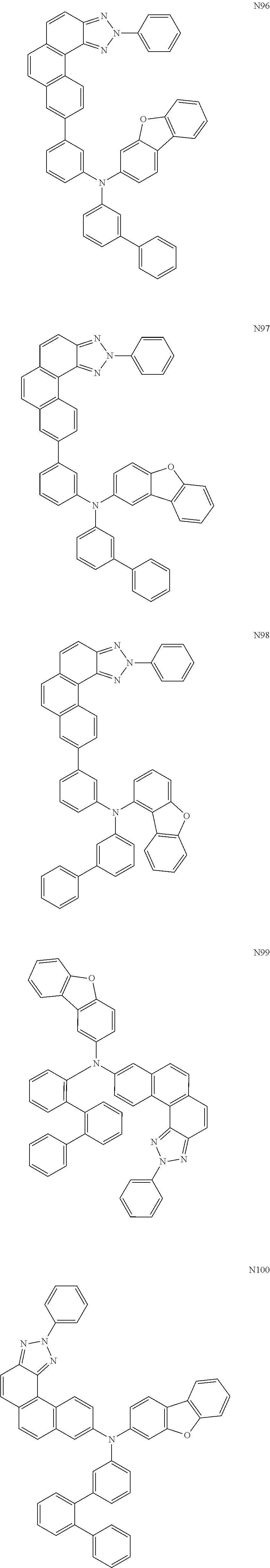 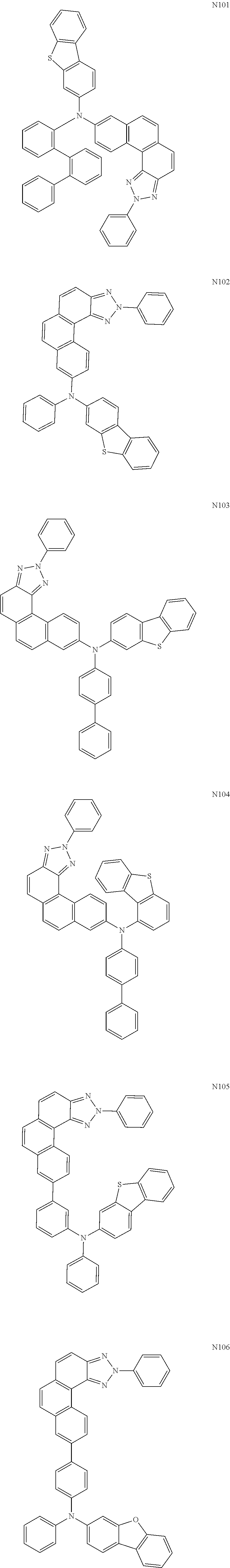 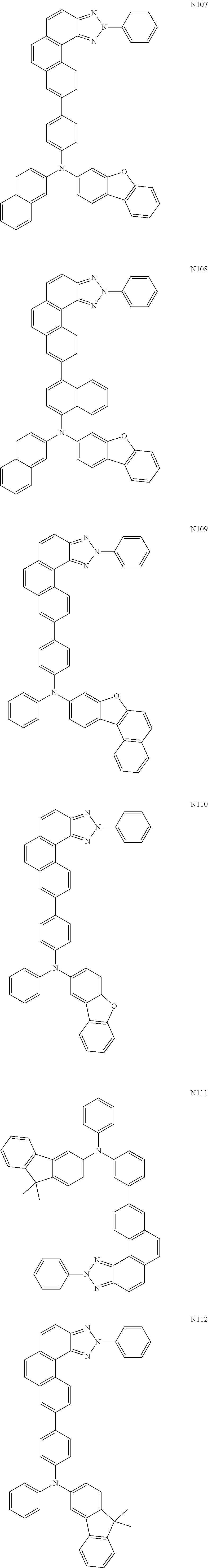 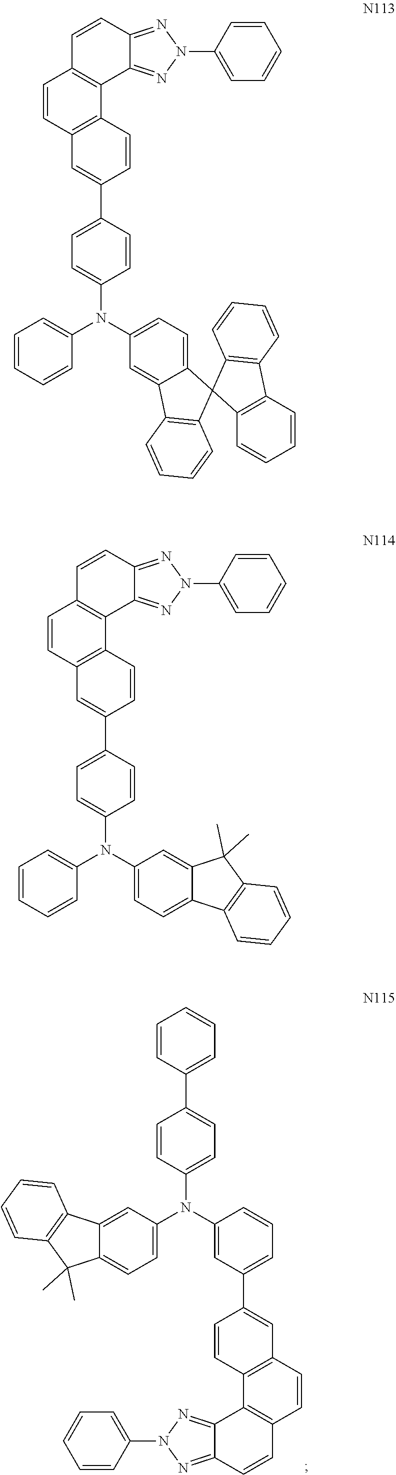
wherein D represents deuterium.

11. An organic electroluminescence material, characterized in that the organic electroluminescence material comprises the compound as claimed in claim 1.

12. The organic electroluminescence material as claimed in claim 11, characterized in that the organic electroluminescence material comprises at least one of compounds M1 to M206 and at least one of compounds N1 to N115.

13. An organic electroluminescence element, characterized in that the organic electroluminescence element comprises a first electrode, a second electrode and an organic layer disposed between the first electrode and the second electrode; and the organic layer is made of a material comprising the compound as claimed in claim 1.

14. An electronic device, characterized in that the electronic device comprises the organic electroluminescence element as claimed in claim 13.
